(19) United States
(12) Patent Application Publication
(10) Pub. No.: US 2026/0369406 A1
(43) Pub. Date: Nov. 19, 2026

(54) PAN-CANCER EARLY DETECTION AND MRD CFDNA METHYLATION

(71) Applicant: UNIVERSITY OF SOUTHERN CALIFORNIA, Los Angeles, CA (US)

(72) Inventors: Bodour SALHIA, Los Angeles, CA (US); David BUCKLEY, Los Angeles, CA (US)

(73) Assignee: UNIVERSITY OF SOUTHERN CALIFORNIA, Los Angeles, CA (US)

(21) Appl. No.: 19/158,740

(22) PCT Filed: Feb. 22, 2024
(86) PCT No.: PCT/US2024/016941

§ 371 Date: Aug. 21, 2025

Related U.S. Application Data
(60) Provisional application No. 63/486,379, filed on Feb. 22, 2023.

(51) Int. Cl. G16H 50/20; C12Q 1/6827; G06N 20/00; G16B 20/20; G16H 50/70

(52) U.S. Cl. CPC G16H 50/20; C12Q 1/6827; G06N 20/00; G16B 20/20; G16H 50/70

(57) ABSTRACT

Methods for determining whether a subject is likely to have or develop a pediatric cancer, adult cancer, or Minimal Residual Disease (MRD) using a trained a machine learning model configured to detect the said cancers.

### RELATED APPLICATIONS

This application claims priority under 35 U.S.C. § 119(e) to U.S. Provisional Patent Application No. 63/486,379, filed Feb. 22, 2023, which is incorporated herein by reference.

### BACKGROUND OF THE INVENTION

Cancer is the second leading cause of death in children aged 1-14 years in the United States, with approximately 11,000 new cases and 1,200 deaths annually. The 5-year overall survival rate for pediatric cancer has risen dramatically in recent years, from 58% in 1975 to just under 85% in 2020. However, neuroblastoma—the third most common form of pediatric cancer after leukemia and central nervous system (CNS) tumors—has a 5-year overall survival rate of 75% which plummets to 20% after the first disease recurrence. The improvement in overall survival for metastatic pediatric cancers has been mixed over the last few decades, with neuroblastoma showing improvement in prognosis due to treatment advances, while others like rhabdomyosarcoma and Ewing sarcoma have had minimal improvement. A key factor limiting progress in this is the rarity of many of these cancers, which limits research opportunities and hampers clinical trials.

Compared to adult-onset cancer, pediatric cancers typically have much lower mutational burden. While pediatric cancer genomes are characteristically ‘quiet’, with a low mutational burden, the epigenome of many pediatric cancers appears ‘loud’, with many driver mutations occurring in chromatin modifiers, such as SMARCB1 in malignant rhabdoid tumors, alongside widespread DNA methylation and histone marker changes. In light of this, targeting epigenetic modulators has been explored as a potential therapeutic option for multiple pediatric cancers.

DNA methylation changes are widespread and common in cancer and are among the earliest aberrations to occur in tumorigenesis. Typically, cancer methylomes display genome-wide hypomethylation and focal hypermethylation, particularly at promoter loci. The effectiveness of targeting DNA demethyltransferase inhibitors has been explored in multiple cancers—notably neuroblastoma, Ewing sarcoma, and AML.

Accordingly, there is a need for a new method of identifying pediatric cancers, adult cancers, recurrence of each that is more sensitive and has higher specificity than previous methods. The present disclosure satisfies these needs.

### SUMMARY OF THE INVENTION

The rarity of pediatric cancers poses a significant challenge to developing cancer-type specific diagnostic, prognostic or predictive biomarkers. In this context, it is appealing to consider a common molecular signature across multiple cancer types, an approach that has also shown promise in multi-cancer early detection testing. Specifically, we aimed to identify DNA methylation changes common to multiple pediatric cancers. Compared to adult-onset cancer, pediatric cancer genomes are characteristically ‘get’, with a low mutational burden, while the epigenome of many pediatric cancers appears ‘loud’, with many driver mutations occurring in chromatin modifiers, alongside widespread DNA methylation and histone marker changes. DNA methylation changes are widespread in cancer and are among the earliest aberrations to occur in tumorigenesis.

In the current disclosure, we aimed to identify DNA methylation changes common to multiple non-CNS pediatric solid cancers. We performed whole genome bisulfite sequencing (WGBS) of pediatric cancers, including 31 tumor tissue, 13 normal tissue, and 20 plasma cfDNA samples from 27 individuals, representing 11 different pediatric cancer subtypes. By integrating data across tumor types, we identified the minimal focal regions that were differentially methylated in the greatest number of samples across cancer types, which we termed minimally differentially methylated regions (mDMRs). We also found differential methylation of mDMRs in 518 pediatric cancer samples from 4 cancer types, sourced from ‘Therapeutically Applicable Research to Generate Effective Treatments’ (TARGET), and 6426 adult cancer samples, sourced from ‘The Cancer Genome Atlas’ (TCGA), from 14 cancer types. We performed further validation using a targeted hybridization probe capture assay in an independent set of 44 pediatric cancer tissue samples from 6 tumor types. Finally, we found that these methylation changes were detectable in cell-free (cf)DNA and could serve as potential cfDNA methylation biomarkers.

The identification of such a signature in pediatric cancers could potentially provide the basis for cfDNA methylation liquid biopsy for early detection, treatment response, or minimal residual disease monitoring. Thus, our study's endeavor to uncover such a methylation pattern has both scientific and clinical value, aiming to translate these molecular insights into tangible advances for pediatric cancer care.

Accordingly, in some embodiments, a method for determining whether a subject is likely to have or develop a pediatric cancer, adult cancer, or Minimal Residual Disease (MRD) comprises the steps of a) training a machine learning model to detect the pediatric cancer, the adult cancer, or the MRD, wherein the machine learning model is trained using target regions from a plurality of cancer samples and corresponding target regions from non-cancerous samples, wherein the cancer samples comprise at least two different cancer types, wherein the machine learning model is configured to identify the pediatric cancer, the adult cancer, or the MRD based on a comparison of a methylation pattern of target regions of the cancer samples compared to a methylation pattern of corresponding target regions of the non-cancerous samples; b) determining a methylation pattern of target regions of a deoxyribonucleic acid (DNA) sample obtained from the subject; c) applying the trained machine learning model to the methylation pattern of the target regions of the DNA obtained from the subject; and d) determining that the subject has or does not have the pediatric cancer, the adult cancer, or the MRD based on an output of the machine learning model.

In some embodiments, the methylation pattern of the plurality of each of target regions is determined using DNA methylation analysis, wherein the DNA methylation analysis comprises one or more of whole genome bisulfite sequencing (WGBS), Reduced Representation Bisulfite sequencing (RRBS), Targeted bisulfite sequencing, Hybridization Probe capture, Methylation bead arrays, and Enzymatic methyl-sequence conversion. In some embodiments, the methylation pattern of the plurality of target regions is determined using hybridization probe capture after whole genome bisulfite sequencing. In some embodiments, the hybridization probe capture comprises one or more probes that hybridize to the one or more target genomic regions, wherein each of the one or more probes comprises ribonucleic acid or deoxyribonucleic acid, and optionally, wherein each of the one or more probes comprises an affinity tag selected from the group consisting of biotin and streptavidin.

In some embodiments, the target regions of steps a-c comprise about 30% to about 50% of the target regions of Table 1, about 50% to about 70% of the target regions of Table 1, about 70% to about 90% of the target regions of Table 1, about 90% to about 95% of the target regions of Table 1, or the plurality of target regions comprises greater than about 95% of the target regions of Table 1.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-E. Analysis of differential DNA methylation patterns in pediatric cancers. (A) Hierarchical clustering of beta values across 183 highly variable DMRs in 31 tumor samples. Row annotation bar denotes diagnosis; column annotation bar denotes DMR cluster as determined by k-means clustering. (B) Uniform manifold approximation projection (UMAP) per sample based 183 most variable DMRs as in in C. (C) Heatmap of beta values of 166/183 regions in C (17 regions from C were dropped due to limitations of 450 k array) from 526 TARGET samples and 17 POETIC samples using. (D) UMAP of TARGET & POETIC samples across regions in E by diagnosis and (E) source.

FIG. 2A-C. WGBS analysis reveals pan pediatric cancer DNA methylation profile. (A) Number of overlapping mDMRs (y-axis) in a given number of samples (x-axis) by directionality (light blue=hypomethylated regions: red=hypermethylated regions). Red dotted line indicates cutoff selected (22 samples or greater: 70% of tumor samples) resulting in 402 hypomethylated and 503 hypermethylated regions. (B) Box plots of average beta values for hypo and hypermethylated mDMRs. Tumor/normal comparisons are statistically significant (p<0.0001; Wilcoxon rank sum test). (C) Receiver operating characteristic (ROC) curve of random forest classifier model constructed using mDMRs. ROC curve annotated with area under the curve (AUC). Significance annotation: ‘ns’:p>0.05, ‘*’:p≤0.05, ‘**’:p≤0.01, ‘***’:p≤0.001, ‘****’:p≤0.0001.

FIG. 3. mDMR differential methylation in TARGET and ENCODE datasets. Plots show mean beta values across hypermethylated (left) and hypomethylated (right) mDMRs in MRT, NBL OS, and WT tumor samples from the TARGET database. Normal tissues from POETIC and normal tissues from ENCODE were used as controls; all comparisons were statistically significant (Wilcox p-value≤0.0001) and consistent with the directionality observed in POETIC samples. Significance annotation: ‘ns’:p>0.05, ‘*’:p≤0.05, ‘**’:p≤0.01, ‘***’:p≤0.001, ‘****’:p≤0.0001.

FIG. 4A-C. mDMRs detected in multiple adult cancers from TCGA. (A) ROC curves from TCGA 450K DNA methylation data using a random forest model trained 422 of the 905 pediatric cancer mDMRs derived by WGBS (subset of 422 regions used due to the limitations of the 450K array). Plots annotated with TCGA cancer code and AUC. (B) Graphical representation of AUC in A with 95% CI indicated as error bars. See the GDC website for study abbreviation disambiguation (gdc.cancer.gov/resources-tcga-users/tcga-code-tables/tcga-study-abbreviations). (C) Tumor and normal mean beta values across hypermethylated (top) and hypomethylated (bottom) mDMRs in all 14 adult cancer types. All comparisons are statistically significant (Wilcox p-value≤0.0001), except PAAD hypomethylated comparison, and consistent with the directionality observed in POETIC samples. Significance annotation: ‘ns’:p>0.05, ‘*’:p≤0.05, ‘**’:p≤0.01, ‘***’:p≤0.001, ‘****’:p≤0.0001.

FIG. 5A-C. Overlapping DMRs between plasma and tissue. (A) Total DMRs called in cell free (cf)DNA. Stacked histograms displaying number of DMRs per sample or (B) percent of DMRs called in cfDNA that overlap with at least one DMR called in patient-matched tumor tissue. Light grey represents the hypermethylated regions and the dark grey represents the hypomethylated regions. (C) Correlation plots of differential methylation of intersecting DMRs between patient matched tissue and cfDNA. Each point represents Δβ in one intersecting region. Plots annotated with R², Spearman correlation, and line of best fit. Color denotes point density (yellow=high, purple=low).

FIG. 6A-B. Common features identified in solid tumors serve as biomarkers in plasma. (A/B) Mean beta from cfDNA across 402 hypomethylated and 905 hypermethylated mDMRs identified in tumor samples. All regions across all healthy and disease plasma samples are represented.

FIG. 7A-D. Targeted methylation analysis of hypomethylated mDMRs in an independent cohort. (A) Mean beta across 402 hypomethylated mDMRs in 44 additional tissue samples from 6 tumor types acquired from CHLA compared to nonnal tissue samples. All comparisons are statistically significant (Wilcox rank-sum test, Significance annotation: ‘ns’:p>0.05, ‘*’:p≤0.05, ‘**’:p≤0.01, ‘***’:p≤0.001. ‘****’:p≤0.0001). (B) Heatmap of beta values within mDMRs in POETIC and CHLA cohorts. Annotation bars show diagnosis, tumor/normal status, and source for each sample. UMAPs generated using mean beta values across all 402 colored by source (C) and cancer type (D).

FIG. 8A-B. Number of DMRs called in WGBS by sample and cancer type. (A) Number of DMRs per sample. Stacked histograms displaying number of DMRs per sample. Light grey represents the hypermethylated regions and the dark grey represents the hypomethylated regions. Chart is subdivided by tumor type: Embryonal rhabdomyosarcoma (ERMS), neuroblastoma (NBL) and osteosarcoma (OS), hepatoblastoma (HB), malignant rhabdoid tumor (MRT), and fibrolamellar hepatocellular carcinoma (FHC); all cancer types were grouped as ‘Other’. (B) Stacked Histogram displaying average number of DMRs per cancer type, where n denotes number of samples per tumor type.

FIG. 9A-B. MRT Methylation profiles. (A) Number of DMRs called in P01-019 (MRT). Shade delineates hypermethylated regions (light grey) from hypomethylated regions (dark grey). Each sample annotated with percent of DMR calls that are hypermethylated. (B) Genome-wide beta value of TARGET samples shows MRT hypermethylation.

FIG. 10. UMAP of DMRs identified in FIG. 1c by k-means cluster. Each point represents an individual DMR as detailed in FIG. 1c. Clusters on column annotation of FIG. 1c are ‘K-Means Cluster’.

FIG. 11A-B. mDMRs detected in multiple stage I adult cancers from TCGA. (A) ROC curves from TCGA 450K DNA methylation data using a random forest model trained 422 of the 905 pediatric cancer mDMRs derived by WGBS (subset of 422 regions used due to the limitations of the 450K array). Plots annotated with TCGA cancer code and AUC. (B) Graphical representation of AUC in A with 95% CI indicated as error bars. See the GDC website for study abbreviation disambiguation (gdc.cancer.gov/resources-tcga-users/tcga-code-tables/tcga-study-abbreviations).

FIG. 12A-B. mDMRs detected in multiple stage II adult cancers from TCGA. (A) ROC curves from TCGA 450K DNA methylation data using a random forest model trained 422 of the 905 pediatric cancer mDMRs derived by WGBS (subset of 422 regions used due to the limitations of the 450K array). Plots annotated with TCGA cancer code and AUC. (B) Graphical representation of AUC in A with 95% CI indicated as error bars. See the GDC website for study abbreviation disambiguation (gdc.cancer.gov/resources-tcga-users/tcga-code-tables/tcga-study-abbreviations).

FIG. 13A-B. mDMRs detected in multiple stage III adult cancers from TCGA. (A) ROC curves from TCGA 450K DNA methylation data using a random forest model trained 422 of the 905 pediatric cancer mDMRs derived by WGBS (subset of 422 regions used due to the limitations of the 450K array). Plots annotated with TCGA cancer code and AUC. (B) Graphical representation of AUC in A with 95% CI indicated as error bars. See the GDC website for study abbreviation disambiguation (gdc.cancer.gov/resources-tcga-users/tcga-code-tables/tcga-study-abbreviations).

FIG. 14A-B. mDMRs detected in multiple stage IV adult cancers from TCGA. (A) ROC curves from TCGA 450K DNA methylation data using a random forest model trained 422 of the 905 pediatric cancer mDMRs derived by WGBS (subset of 422 regions used due to the limitations of the 450K array). Plots annotated with TCGA cancer code and AUC. (B) Graphical representation of AUC in A with 95% CI indicated as error bars. See the GDC website for study abbreviation disambiguation (gdc.cancer.gov/resources-tcga-users/tcga-code-tables/tcga-study-abbreviations).

FIG. 15. curves from mDMRs in CNS tumor (Capper et al.) dataset. ROC curves from Capper et al. 450K DNA methylation data using a random forest model trained 422 of the 905 pediatric cancer mDMRs derived by WGBS (subset of 422 regions used due to the limitations of the 450K array). Each plot represents one methylation class from Capper et al.

FIG. 16. Cell free DNA yield from plasma. DNA yield in pg/ul per plasma sample. Vertically subdivided by diagnosis.

### DETAILED DESCRIPTION

Pediatric cancers typically have lower mutational burden compared to adult-onset cancers: however, the epigenomes in pediatric cancer are highly altered, with widespread DNA methylation changes. The rarity of pediatric cancers poses a significant challenge to developing cancer-type specific biomarkers for diagnosis, prognosis, or treatment monitoring. In the current study, we explored the potential of a common molecular signature across various pediatric cancers, particularly focusing on DNA methylation alterations. To do this, we conducted whole genome bisulfite sequencing (WGBS) on 31 pediatric tumor tissues, 13 normal tissues, and 20 plasma cell-free (cf)DNA samples, representing 11 different pediatric cancer types. We found the minimal focal regions that were differentially methylated across samples in multiple cancer types which we termed minimally differentially methylated regions (mDMRs). These methylation changes were also observed in 518 pediatric and 6426 adult cancer samples accessed from publicly available databases, and in 44 pediatric cancer samples we analyzed using a targeted hybridization probe capture assay. Finally, we found that these methylation changes were detectable in cfDNA and could serve as potential cfDNA methylation biomarkers.

### DEFINITIONS

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001 or Singleton, et al., Dictionary of Microbiology and Molecular Biology, 2d ed., John Wiley and Sons, New York (1994), and Hale & Markham, The Harper Collins Dictionary of Biology. Harper Perennial, N.Y. (1991). General laboratory techniques (DNA extraction, RNA extraction, cloning, cell culturing. etc.) are known in the art and described, for example, in Molecular Cloning: A Laboratory Manual, J. Sambrook et al., 4th edition, Cold Spring Harbor Laboratory Press, 2012.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”. “consisting essentially of” and “consisting of” may be replaced with either of the other two terms. The disclosure illustratively described herein may be suitably practiced in the absence of any element or elements, limitation, or limitations not specifically disclosed herein.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five substituents on the ring.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”. “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only: they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number 1” to “number 2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

As used herein, the term “a portion of” or “a portion thereof” means consecutive nucleotides of the sequence of said particular region. A portion according to the invention can comprise or consist of at least 15 or 20 consecutive nucleotides, preferably at least 100, 200, 300, 500 or 700 consecutive nucleotides, and more preferably at least 1, 2, 3, 4 or 5 consecutive kb of said particular region. For example, a portion can comprise or consist of 1, 2, 3, 4, 5, 6, 7, 8, 9, 10, 11, 12, 13, 14, 15 consecutive kb of said particular region.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis): (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping, or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “amplicon” refers to nucleic acid products resulting from the amplification of a target nucleic acid sequence. Amplification is often performed by PCR. Amplicons can range in size from 20 base pairs to 15000 base pairs in the case of long-range PCR but are more commonly 100-1000 base pairs for bisulfite-treated DNA used for methylation analysis.

The term “amplification” refers to an increase in the number of copies of a nucleic acid molecule. The resulting amplification products are called “amplicons.” Amplification of a nucleic acid molecule (such as a DNA or RNA molecule) refers to use of a technique that increases the number of copies of a nucleic acid molecule in a sample. An example of amplification is the polymerase chain reaction (PCR), in which a sample is contacted with a pair of oligonucleotide primers under conditions that allow for the hybridization of the primers to a nucleic acid template in the sample. The product of amplification can be characterized by such techniques as electrophoresis, restriction endonuclease cleavage patterns, oligonucleotide hybridization or ligation, and/or nucleic acid sequencing. In some embodiments, the methods provided herein can include a step of producing an amplified nucleic acid under isothermal or thermal variable conditions.

The term “biological sample” refers to a sample obtained from an individual. As used herein, biological samples include all clinical samples containing genomic DNA (such as cell-free genomic DNA) useful for cancer diagnosis and prognosis, including, but not limited to, cells, tissues, and bodily fluids, such as: blood, derivatives and fractions of blood (such as serum or plasma), buccal epithelium, saliva, urine, stools, bronchial aspirates, sputum, biopsy (such as tumor biopsy), and CVS samples. A “biological sample” obtained or derived from an individual includes any such sample that has been processed in any suitable manner (for example, processed to isolate genomic DNA for bisulfite treatment) after being obtained from the individual.

The term “bisulfite treatment” refers to the treatment of DNA with bisulfite or a salt thereof, such as sodium bisulfite (NaHSO₃). Bisulfite reacts readily with the 5,6-double bond of cytosine, but poorly with methylated cytosine. Cytosine reacts with the bisulfite ion to form a sulfonated cytosine reaction intermediate which is susceptible to deamination, giving rise to a sulfonated uracil. The sulfonate group can be removed under alkaline conditions, resulting in the formation of uracil. Uracil is recognized as a thymine by polymerases and amplification will result in an adenine-thymine base pair instead of a cytosine-guanine base pair.

The term “cancer” refers to a biological condition in which a malignant tumor or other neoplasm has undergone characteristic anaplasia with loss of differentiation, increased rate of growth, invasion of surrounding tissue, and which is capable of metastasis. A neoplasm is a new and abnormal growth, particularly a new growth of tissue or cells in which the growth is uncontrolled and progressive. A tumor is an example of a neoplasm. Non-limiting examples of types of cancer include lung cancer, stomach cancer, colon cancer, breast cancer, uterine cancer, bladder, head and neck, kidney, liver, ovarian, pancreas, prostate, and rectum cancer.

The terms “polynucleotide” and “nucleic acid” are used interchangeably and mean at least two or more ribo- or deoxy-ribo nucleic acid base pairs (nucleotide) linked which are through a phosphoester bond or equivalent. The nucleic acid includes polynucleotide and polynucleoside. The nucleic acid includes a single molecule, a double molecule, a triple molecule, a circular molecule, or a linear molecule. Examples of the nucleic acid include RNA, DNA. cDNA, a genomic nucleic acid, a naturally existing nucleic acid, and a non-natural nucleic acid such as a synthetic nucleic acid but are not limited. Short nucleic acids and polynucleotides (e.g., 10 to 20, 20 to 30, 30 to 50, 50 to 100 nucleotides) are commonly called “oligonucleotides” or “probes” of single-stranded or double-stranded DNA.

The term “DNA (deoxyribonucleic acid)” refers to a long chain polymer which comprises the genetic material of most living organisms. The repeating units in DNA polymers are four different nucleotides, each of which comprises one of the four bases, adenine, guanine, cytosine, and thymine bound to a deoxyribose sugar to which a phosphate group is attached. Triplets of nucleotides (referred to as codons) code for each amino acid in a polypeptide, or for a stop signal. The term codon is also used for the corresponding (and complementary) sequences of three nucleotides in the mRNA into which the DNA sequence is transcribed.

The term “cell-free DNA” refers to DNA which is no longer fully contained within an intact cell, for example DNA found in plasma or serum.

The ten “target nucleic acid molecule” refers to a nucleic acid molecule whose detection, amplification, quantitation, qualitative detection, or a combination thereof, is intended. The nucleic acid molecule need not be in a purified form. Various other nucleic acid molecules can also be present with the target nucleic acid molecule. For example, the target nucleic acid molecule can be a specific nucleic acid molecule of which the amplification and/or evaluation of methylation status is intended. Purification or isolation of the target nucleic acid molecule, if needed, can be conducted by methods known to those in the art, such as by using a commercially available purification kit or the like.

The term “methylation level” refers to the state of methylation (methylated or not methylated) of the cytosine nucleotide of one or more CpG sites within a genomic sequence.

The term “hypomethylated” or “hypermethylated” refers to a methylation status of a DNA molecule containing multiple CpG sites (e.g., more than 3, 4, 5, 6, 7, 8, 9, 10, etc.) where a high percentage of the CpG sites (e.g., more than 80%, 85%, 90%, or 95%, or any other percentage within the range of 50%-100%) are unmethylated or methylated, respectively.

The term “CpG Site” refers to a di-nucleotide DNA sequence comprising a cytosine followed by a guanine in the 5′ to 3′ direction. The cytosine nucleotides of CpG sites in genomic DNA are the target of intracellular methyltransferases and can have a methylation status of methylated or not methylated. Reference to “methylated CpG site” or similar language refers to a CpG site in genomic DNA having a 5-methylcytosine nucleotide.

As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated. e.g., as implemented in the program PC/GENE (Intelligenetics, Mountain View, Calif).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” in the context of a peptide indicates that a peptide comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even 95%, 96%, 97%, 98% or 99%, sequence identity to the reference sequence over a specified comparison window. In certain embodiments, optimal alignment is conducted using the homology alignment algorithm of Needleman and Wunsch (Needleman and Wunsch, J M B, 48, 443 (1970)). An indication that two peptide sequences are substantially identical is that one peptide is immunologically reactive with antibodies raised against the second peptide. Thus, a peptide is substantially identical to a second peptide, for example, where the two peptides differ only by a conservative substitution. Thus, embodiment of the invention also provides nucleic acid molecules and peptides that are substantially identical to the nucleic acid molecules and peptides presented herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

The term “multiplex” refers to the use of more than one pair of primers intended to amplify multiple target gene segments simultaneously within a single tube. In this manner, all the primers may be contained within one tube to which a sample is introduced or positioned. All desired influenza virus and control gene segments are then amplified via the plurality of forward and reverse primers within the tube.

The term “complement” as used herein means the complementary sequence to a nucleic acid according to standard Watson/Crick base pairing rules. A complement sequence can also be a sequence of RNA complementary to the DNA sequence or its complement sequence and can also be a cDNA. The term “,substantially complementary” as used herein means that two sequences hybridize under stringent hybridization conditions. The skilled artisan will understand that substantially complementary sequences need not hybridize along their entire length. In particular, substantially complementary sequences comprise a contiguous sequence of bases that do not hybridize to a target or marker sequence, positioned 3′ or 5′ to a contiguous sequence of bases that hybridize under stringent hybridization conditions to a target or marker sequence.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include incubation temperatures of about 25 °C to about 37 °C; hybridization buffer concentrations of about 6×SSC to about 10×SSC; formanide concentrations of about 0% to about 25%; and wash solutions from about 4×SSC to about 8×SSC. Examples of moderate hybridization conditions include incubation temperatures of about 40 °C to about 50 °C; buffer concentrations of about 9×SSC to about 2×SSC; formamide concentrations of about 30% to about 50%; and wash solutions of about 5×SSC to about 2×SSC. Examples of high stringency conditions include incubation temperatures of about 55 °C to about 68 °C; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

As used herein, the term “reference genome” refers to any particular known, sequenced or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. One exemplary human reference genome is GRCh37 (UCSC equivalent: hg19).

As used herein, the term “normal reference standard” intends a control level, degree, or range of DNA methylation at a particular genomic region or gene in a sample that is not associated with cancer. The term “normal reference cutoff value” refers to a control threshold level of DNA methylation at a particular genomic region or gene or a differential methylation value (DMV). In some embodiments, DNA methylation levels enriched above the normal reference cutoff value are associated with having or developing cancer. In some embodiments, DNA methylation levels at or below the normal reference cutoff value are associated with not having or developing cancer.

“Detecting” as used herein refers to determining the presence and/or degree of methylation in a nucleic acid of interest in a sample. Detection does not require the method to provide 100% sensitivity and/or 100% specificity.

“RT-PCR” refers to reverse transcription polymerase chain reaction and is used to detect specific RNA, in this case specific gene segments of the influenza virus genome, such as by reverse transcribing the RNA of interest into its DNA complement through the use of reverse transcriptase. The newly synthesized cDNA can be amplified using traditional PCR. In an aspect, the RT-PCR provided herein is by a one-step approach, wherein the entire reaction from cDNA synthesis to PCR amplification occurs in a single tube. Alternatively, the process described herein is compatible with a two-step reaction requires that the reverse transcriptase reaction and PCR amplification be performed in separate tubes. Real-Time PCR: Current Technology and Applications, Logan, Edwards, and Saunders eds., Caister Academic Press, 2009; Bustin A-Z of Quantitative PCR (IUL Biotechnology, No. 5).

As used here, a “fragment” of DNA refers to a piece of cell-free DNA that is about 10 bp, about 20 bp, about 30 bp, about 40 bp, about 50 bp, about 60 bp, about 70 bp, about 80 bp, about 90 bp, about 100 bp, about 110 bp, about 120 bp, about 130 bp, about 140 bp, about 150 bp, about 160 bp, about 170 bp, about 180 bp, about 190 bp, about 200 bp, about 210 bp, about 220 bp, about 230 bp, 240 bp, about 250 bp, about 260 bp, about 270 bp, 280 bp, about 290 bp, about 300 bp, about 310 bp, about 320 bp, about 330 bp, about 340 bp, about 350 bp, about 360 bp, about 370 bp, about 380 bp, about 390 bp, or about 400 bp in length. Typically, DNA fragments are about 100 bp to about 200 bp, about 120 bp to about 180 bp, or about 140 bp to about 160 bp.

The term “neoadjuvant treatment” refers to treatment (such as chemotherapy or hormone therapy) administered before primary cancer treatment (such as surgery) to enhance the outcome of primary treatment.

The term “chemotherapy” refers to the treatment of cancer with an antitumor or chemotherapeutic agent as part of a standardized regimen. Chemotherapy may be given with a curative intent or it may aim to prolong life or to palliate symptoms. It may be used in conjunction with other cancer treatments, such as radiation therapy or surgery.

The term “methylation” refers to the addition of a methyl group to the 5′ carbon of the cytosine base in a deoxyribonucleic acid sequence of CpG within a genome.

The term “neighboring CpG site” refers to the collection of CpG sites within a genomic feature or over a short genetic distance. The genomic feature may be a promoter, an enhancer, an exon, an intron, a 5′-untranslated region (UTR), a 3′-UTR, a gene body, a stem cell associated region, a CpG island, a CpG shelf, a CpG shore, a LINE, a SINE, or an LTR. The short genetic distance may be 10 bp, 11 bp, 12 bp, 13 bp, 14 bp, 15 bp, 16 bp, 17 bp, 18 bp, 19 bp, 20 bp, 21 bp, 22 bp, 23 bp, 24 bp, 25 bp, 26 bp, 27 bp, 28 bp, 29 bp, 30 bp, 31 bp, 32 bp, 33 bp, 34 bp, 35 bp, 36 bp, 37 bp, 38 bp, 39 bp, 40 bp, 41 bp, 42 bp, 43 bp, 44 bp, 45 bp, 46 bp, 47 bp, 48 bp, 49 bp, 50 bp, 51 bp, 52 bp, 53 bp, 54 bp, 55 bp, 56 bp, 57 bp, 58 bp, 59 bp, 60 bp, 61 bp, 62 bp, 63 bp, 64 bp, 65 bp, 66 bp, 67 bp, 68 bp, 69 bp, 70 bp, 71 bp, 72 bp, 73 bp, 74 bp, 75 bp, 76 bp, 77 bp, 78 bp, 79 bp, 80 bp, 81 bp, 82 bp, 83 bp, 84 bp, 85 bp, 86 bp, 87 bp, 88 bp, 89 bp, 90 bp, 91 bp, 92 bp, 93 bp, 94 bp, 95 bp, 96 bp, 97 bp, 98 bp, 99 bp, 100 bp, 250 bp, 500 bp, 750 bp or 1,000 bp. Optionally, neighboring CpG sites occur within a sequencing read.

The term “Minimal Residual Disease” or “MRD” refers to cancer cells (e.g., breast cancer cells) remaining after treatment that cannot be detected using the scans or tests to identify a remission state (i.e., cancer free). Treatment of any cancer listed herein may result in MRD.

### Embodiments of the Invention

The disclosure provides for assays and various methods for detecting differences in methylation patterns of a target region of DNA. The differences in methylation patterns of the target regions of the sample (e.g. cfDNA, genomic DNA isolated from tissue) can indicate, for example, the presence or absence of a pediatric cancer, the presence or absence of an adult cancer, and/or a recurrence of a pediatric cancer or an adult cancer, such as minimal residual disease. The methylation pattern of the target region of DNA in a sample may be analyzed using a trained machine learning model that is trained using a defined set target regions of DNA of cancerous and non-cancerous control samples.

Generally, a test sample from a subject may be processed to determine the presence or absence of a pediatric cancer, an adult cancer, or MRD according to the following steps i) extracting nucleic acids (e.g., DNA) from a test sample from a subject suspected of having a pediatric cancer, adult cancer, or MRD; ii) converting unmethylated cytosines of the extracted nucleic acids to uracil (e.g., via bisulfite conversion); iii) preparing a library of bisulfite converted DNA: iv) enriching target nucleic acids by hybridizing the bisulfite converted DNA with hybridization probes: v) generating sequence reads of the enriched nucleic acids comprising the target regions: vi) aligning the sequence reads of the target regions with corresponding target regions of a reference genome (e.g., using Bismark); vii) perform differentially methylated region analysis (e.g., using Metilene) of the test sample compared to a normal sample or pool of normal samples to identify differentially methylated regions between the test (cancer) sample and the nonnal samples; vi) define the minimally differentially methylated regions (mDMRs) across samples; vii) building a classifier model with mDMRs using machine learning tools wherein an AUC value of 0.8 or greater indicates the presence of the pediatric cancer, the adult cancer, or MRD.

In one embodiment, a method for determining whether a subject is likely to have or develop a pediatric cancer, an adult cancer, and/or Minimal Residual Disease (MRD) comprises the steps of training a machine learning model to detect the pediatric cancer, the adult cancer, or the MRD, wherein the machine learning model is trained using target regions from a plurality of cancerous samples and corresponding target regions from non-cancerous samples, wherein the cancer samples comprise at least two different cancer types, wherein the machine learning model is configured to identify the pediatric cancer, the adult cancer, or the MRD based on a comparison of a methylation pattern of target regions of the cancerous samples compared to a methylation pattern of corresponding target regions of the non-cancerous samples; determining a methylation pattern of target regions of a deoxyribonucleic acid (DNA) sample obtained from the subject; applying the trained machine learning model to the methylation pattern of the target regions of the DNA obtained from the subject; and determining that the subject has or does not have the pediatric cancer, the adult cancer, or the MRD based on the output of the machine learning model.

Embodiments of the disclosure may comprise the steps of bisulfite conversion of the nucleic acids from a DNA sample of a subject using, for example, Whole Genome Bisulfite Sequencing (WGBS) or hybrid probe capture; next generation sequencing the converted and/or enriched nucleic acids; collecting the methylation data from the targeted regions (e.g., the target regions listed in Table 1); and using a trained machine learning model to determine, for example, the presence or absence of a pediatric cancer, a recurrence of a pediatric cancer, and/or the presence of an adult cancer or recurrence of an adult cancer.

In some embodiments, the method used to determine the methylation pattern of the one or more target nucleic acids includes methylation sequencing. For example, the methylation pattern of CpG sites within the target regions listed in Table 1 may be detected using DNA methylation sequencing. DNA methylation sequencing can involve, for example, treating DNA from a sample with bisulfite to convert unmethylated cytosine to uracil followed by amplification (such as PCR amplification) of a target nucleic acid within the treated genomic DNA, and sequencing of the resulting amplicon. Sequencing produces nucleotide reads that may be aligned to a genomic reference sequence that may be used to quantitate methylation levels of all the CpGs within an amplicon. Cytosines in non-CpG context may be used to track bisulfite conversion efficiency for each individual sample. The procedure is both time and cost-effective, as multiple samples may be sequenced in parallel using a 96 well plate and generates reproducible measurements of methylation when assayed in independent experiments.

Nucleic acid molecules may be subjected to conditions sufficient to convert unmethylated cytosines in the nucleic acid molecules to uracils (e.g., subsequent to extraction from a sample). For example, to detect DNA methylation, certain embodiments provide for first converting the DNA to be analyzed so that the unmethylated cytosine is converted to uracil. In one embodiment, a chemical reagent that selectively modifies either the methylated or non-methylated form of CpG dinucleotide motifs may be used. Suitable chemical reagents include hydrazine and bisulphite ions and the like. Preferably, isolated DNA is treated with sodium bisulfite (NaHSO₃) which converts unmethylated cytosine to uracil, while methylated cytosines are maintained. Without wishing to be bound by a theory, it is understood that sodium bisulfite reacts readily with the 5,6-double bond of cytosine, but poorly with methylated cytosine. Cytosine reacts with the bisulfite ion to form a sulfonated cytosine reaction intermediate that is susceptible to deamination, giving rise to a sulfonated uracil. The sulfonated group can be removed under alkaline conditions, resulting in the formation of uracil. The nucleotide conversion results in a change in the sequence of the original DNA. It is general knowledge that the resulting uracil has the base pairing behavior of thymine, which differs from cytosine base pairing behavior. To that end, uracil is recognized as a thymine by DNA polymerase. Therefore, after PCR or sequencing, the resultant product contains cytosine only at the position where 5-methylcytosine occurs in the starting template DNA. This makes the discrimination between unmethylated and methylated cytosine possible.

Nucleic acid molecules may also be subjected to further processing including other derivatization processes (e.g., to incorporate, modify, and/or delete one or more sequences, tags, or labels). In some cases, functional sequences (e.g., sequencing adapters, flow cell adapters, sequencing primers, etc.) may be added to nucleic acid molecules to facilitate nucleic acid sequencing. Accordingly, derivatives of nucleic acid molecules from a sample may comprise processed nucleic acid molecules including bisulfite-modified nucleic acid molecules, reverse-transcribed nucleic acid molecules, tagged nucleic acid molecules, barcoded nucleic acid molecules, and other modified nucleic acid molecules.

In some embodiments, methylation pattern of a target region may be determined using one or more of hybrid probe capture, targeted bisulfite amplicon sequencing, bisulfite DNA treatment, WGBS, bisulfite conversion combined with bisulfite restriction analysis (COBRA), bisulfite PCR, bisulfite modification, bisulfite pyrosequencing, methylated CpG island amplification, CpG binding column based isolation of CpG islands, CpG island arrays with differential methylation hybridization, high performance liquid chromatography, DNA methyltransferase assay, methylation sensitive PCR, cloning differentially methylated sequences, methylation detection following restriction, restriction landmark genomic scanning, methylation sensitive restriction fingerprinting, or Southern blot analysis.

In one embodiment, the method used to determine the methylation level of the one or more target regions in the DNA is WGBS (Cokus, et al. 2008. Nature 452(7184): 215-219; Lister, et al. 2009. Nature 462(7271): 315-322: Harris, et al. 2010. Nat Biotechnol 28(10): 1097-1105).

Other methods to assay the methylation status of CpG sites can also be used. Numerous DNA methylation detection methods are known in the art, including but not limited to hybrid probe capture (REF), methylation-specific enzyme digestion (Singer-Sam et al., Nucleic Acids Res. 18(3): 687, 1990; Taylor et al., Leukemia 15(4): 583-9, 2001), methylation-specific PCR (MSP or MSPCR) (Herman et al., Proc Natl Acad Sci USA 93(18): 9821-6, 1996), methylation-sensitive single nucleotide primer extension (MS-SnuPE) (Gonzalgo et al., Nucleic Acids Res. 25(12): 2529-31, 1997), restriction landmark genomic scanning (RLGS) (Kawai, Mol Cell Biol. 14(11): 7421-7, 1994; Akama, et al., Cancer Res. 57(15): 3294-9, 1997), and differential methylation hybridization (DMH) (Huang et al., Hum Mol Genet. 8(3): 459-70, 1999). In some embodiments, the methylation levels may be determined using one or more DNA methylation sequencing assays with or without bisulfite treatment of DNA.

In one embodiment, Reduced Representation Bisulfite Sequencing (RRBS) is used to measure methylation levels of a target region. Generally, RRBS begins with the treatment of nucleic acid with bisulfite to convert all unmethylated cytosines into uracil, followed by restriction enzyme digestion (for example, by an enzyme that recognizes a site that includes a CG sequence such as MspI) and complete fragment sequencing after coupling with an adapter ligand. The selection of the restriction enzyme enriches the fragments of the dense regions in CpG, reducing the number of redundant sequences that can map multiple positions of the gene during the analysis. Therefore, RRBS reduces the sample complexity of the nucleic acid sample by selecting a subset (e.g., by size selection using preparative gel electrophoresis) of restriction fragments for sequencing. In opposition to the sequencing of the complete genome with bisulfite, each fragment produced by restriction enzyme digestion contains information on DNA methylation for at least one CpG dinucleotide. Therefore, RRBS enriches the sample in promoters. CpG islands, and other genomic characteristics with a high frequency of restriction enzyme cleavage sites in these regions and, thus, provides an assay to assess the methylation status of one or more genomic loci.

A typical protocol for RRBS comprises the steps of digesting a sample of nucleic acid with a restriction enzyme such as Mspl, filling with projections and A-tails, ligating adapters, conversion with bisulfite, and PCR. See, for example. Gu et al. (2010), Nat Methods 7: 133-6; Meissner et al (2005), Nucleic Acids Res. 33: 5868-77.

In some embodiments, identifying the presence of a pediatric cancer, an adult cancer, or MRD in a subject may comprise using hybrid capture probes configured to selectively enrich nucleic acid molecules (e.g., DNA or RNA molecules) or sequences thereof. Such probes may be pull-down probes (e.g., bait sets). Selectively enriched nucleic acid molecules or sequences thereof may correspond to one or more target regions in the methylation profile of the data set. The presence of particular sequences, modifications (e.g., methylation states), deletions, additions, single nucleotide polymorphisms, copy number variations, or other features in the selectively enriched nucleic acid molecules or sequences thereof may be indicative of a presence and/or recurrence of a pediatric cancer. The probes may be selective for a subset of certain target regions of Table 1 in the DNA sample and/or for differentially methylated regions (e.g., CpG sites, CpA, sites, CpT sites, and/or CpC sites). The probes may be configured to selectively enrich nucleic acid molecules (e.g., DNA or RNA molecules) or sequences thereof corresponding to a plurality of target nucleic acid of target genomic sequences, such as the subset of the one or more genomic regions in the cell-free biological sample and/or differentially methylated regions (e.g., CpG sites, CpA, sites, CpT sites, and/or CpC sites). The probes may be nucleic acid molecules (e.g., DNA or RNA molecules) having sequence complementarity with target nucleic acid sequences. These nucleic acid molecules may be primers or enrichment sequences. The assaying of the nucleic acid molecules of the sample (e.g., cell-free biological sample) using probes that are selected for target nucleic acid sequences may comprise use of array hybridization, polymerase chain reaction (PCR), or nucleic acid sequencing (e.g., DNA sequencing or RNA sequencing). The number of target nucleic acid sequences selectively enriched using such a scheme may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 50, at least 100, at least 150, at least 200, at least 300, at least 500, or more than 500 different target nucleic acid sequences of the target genomic regions. Use of such probes for enrichment of target nucleic acids may be termed “hybrid capture”. Use of such hybrid capture probes may take place prior to or after bisulfite conversion (if applicable). Examples of target nucleic acid sequences include those associated with the target regions included in Table 1. In some embodiments, the target region includes all the sequences of Table 1. In some embodiments, the hybridization probes comprise an assay panel and are complementary to one or more target regions of Table 1, and may comprise at least 50, 60, 70, 80, 90, 100, 120, 150, or 200 pairs of probes. In some embodiments, the hybridization probes comprise an assay panel and may comprise at least 1,000, 2,000, 2,500, 5,000, 6,000, 7,500, 10,000, 15,000, 20,000, 25,000 or 50,000 different pairs of probes. In some embodiments, an assay panel may include at least 100, 120, 140, 160, 180, 200, 240, 300, or 400 different probes. In other embodiments, an assay panel may include at least 1,000, 2,000, 5,000, 10,000, 12,000, 15,000, 20,000, 30,000, 40,000, 50,000, or 100,000 different probes. Preferably, the number of probes is sufficient to overlap substantially all of the target regions of interest.

In some embodiments, the one or more probes comprises deoxyribonucleic acid and/or ribonucleic acid. In some embodiments, each of the one or more probes comprises an affinity tag selected from the group consisting of biotin and streptavidin.

Thus, in some embodiments, the methylation sequencing of the plurality of target regions uses one or more of whole genome sequencing, wherein the whole genome sequencing comprises one or more of whole genome bisulfite sequencing (WGBS), Reduced Representation Bisulfite sequencing (RRBS), Targeted bisulfite sequencing, Hybridization Probe capture, Methylation bead arrays, and Enzymatic methyl-sequence conversion.

Nucleic acid molecules (e.g., extracted cfDNA) or derivatives thereof may be subjected to sequencing to provide a plurality of sequencing reads. Sequencing reads may be aligned with and/or analyzed with regard to a reference genome. Based at least in part on sequencing reads, an absolute amount or relative amount of nucleic acid molecules (including an absolute or relative level of methylation within said molecules) corresponding to one or more genomic regions may be measured. Alternatively, sequencing reads may not be used to determine an amount or relative amount of nucleic acid molecules. A data set comprising a genomic profile (e.g., methylation profile) of one or more genomic regions of a sample may be generated based at least in part on sequencing reads. Sequencing reads may be processed to identify methylation patterns of the target regions of the DNA in a sample.

Sequence identification may be performed by sequencing, array hybridization (e.g., Affymetrix), or nucleic acid amplification (e.g., PCR), for example. Sequencing may be performed by any suitable sequencing methods, such as massively parallel sequencing (MPS), paired-end sequencing, high-throughput sequencing, next-generation sequencing (NGS), shotgun sequencing, single-molecule sequencing, nanopore sequencing, nanopore sequencing with direct detection or inference of methylation status, semiconductor sequencing, pyrosequencing, sequencing-by-synthesis (SBS), sequencing-by-ligation, sequencing-by hybridization, and RNA-Seq (Illumina).

Sequencing and/or preparing a nucleic acid sample for sequencing may comprise performing one or more nucleic acid reactions such as one or more nucleic acid amplification processes (e.g., of DNA or RNA molecules). Nucleic acid amplification may comprise, for example, reverse transcription, primer extension, asymmetric amplification, rolling circle amplification, ligase chain reaction, polymerase chain reaction (PCR), and multiple displacement amplification. Examples of PCR methods include digital PCR (dPCR), emulsion PCR (ePCR), quantitative PCR (qPCR), real-time PCR (RT-PCR), hot start PCR, multiplex PCR, asymmetric PCR, nested PCR, and assembly PCR. A suitable number of rounds of nucleic acid amplification (e.g., PCR, such as qPCR, RT-PCR, dPCR, etc.) may be performed to sufficiently amplify an initial amount of nucleic acid molecule (e.g., DNA molecule) or derivative thereof to a desired input quantity for subsequent sequencing. In some cases, the PCR may be used for global amplification of nucleic acid molecules. This may comprise using adapter sequences that may be first ligated to different molecules followed by PCR amplification using universal primers. PCR may be performed using any of a number of commercial kits, e.g., provided by Life Technologies, Affymetrix, Promega, Qiagen, etc. In other cases, only certain target nucleic acids within a population of nucleic acids may be amplified. Specific primers, possibly in conjunction with adapter ligation, may be used to selectively amplify certain targets for downstream sequencing. In some cases, nested primers may be used to target specific genomic regions. Nucleic acid amplification may comprise targeted amplification of one or more genetic loci, genomic regions, cfDNA target regions, or differentially methylated regions (e.g., CpG sites, CpA, sites, CpT sites, and/or CpC sites), and in particular, the target regions listed in Table 1. In some cases, nucleic acid amplification is performed after bisulfite conversion. Such a procedure may be termed targeted bisulfite amplicon sequencing (TBAS). Nucleic acid amplification may comprise the use of one or more primers, probes, enzymes (e.g., polymerases), buffers, and deoxyribonucleotides. Nucleic acid amplification may be isothermal or may comprise thermal cycling. Thermal cycling may involve changing a temperature associated with various processes of nucleic acid amplification including, for example, initialization, denaturation, annealing, and extension. Sequencing may comprise use of simultaneous reverse transcription (RT) and PCR, such as a OneStep RT-PCR kit protocol by Qiagen, NEB, Thermo Fisher Scientific, or Bio-Rad.

Nucleic acid molecules (e.g., DNA or RNA molecules) or derivatives thereof may be labeled or tagged, e.g., with identifiable tags, to allow for multiplexing of a plurality of samples. For example, every nucleic acid molecule or derivative thereof associated with a given sample or subject may be tagged or labeled (e.g., with a barcode such as a nucleic acid barcode sequence or a fluorescent label). Nucleic acid molecules or derivatives thereof associated with other samples or subjects may be tagged or labels with different tags or labels such that nucleic acid molecules or derivatives thereof may be associated with the sample or subject from which they derive. Such tagging or labeling also facilitates multiplexing such that nucleic acid molecules or derivatives thereof from multiple samples and/or subjects may be analyzed (e.g., sequenced) at the same time. Any number of samples may be multiplexed. For example, a multiplexed reaction may contain nucleic acid molecules or derivatives thereof from at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 initial samples. Such samples may be derived from the same or different subjects. For example, a plurality of samples may be tagged with sample barcodes (e.g., nucleic acid barcode sequences) such that each nucleic acid molecule (e.g., DNA molecule) or derivative thereof may be traced back to the sample (and/or the subject) from which the nucleic acid molecule originated. Sample barcodes may permit samples from multiple subjects to be differentiated from one another, which may permit sequences in such samples to be identified simultaneously, such as in a pool. Tags, labels, and/or barcodes may be attached to nucleic acid molecules or derivatives thereof by ligation, primer extension, nucleic acid amplification, or another process. In some cases, nucleic acid molecules or derivatives thereof of a particular sample may be tagged, labeled, or barcoded with different tags, labels, or barcodes (e.g., unique molecular identifiers) such that different nucleic acid molecules or derivatives thereof deriving from the same sample may be differentially tagged, labeled, or barcoded. In some cases, nucleic acid molecules or derivatives thereof from a given sample may be labeled with both different labels and identical labels, such that each nucleic acid molecule or derivative thereof associated with the sample includes both a unique label and a shared label.

In some embodiments, the sequencing reads of the target regions may be aligned to corresponding target regions of a reference genome (e.g., GRCh37) (e.g., using the Bismark software). The methylation level of the target region may be compared to a methylation level of a target region of anormal sample or pool of normal samples to identify hypomethylated target regions and hypermethylated target regions (i.e., differentially methylated regions). This process may be facilitated through the use of a methylation calling program such as Metilene. Each of the hypomethylated regions and the hypermethylated regions may be examined separately for the presence or absence of a cancer using the machine learning model.

TABLE 1
Exemplary target regions analyzed for methylation patterns. Target regions
correspond to chromosomes, start, and stop positions corresponding to the
human reference genome GRCh37 (UCSC version hg19; www.genome.ucsc.edu).

| seqnames | start | end | width | seqnames | start | end | width |
|---|---|---|---|---|---|---|---|
| chr1 | 801976 | 802585 | 610 | chr1 | 16847136 | 16847644 | 509 |
| chr1 | 1010527 | 1010677 | 151 | chr1 | 16862057 | 16862203 | 147 |
| chr1 | 1267099 | 1267609 | 511 | chr1 | 16973696 | 16974440 | 745 |
| chr1 | 1514875 | 1515147 | 273 | chr1 | 17019044 | 17019134 | 91 |
| chr1 | 1516298 | 1517281 | 984 | chr1 | 17085829 | 17086764 | 936 |
| chr1 | 1518707 | 1519187 | 481 | chr1 | 17198730 | 17198879 | 150 |
| chr1 | 2005709 | 2006215 | 507 | chr1 | 17215741 | 17215838 | 98 |
| chr1 | 2350156 | 2351084 | 929 | chr1 | 17475708 | 17475766 | 59 |
| chr1 | 2371017 | 2371203 | 187 | chr1 | 18475774 | 18476031 | 258 |
| chr1 | 2394359 | 2394541 | 183 | chr1 | 18518088 | 18518227 | 140 |
| chr1 | 2536696 | 2536944 | 249 | chr1 | 19600364 | 19600818 | 455 |
| chr1 | 2582536 | 2583227 | 692 | chr1 | 20348470 | 20348527 | 58 |
| chr1 | 2699786 | 2700208 | 423 | chr1 | 22940457 | 22940572 | 116 |
| chr1 | 2701399 | 2702713 | 1315 | chr1 | 24927247 | 24927484 | 238 |
| chr1 | 2724552 | 2724699 | 148 | chr1 | 26503778 | 26504195 | 418 |
| chr1 | 2726838 | 2726937 | 100 | chr1 | 27687129 | 27687371 | 243 |
| chr1 | 2731134 | 2732409 | 1276 | chr1 | 27961334 | 27961833 | 500 |
| chr1 | 2764423 | 2764780 | 358 | chr1 | 29101819 | 29101953 | 135 |
| chr1 | 2768553 | 2772285 | 3733 | chr1 | 29585898 | 29586049 | 152 |
| chr1 | 2777600 | 2778767 | 1168 | chr1 | 29587088 | 29587211 | 124 |
| chr1 | 2814552 | 2814945 | 394 | chr1 | 33772217 | 33773032 | 816 |
| chr1 | 2844654 | 2845237 | 584 | chr1 | 34642396 | 34642563 | 168 |
| chr1 | 2890286 | 2890774 | 489 | chr1 | 36042650 | 36042714 | 65 |
| chr1 | 2896574 | 2896779 | 206 | chr1 | 36786270 | 36786750 | 481 |
| chr1 | 2904356 | 2904956 | 601 | chr1 | 37543949 | 37544039 | 91 |
| chr1 | 2917853 | 2917935 | 83 | chr1 | 38601882 | 38601986 | 105 |
| chr1 | 2920143 | 2920862 | 720 | chr1 | 38757850 | 38758056 | 207 |
| chr1 | 2948396 | 2948556 | 161 | chr1 | 39025033 | 39025401 | 369 |
| chr1 | 2954851 | 2955158 | 308 | chr1 | 41283878 | 41283933 | 56 |
| chr1 | 2971112 | 2971409 | 298 | chr1 | 42248900 | 42249184 | 285 |
| chr1 | 3336019 | 3336611 | 593 | chr1 | 43473076 | 43473262 | 187 |
| chr1 | 3342248 | 3342308 | 61 | chr1 | 44015888 | 44016072 | 185 |
| chr1 | 3351420 | 3352477 | 1058 | chr1 | 48347647 | 48347984 | 338 |
| chr1 | 3453077 | 3453222 | 146 | chr1 | 48449960 | 48450136 | 177 |
| chr1 | 3493819 | 3494851 | 1033 | chr1 | 55013976 | 55014014 | 39 |
| chr1 | 3628403 | 3628549 | 147 | chr1 | 55266353 | 55266404 | 52 |
| chr1 | 4108312 | 4108516 | 205 | chr1 | 56839946 | 56840232 | 287 |
| chr1 | 4587931 | 4588207 | 277 | chr1 | 56972083 | 56972248 | 166 |
| chr1 | 4771462 | 4771877 | 416 | chr1 | 59041949 | 59042151 | 203 |
| chr1 | 5727859 | 5728190 | 332 | chr1 | 60539281 | 60539676 | 396 |
| chr1 | 6086356 | 6086465 | 110 | chr1 | 61369405 | 61369504 | 100 |
| chr1 | 7022022 | 7022308 | 28 | chr1 | 61509126 | 61509329 | 204 |
| chr1 | 7788281 | 7788375 | 95 | chr1 | 62660741 | 62661449 | 709 |
| chr1 | 9686728 | 9686898 | 171 | chr1 | 67773923 | 67774067 | 145 |
| chr1 | 9777521 | 9777670 | 150 | chr1 | 69778283 | 69778400 | 118 |
| chr1 | 11543084 | 11543259 | 176 | chr1 | 75590357 | 75590533 | 177 |
| chr1 | 11561539 | 11561875 | 337 | chr1 | 78511016 | 78511367 | 352 |
| chr1 | 16163755 | 16164110 | 356 | chr1 | 79472282 | 79472484 | 203 |
| chr1 | 16553398 | 16554570 | 1173 | chr1 | 86081894 | 86082003 | 110 |
| chr1 | 87597988 | 87598163 | 176 | chr1 | 152941145 | 152942638 | 1494 |
| chr1 | 91192669 | 91192749 | 81 | chr1 | 152954209 | 152954881 | 673 |
| chr1 | 91300213 | 91301872 | 1660 | chr1 | 153021537 | 153023049 | 1513 |
| chr1 | 91306649 | 91307375 | 727 | chr1 | 153074789 | 153076084 | 1296 |
| chr1 | 106623481 | 106623854 | 374 | chr1 | 153085391 | 153085970 | 580 |
| chr1 | 110254603 | 110254855 | 253 | chr1 | 153111954 | 153113119 | 1166 |
| chr1 | 110610268 | 110612965 | 2698 | chr1 | 153124255 | 153126099 | 1845 |
| chr1 | 110626210 | 110626397 | 188 | chr1 | 153127975 | 153129057 | 1083 |
| chr1 | 110673294 | 110673377 | 84 | chr1 | 153173324 | 153174952 | 1629 |
| chr1 | 110713711 | 110714334 | 624 | chr1 | 153177315 | 153179342 | 2028 |
| chr1 | 111862962 | 111863056 | 95 | chr1 | 153214295 | 153214385 | 91 |
| chr1 | 112792573 | 112792852 | 280 | chr1 | 153219533 | 153221166 | 1634 |
| chr1 | 115351387 | 115351672 | 286 | chr1 | 153271682 | 153272395 | 714 |
| chr1 | 115396277 | 115396366 | 90 | chr1 | 153363020 | 153363265 | 246 |
| chr1 | 142541541 | 142542519 | 979 | chr1 | 153415893 | 153417622 | 1730 |
| chr1 | 142949465 | 142949860 | 396 | chr1 | 154056756 | 154057506 | 751 |
| chr1 | 146649627 | 146649768 | 142 | chr1 | 154942635 | 154942847 | 213 |
| chr1 | 148891075 | 148892034 | 960 | chr1 | 156357802 | 156357906 | 105 |
| chr1 | 149066854 | 149067746 | 893 | chr1 | 156616822 | 156616884 | 63 |
| chr1 | 149069611 | 149069726 | 116 | chr1 | 156814884 | 156815839 | 956 |
| chr1 | 149100742 | 149101547 | 806 | chr1 | 156862839 | 156863063 | 225 |
| chr1 | 149286462 | 149286525 | 64 | chr1 | 157510748 | 157512513 | 1766 |
| chr1 | 150123610 | 150123735 | 126 | chr1 | 157593433 | 157595501 | 2069 |
| chr1 | 151168534 | 151168653 | 120 | chr1 | 157614064 | 157615540 | 1477 |
| chr1 | 151777821 | 151777881 | 61 | chr1 | 157672779 | 157673481 | 703 |
| chr1 | 151811364 | 151812525 | 1162 | chr1 | 157685436 | 157686726 | 1291 |
| chr1 | 151952861 | 151953770 | 910 | chr1 | 157708693 | 157709267 | 575 |
| chr1 | 152051816 | 152053788 | 1973 | chr1 | 157768238 | 157769037 | 800 |
| chr1 | 152097870 | 152099424 | 1555 | chr1 | 157770901 | 157773030 | 2130 |
| chr1 | 152352142 | 152353025 | 884 | chr1 | 157777195 | 157779332 | 2138 |
| chr1 | 152474584 | 152478371 | 3788 | chr1 | 157793427 | 157794803 | 1377 |
| chr1 | 152480853 | 152481355 | 503 | chr1 | 157803101 | 157805860 | 2760 |
| chr1 | 152483822 | 152484744 | 923 | chr1 | 157866635 | 157867871 | 1237 |
| chr1 | 152516866 | 152519120 | 2255 | chr1 | 157920297 | 157920891 | 595 |
| chr1 | 152552237 | 152553030 | 794 | chr1 | 158325254 | 158325709 | 456 |
| chr1 | 152595299 | 152595447 | 149 | chr1 | 158332476 | 158334891 | 2416 |
| chr1 | 152785066 | 152786262 | 1197 | chr1 | 158402593 | 158403371 | 779 |
| chr1 | 152798086 | 152800255 | 2170 | chr1 | 158414990 | 158415544 | 555 |
| chr1 | 152803161 | 152804507 | 1347 | chr1 | 158517099 | 158517850 | 752 |
| chr1 | 152833076 | 152834403 | 1328 | chr1 | 158532613 | 158533226 | 614 |
| chr1 | 152871893 | 152872973 | 1081 | chr1 | 158540183 | 158540751 | 569 |
| chr1 | 152877126 | 152877897 | 772 | chr1 | 158557399 | 158557613 | 215 |
| chr1 | 152880108 | 152881816 | 1709 | chr1 | 158605349 | 158607043 | 1695 |
| chr1 | 152884104 | 152885508 | 1405 | chr1 | 158622936 | 158623119 | 184 |
| chr1 | 152912278 | 152913909 | 1632 | chr1 | 158686790 | 158687453 | 664 |
| chr1 | 152919374 | 152920173 | 800 | chr1 | 158721020 | 158721601 | 582 |
| chr1 | 152925940 | 152926847 | 908 | chr1 | 158735753 | 158736313 | 561 |
| chr1 | 152933001 | 152934058 | 1058 | chr1 | 158763840 | 158764784 | 945 |
| chr1 | 152937510 | 152939899 | 2390 | chr1 | 158848682 | 158851190 | 2509 |
| chr1 | 158922021 | 158922124 | 104 | chr1 | 235054187 | 235054339 | 153 |
| chr1 | 158931879 | 158932249 | 371 | chr1 | 235147646 | 235147799 | 154 |
| chr1 | 159544225 | 159544580 | 356 | chr1 | 245906284 | 245906601 | 318 |
| chr1 | 160450785 | 160450986 | 202 | chr1 | 248609183 | 248610769 | 1587 |
| chr1 | 160566115 | 160567579 | 1465 | chr1 | 248834819 | 248835228 | 410 |
| chr1 | 160582723 | 160584088 | 1366 | chr2 | 263401 | 263682 | 282 |
| chr1 | 160719819 | 160721030 | 1212 | chr2 | 568115 | 568226 | 112 |
| chr1 | 160870840 | 160871157 | 318 | chr2 | 636631 | 636852 | 222 |
| chr1 | 161450961 | 161452124 | 1164 | chr2 | 2581238 | 2581484 | 247 |
| chr1 | 161535892 | 161535938 | 47 | chr2 | 3045773 | 3046139 | 367 |
| chr1 | 175474321 | 175474472 | 152 | chr2 | 3077028 | 3077199 | 172 |
| chr1 | 178456062 | 178456107 | 46 | chr2 | 3286338 | 3286461 | 124 |
| chr1 | 178991159 | 178991478 | 320 | chr2 | 3653253 | 3653357 | 105 |
| chr1 | 181451336 | 181451497 | 162 | chr2 | 3750575 | 3751125 | 551 |
| chr1 | 182025816 | 182026466 | 651 | chr2 | 5866197 | 5866471 | 275 |
| chr1 | 182921931 | 182922226 | 296 | chr2 | 7571040 | 7571565 | 526 |
| chr1 | 183155444 | 183155526 | 83 | chr2 | 8272684 | 8272806 | 123 |
| chr1 | 201252862 | 201253260 | 399 | chr2 | 8723903 | 8724182 | 280 |
| chr1 | 201665422 | 201665779 | 358 | chr2 | 9352988 | 9353226 | 239 |
| chr1 | 203044797 | 203045058 | 262 | chr2 | 10146593 | 10146827 | 235 |
| chr1 | 203055340 | 203055480 | 141 | chr2 | 10182570 | 10182725 | 156 |
| chr1 | 203258714 | 203258909 | 196 | chr2 | 10231587 | 10231657 | 71 |
| chr1 | 203641598 | 203641775 | 178 | chr2 | 10572107 | 10572202 | 96 |
| chr1 | 203830001 | 203830241 | 241 | chr2 | 10864032 | 10864224 | 193 |
| chr1 | 207378350 | 207378522 | 173 | chr2 | 11029998 | 11030370 | 373 |
| chr1 | 207622299 | 207622397 | 99 | chr2 | 11817654 | 11817818 | 165 |
| chr1 | 207645615 | 207646474 | 860 | chr2 | 20306523 | 20306804 | 282 |
| chr1 | 207663822 | 207665183 | 1362 | chr2 | 24397675 | 24398003 | 329 |
| chr1 | 207999594 | 208000159 | 566 | chr2 | 24677302 | 24677451 | 150 |
| chr1 | 209404923 | 209405316 | 394 | chr2 | 25149511 | 25149598 | 88 |
| chr1 | 209978978 | 209979394 | 417 | chr2 | 25354304 | 25355105 | 802 |
| chr1 | 210052903 | 210053177 | 275 | chr2 | 25384203 | 25384489 | 287 |
| chr1 | 211652449 | 211652498 | 50 | chr2 | 27665141 | 27665718 | 578 |
| chr1 | 214158540 | 214159579 | 1040 | chr2 | 29751851 | 29752117 | 267 |
| chr1 | 214398397 | 214398557 | 161 | chr2 | 33500893 | 33501024 | 132 |
| chr1 | 217312926 | 217313596 | 671 | chr2 | 39187088 | 39187334 | 247 |
| chr1 | 221050115 | 221050896 | 782 | chr2 | 39892291 | 39892371 | 81 |
| chr1 | 223514574 | 223514746 | 173 | chr2 | 42719825 | 42720202 | 378 |
| chr1 | 224372287 | 224372552 | 266 | chr2 | 43328229 | 43328396 | 168 |
| chr1 | 225632646 | 225633081 | 436 | chr2 | 45028158 | 45028596 | 439 |
| chr1 | 226075206 | 226075449 | 244 | chr2 | 45156760 | 45157667 | 908 |
| chr1 | 226099167 | 226099220 | 54 | chr2 | 45159946 | 45159999 | 54 |
| chr1 | 226127168 | 226127478 | 311 | chr2 | 45166974 | 45167493 | 520 |
| chr1 | 227747366 | 227747498 | 133 | chr2 | 45176480 | 45176512 | 33 |
| chr1 | 227748547 | 227749217 | 671 | chr2 | 45181105 | 45182015 | 911 |
| chr1 | 228400157 | 228400472 | 316 | chr2 | 45227520 | 45228192 | 673 |
| chr1 | 228785987 | 228786173 | 187 | chr2 | 45231032 | 45233529 | 2498 |
| chr1 | 230272306 | 230272509 | 204 | chr2 | 45240265 | 45240626 | 362 |
| chr1 | 233751048 | 233751178 | 131 | chr2 | 45397776 | 45398161 | 386 |
| chr2 | 47597062 | 47597193 | 132 | chr2 | 99528949 | 99529342 | 394 |
| chr2 | 54936457 | 54936841 | 385 | chr2 | 100900797 | 100900874 | 78 |
| chr2 | 60591692 | 60592003 | 312 | chr2 | 101095630 | 101095800 | 171 |
| chr2 | 60880271 | 60880456 | 186 | chr2 | 101271878 | 101272323 | 446 |
| chr2 | 62422356 | 62422480 | 125 | chr2 | 103831366 | 103831540 | 175 |
| chr2 | 63279026 | 63280871 | 1846 | chr2 | 103882575 | 103882797 | 223 |
| chr2 | 65085181 | 65085438 | 258 | chr2 | 104257534 | 104258928 | 1395 |
| chr2 | 65867274 | 65867614 | 341 | chr2 | 104643300 | 104643633 | 334 |
| chr2 | 66672841 | 66673340 | 500 | chr2 | 104924331 | 104925380 | 1050 |
| chr2 | 66809553 | 66809986 | 434 | chr2 | 104942265 | 104942856 | 592 |
| chr2 | 70880838 | 70881421 | 584 | chr2 | 105119591 | 105119954 | 364 |
| chr2 | 71131201 | 71132203 | 1003 | chr2 | 105275689 | 105275728 | 40 |
| chr2 | 71166194 | 71166387 | 194 | chr2 | 105492956 | 105493322 | 367 |
| chr2 | 71287841 | 71288857 | 1017 | chr2 | 105501247 | 105501444 | 198 |
| chr2 | 72058203 | 72058609 | 407 | chr2 | 105587153 | 105588471 | 1319 |
| chr2 | 73551532 | 73551740 | 209 | chr2 | 105735626 | 105735677 | 52 |
| chr2 | 74010609 | 74010682 | 74 | chr2 | 105853537 | 105853620 | 84 |
| chr2 | 74357594 | 74357960 | 367 | chr2 | 110063719 | 110063861 | 143 |
| chr2 | 74642028 | 74642533 | 506 | chr2 | 111876060 | 111876175 | 116 |
| chr2 | 74729710 | 74729885 | 176 | chr2 | 112939121 | 112939540 | 420 |
| chr2 | 74875093 | 74875505 | 413 | chr2 | 119591733 | 119592012 | 280 |
| chr2 | 80549628 | 80549960 | 333 | chr2 | 119594617 | 119594767 | 151 |
| chr2 | 85483237 | 85483374 | 138 | chr2 | 121200188 | 121200964 | 777 |
| chr2 | 85830951 | 85831008 | 58 | chr2 | 121279843 | 121279900 | 58 |
| chr2 | 87036649 | 87036900 | 252 | chr2 | 121310848 | 121311000 | 153 |
| chr2 | 87615063 | 87615191 | 129 | chr2 | 121344587 | 121345341 | 755 |
| chr2 | 88747485 | 88747746 | 262 | chr2 | 121411984 | 121412469 | 486 |
| chr2 | 89254151 | 89256172 | 2022 | chr2 | 121776238 | 121776521 | 284 |
| chr2 | 89269059 | 89269866 | 808 | chr2 | 123418757 | 123419149 | 393 |
| chr2 | 89297366 | 89297632 | 267 | chr2 | 128050901 | 128050977 | 77 |
| chr2 | 89880308 | 89881651 | 1344 | chr2 | 129251165 | 129251285 | 121 |
| chr2 | 89889209 | 89890426 | 1218 | chr2 | 130345057 | 130345229 | 173 |
| chr2 | 91683009 | 91683162 | 154 | chr2 | 131745562 | 131745805 | 244 |
| chr2 | 91693675 | 91693862 | 188 | chr2 | 131797392 | 131798088 | 697 |
| chr2 | 91901018 | 91901639 | 622 | chr2 | 132481660 | 132481931 | 272 |
| chr2 | 91920145 | 91920901 | 757 | chr2 | 132587118 | 132587265 | 148 |
| chr2 | 91926959 | 91927255 | 297 | chr2 | 133014149 | 133014364 | 216 |
| chr2 | 91943712 | 91944114 | 403 | chr2 | 134785419 | 134785829 | 411 |
| chr2 | 91968501 | 91968609 | 109 | chr2 | 150176881 | 150177200 | 320 |
| chr2 | 92032405 | 92033091 | 687 | chr2 | 162101201 | 162101702 | 502 |
| chr2 | 92034536 | 92035168 | 633 | chr2 | 162284394 | 162284695 | 302 |
| chr2 | 92089996 | 92090624 | 629 | chr2 | 164204741 | 164204798 | 58 |
| chr2 | 92146386 | 92146723 | 338 | chr2 | 170624952 | 170625392 | 441 |
| chr2 | 92153284 | 92153655 | 372 | chr2 | 171069760 | 171069885 | 126 |
| chr2 | 95583257 | 95584628 | 1372 | chr2 | 172944997 | 172946623 | 1627 |
| chr2 | 95696498 | 95696819 | 322 | chr2 | 173013101 | 173013301 | 201 |
| chr2 | 95701693 | 95701940 | 248 | chr2 | 173539527 | 173539622 | 96 |
| chr2 | 96781717 | 96781976 | 260 | chr2 | 176931453 | 176933449 | 1997 |
| chr2 | 97505162 | 97505485 | 324 | chr2 | 176936247 | 176937033 | 787 |
| chr2 | 176965401 | 176965497 | 97 | chr3 | 44622433 | 44622808 | 376 |
| chr2 | 177012117 | 177017798 | 5682 | chr3 | 46968623 | 46968900 | 278 |
| chr2 | 177020616 | 177021301 | 686 | chr3 | 48694141 | 48694389 | 249 |
| chr2 | 177024057 | 177026271 | 2215 | chr3 | 49941174 | 49941579 | 406 |
| chr2 | 177027323 | 177029278 | 1956 | chr3 | 49967763 | 49967859 | 97 |
| chr2 | 177036289 | 177037459 | 1171 | chr3 | 50377804 | 50378008 | 205 |
| chr2 | 180293877 | 180294114 | 238 | chr3 | 50395714 | 50395879 | 166 |
| chr2 | 191625846 | 191626109 | 264 | chr3 | 50539360 | 50539512 | 153 |
| chr2 | 192711197 | 192711442 | 246 | chr3 | 51989769 | 51989834 | 66 |
| chr2 | 198029019 | 198029391 | 373 | chr3 | 54277276 | 54277805 | 530 |
| chr2 | 205180018 | 205180245 | 228 | chr3 | 54671901 | 54673633 | 1733 |
| chr2 | 206744268 | 206744340 | 73 | chr3 | 56950280 | 56950403 | 124 |
| chr2 | 208575239 | 208575276 | 38 | chr3 | 58572555 | 58572761 | 207 |
| chr2 | 208631515 | 208631997 | 483 | chr3 | 62362776 | 62363082 | 307 |
| chr2 | 208635465 | 208636041 | 577 | chr3 | 72397196 | 72397257 | 62 |
| chr2 | 208908797 | 208908870 | 74 | chr3 | 72704324 | 72704544 | 221 |
| chr2 | 208976953 | 208977191 | 239 | chr3 | 98125588 | 98126128 | 541 |
| chr2 | 218767430 | 218767637 | 208 | chr3 | 107416645 | 107416777 | 133 |
| chr2 | 218781650 | 218781815 | 166 | chr3 | 113417935 | 113418205 | 271 |
| chr2 | 218898584 | 218898835 | 252 | chr3 | 120025481 | 120025612 | 132 |
| chr2 | 220312798 | 220313866 | 1069 | chr3 | 121898606 | 121898874 | 269 |
| chr2 | 220348259 | 220349742 | 1484 | chr3 | 123602520 | 123602636 | 117 |
| chr2 | 220361549 | 220362018 | 470 | chr3 | 125477819 | 125477906 | 88 |
| chr2 | 228482397 | 228482462 | 66 | chr3 | 125819802 | 125819915 | 114 |
| chr2 | 232055025 | 232055380 | 356 | chr3 | 126007332 | 126007513 | 182 |
| chr2 | 232348597 | 232348849 | 253 | chr3 | 127634352 | 127634460 | 109 |
| chr2 | 236100615 | 236100697 | 83 | chr3 | 128372247 | 128372637 | 391 |
| chr2 | 237067929 | 237069871 | 1943 | chr3 | 130446125 | 130446314 | 190 |
| chr2 | 237071846 | 237072327 | 482 | chr3 | 131080444 | 131080967 | 524 |
| chr2 | 237123097 | 237123123 | 27 | chr3 | 134749886 | 134749937 | 52 |
| chr2 | 239925096 | 239925448 | 353 | chr3 | 137228141 | 137228394 | 254 |
| chr2 | 240196828 | 240197148 | 321 | chr3 | 147108916 | 147108971 | 56 |
| chr2 | 240970850 | 240971713 | 864 | chr3 | 156838174 | 156838410 | 237 |
| chr2 | 240983050 | 240983982 | 933 | chr3 | 157260684 | 157260733 | 50 |
| chr2 | 240996294 | 240996750 | 457 | chr3 | 157811992 | 157814024 | 2033 |
| chr2 | 241029919 | 241030858 | 940 | chr3 | 157820597 | 157820669 | 73 |
| chr2 | 241044715 | 241045493 | 779 | chr3 | 157821987 | 157822073 | 87 |
| chr2 | 241047417 | 241048235 | 819 | chr3 | 158389871 | 158390672 | 802 |
| chr2 | 241845738 | 241846324 | 587 | chr3 | 160203373 | 160203472 | 100 |
| chr2 | 242491745 | 242491793 | 49 | chr3 | 183621711 | 183621849 | 139 |
| chr2 | 242785092 | 242785193 | 102 | chr3 | 183959318 | 183959370 | 53 |
| chr3 | 10651122 | 10651206 | 85 | chr3 | 185911383 | 185912391 | 1009 |
| chr3 | 11178593 | 11178961 | 369 | chr3 | 192289202 | 192289264 | 63 |
| chr3 | 15687681 | 15687951 | 271 | chr3 | 193987466 | 193987707 | 242 |
| chr3 | 18411275 | 18411439 | 165 | chr3 | 194117838 | 194118399 | 562 |
| chr3 | 25614970 | 25615215 | 246 | chr3 | 194705828 | 194705951 | 124 |
| chr3 | 26666659 | 26666771 | 113 | chr3 | 195943084 | 195943986 | 903 |
| chr3 | 29482162 | 29482290 | 129 | chr4 | 651416 | 652067 | 652 |
| chr3 | 30537594 | 30537986 | 393 | chr4 | 659985 | 660206 | 222 |
| chr4 | 779053 | 779122 | 70 | chr4 | 113432509 | 113432576 | 68 |
| chr4 | 1046863 | 1047624 | 762 | chr4 | 129308148 | 129308390 | 243 |
| chr4 | 1406466 | 1406637 | 172 | chr4 | 141217536 | 141217769 | 234 |
| chr4 | 1413180 | 1413323 | 144 | chr4 | 152329051 | 152329318 | 268 |
| chr4 | 1494580 | 1495060 | 481 | chr4 | 154126325 | 154126521 | 197 |
| chr4 | 1522034 | 1522346 | 313 | chr4 | 155411178 | 155411559 | 382 |
| chr4 | 1535666 | 1535705 | 40 | chr4 | 165634846 | 165635203 | 358 |
| chr4 | 1556846 | 1556970 | 125 | chr4 | 166850282 | 166850473 | 192 |
| chr4 | 2765561 | 2765685 | 125 | chr4 | 170695932 | 170696087 | 156 |
| chr4 | 2800685 | 2800764 | 80 | chr4 | 174421206 | 174423369 | 2164 |
| chr4 | 2807568 | 2807971 | 404 | chr4 | 174428089 | 174429105 | 1017 |
| chr4 | 3387141 | 3387342 | 202 | chr4 | 183061792 | 183063465 | 1674 |
| chr4 | 4117424 | 4118084 | 661 | chr4 | 184018892 | 184019081 | 190 |
| chr4 | 4172308 | 4172461 | 154 | chr4 | 184908857 | 184909012 | 156 |
| chr4 | 4408327 | 4408771 | 445 | chr4 | 185268636 | 185270000 | 1365 |
| chr4 | 4410067 | 4410132 | 66 | chr4 | 187219378 | 187219636 | 259 |
| chr4 | 4671079 | 4671120 | 42 | chr4 | 188916529 | 188917068 | 540 |
| chr4 | 5879190 | 5879630 | 441 | chr4 | 189204296 | 189204366 | 71 |
| chr4 | 6445076 | 6445212 | 137 | chr4 | 190731897 | 190731989 | 93 |
| chr4 | 8395800 | 8395938 | 139 | chr5 | 104128 | 104398 | 271 |
| chr4 | 8990863 | 8990982 | 120 | chr5 | 588441 | 588741 | 301 |
| chr4 | 8994788 | 8994900 | 113 | chr5 | 958786 | 958866 | 81 |
| chr4 | 9025156 | 9025811 | 656 | chr5 | 978298 | 978470 | 173 |
| chr4 | 9027686 | 9028116 | 431 | chr5 | 1256969 | 1257002 | 34 |
| chr4 | 10325866 | 10327434 | 1569 | chr5 | 1360951 | 1363332 | 2382 |
| chr4 | 11370378 | 11370809 | 432 | chr5 | 1390831 | 1391310 | 480 |
| chr4 | 39448464 | 39449023 | 560 | chr5 | 1395698 | 1395873 | 176 |
| chr4 | 40632432 | 40632459 | 28 | chr5 | 1417408 | 1418052 | 645 |
| chr4 | 42957469 | 42957933 | 465 | chr5 | 1420263 | 1421755 | 1493 |
| chr4 | 44475798 | 44476319 | 522 | chr5 | 1424127 | 1424268 | 142 |
| chr4 | 48493204 | 48493459 | 256 | chr5 | 1522276 | 1522483 | 208 |
| chr4 | 52943043 | 52943198 | 156 | chr5 | 1548414 | 1548724 | 311 |
| chr4 | 54960232 | 54960463 | 232 | chr5 | 1948875 | 1949030 | 156 |
| chr4 | 54961733 | 54961881 | 149 | chr5 | 2018669 | 2018973 | 305 |
| chr4 | 54975250 | 54976167 | 918 | chr5 | 2442652 | 2443119 | 468 |
| chr4 | 56023880 | 56024003 | 124 | chr5 | 2703282 | 2703427 | 146 |
| chr4 | 56659674 | 56660129 | 456 | chr5 | 2847877 | 2848032 | 156 |
| chr4 | 58060670 | 58060810 | 141 | chr5 | 2866620 | 2867525 | 906 |
| chr4 | 62383028 | 62383200 | 173 | chr5 | 3599343 | 3600262 | 920 |
| chr4 | 81128230 | 81128709 | 480 | chr5 | 3602487 | 3602559 | 73 |
| chr4 | 85402250 | 85403846 | 1597 | chr5 | 3605783 | 3606565 | 783 |
| chr4 | 85405164 | 85405387 | 224 | chr5 | 5183447 | 5183512 | 66 |
| chr4 | 85417812 | 85417911 | 100 | chr5 | 5340446 | 5340649 | 204 |
| chr4 | 92934586 | 92934789 | 204 | chr5 | 7395169 | 7395396 | 228 |
| chr4 | 102711842 | 102712524 | 683 | chr5 | 10333413 | 10333492 | 80 |
| chr4 | 103534054 | 103534327 | 274 | chr5 | 10563077 | 10563317 | 241 |
| chr4 | 108642411 | 108642618 | 208 | chr5 | 14440798 | 14441345 | 548 |
| chr4 | 109291581 | 109291681 | 101 | chr5 | 17402318 | 17402607 | 290 |
| chr4 | 111555191 | 111555216 | 26 | chr5 | 35993394 | 35993481 | 88 |
| chr5 | 38473149 | 38473320 | 172 | chr5 | 140188208 | 140188450 | 243 |
| chr5 | 39425954 | 39426297 | 344 | chr5 | 140202635 | 140203475 | 841 |
| chr5 | 40756365 | 40756441 | 77 | chr5 | 140208845 | 140209923 | 1079 |
| chr5 | 43332139 | 43332946 | 808 | chr5 | 140214244 | 140216005 | 1762 |
| chr5 | 50673201 | 50673793 | 593 | chr5 | 140222139 | 140223066 | 928 |
| chr5 | 50695648 | 50696058 | 411 | chr5 | 140228203 | 140230294 | 2092 |
| chr5 | 66462290 | 66462581 | 292 | chr5 | 140235957 | 140237841 | 1885 |
| chr5 | 67483495 | 67483593 | 99 | chr5 | 140242236 | 140242937 | 702 |
| chr5 | 68710858 | 68711452 | 595 | chr5 | 140250026 | 140250917 | 892 |
| chr5 | 72415570 | 72415671 | 102 | chr5 | 140255227 | 140257259 | 2033 |
| chr5 | 72526839 | 72526965 | 127 | chr5 | 140263154 | 140264085 | 932 |
| chr5 | 72676480 | 72678421 | 1942 | chr5 | 140475709 | 140476558 | 850 |
| chr5 | 77147362 | 77147388 | 27 | chr5 | 140481559 | 140482418 | 860 |
| chr5 | 77253527 | 77253607 | 81 | chr5 | 140502924 | 140503804 | 881 |
| chr5 | 77269213 | 77269456 | 244 | chr5 | 140516508 | 140517041 | 534 |
| chr5 | 80529067 | 80529200 | 134 | chr5 | 140531242 | 140531967 | 726 |
| chr5 | 81653010 | 81653164 | 155 | chr5 | 140537167 | 140537752 | 586 |
| chr5 | 87299727 | 87300313 | 587 | chr5 | 140554006 | 140554507 | 502 |
| chr5 | 92906264 | 92907258 | 995 | chr5 | 140559363 | 140559521 | 159 |
| chr5 | 92938071 | 92938186 | 116 | chr5 | 140563400 | 140564231 | 832 |
| chr5 | 112395122 | 112395399 | 278 | chr5 | 140568487 | 140569054 | 568 |
| chr5 | 114514861 | 114514995 | 135 | chr5 | 140573721 | 140574294 | 574 |
| chr5 | 119794016 | 119794137 | 122 | chr5 | 140580728 | 140581482 | 755 |
| chr5 | 120038336 | 120038374 | 39 | chr5 | 140589820 | 140590642 | 823 |
| chr5 | 122431419 | 122431539 | 121 | chr5 | 140595314 | 140595792 | 479 |
| chr5 | 122433895 | 122434141 | 247 | chr5 | 140604406 | 140605248 | 843 |
| chr5 | 128626360 | 128626713 | 354 | chr5 | 140615656 | 140616353 | 698 |
| chr5 | 131594903 | 131595195 | 293 | chr5 | 140621072 | 140621603 | 532 |
| chr5 | 131706724 | 131706765 | 42 | chr5 | 140626460 | 140627258 | 799 |
| chr5 | 131802182 | 131802313 | 132 | chr5 | 140683502 | 140683759 | 258 |
| chr5 | 132083532 | 132083751 | 220 | chr5 | 140712137 | 140712478 | 342 |
| chr5 | 132150162 | 132150278 | 117 | chr5 | 140718798 | 140718933 | 136 |
| chr5 | 132158798 | 132158867 | 70 | chr5 | 140723651 | 140723816 | 166 |
| chr5 | 133513514 | 133513652 | 139 | chr5 | 140731195 | 140731933 | 739 |
| chr5 | 134363053 | 134364975 | 1923 | chr5 | 140734911 | 140735432 | 522 |
| chr5 | 134376475 | 134376919 | 445 | chr5 | 140741195 | 140742043 | 849 |
| chr5 | 134385970 | 134386282 | 313 | chr5 | 140743791 | 140746137 | 2347 |
| chr5 | 134825466 | 134826939 | 1474 | chr5 | 140749949 | 140752102 | 2154 |
| chr5 | 135313196 | 135313351 | 156 | chr5 | 140753536 | 140753925 | 390 |
| chr5 | 138923113 | 138923219 | 107 | chr5 | 140755402 | 140755984 | 583 |
| chr5 | 139737779 | 139737927 | 149 | chr5 | 140762704 | 140763142 | 439 |
| chr5 | 140045248 | 140045362 | 115 | chr5 | 140768940 | 140769592 | 653 |
| chr5 | 140052204 | 140052244 | 41 | chr5 | 140772311 | 140774661 | 2351 |
| chr5 | 140140179 | 140140643 | 465 | chr5 | 140777488 | 140779829 | 2342 |
| chr5 | 140155785 | 140155965 | 181 | chr5 | 140782510 | 140783199 | 690 |
| chr5 | 140167108 | 140167967 | 860 | chr5 | 140787504 | 140789798 | 2295 |
| chr5 | 140175725 | 140176250 | 526 | chr5 | 140792684 | 140795043 | 2360 |
| chr5 | 140181058 | 140181121 | 64 | chr5 | 140797398 | 140799544 | 2147 |
| chr5 | 140182209 | 140182973 | 765 | chr5 | 140800686 | 140803131 | 2446 |
| chr5 | 140807137 | 140807925 | 789 | chr6 | 17016237 | 17016563 | 327 |
| chr5 | 140810137 | 140812691 | 2555 | chr6 | 17200762 | 17200958 | 197 |
| chr5 | 140821313 | 140821688 | 376 | chr6 | 18122474 | 18122546 | 73 |
| chr5 | 140870948 | 140872467 | 1520 | chr6 | 21667558 | 21667971 | 414 |
| chr5 | 143978070 | 143978240 | 171 | chr6 | 21722971 | 21723030 | 60 |
| chr5 | 146000208 | 146000638 | 431 | chr6 | 22147102 | 22147192 | 91 |
| chr5 | 149546377 | 149547175 | 799 | chr6 | 24357665 | 24358611 | 947 |
| chr5 | 149887114 | 149887592 | 479 | chr6 | 25232816 | 25232882 | 67 |
| chr5 | 149922697 | 149922885 | 189 | chr6 | 26017532 | 26018119 | 588 |
| chr5 | 153901042 | 153901177 | 136 | chr6 | 26240528 | 26240931 | 40 |
| chr5 | 154026413 | 154026841 | 429 | chr6 | 26522270 | 26522306 | 37 |
| chr5 | 154070874 | 154071015 | 142 | chr6 | 27210119 | 27210183 | 65 |
| chr5 | 154151148 | 154151236 | 89 | chr6 | 27222357 | 27222897 | 541 |
| chr5 | 155000434 | 155000792 | 359 | chr6 | 27334720 | 27336121 | 1402 |
| chr5 | 156277365 | 156277543 | 179 | chr6 | 27596310 | 27597040 | 731 |
| chr5 | 157078618 | 157079105 | 488 | chr6 | 27725176 | 27725230 | 55 |
| chr5 | 157098813 | 157098908 | 96 | chr6 | 27831903 | 27832284 | 382 |
| chr5 | 157117740 | 157117819 | 80 | chr6 | 27935040 | 27935289 | 250 |
| chr5 | 158531207 | 158533285 | 2079 | chr6 | 27965685 | 27966644 | 960 |
| chr5 | 159560692 | 159560820 | 129 | chr6 | 27982261 | 27983197 | 937 |
| chr5 | 170741288 | 170741350 | 63 | chr6 | 28018008 | 28018396 | 389 |
| chr5 | 172277008 | 172277192 | 185 | chr6 | 28449579 | 28450576 | 998 |
| chr5 | 175105255 | 175105566 | 312 | chr6 | 28583971 | 28584444 | 474 |
| chr5 | 175488242 | 175488850 | 609 | chr6 | 29315681 | 29315903 | 223 |
| chr5 | 175579246 | 175579448 | 203 | chr6 | 29327463 | 29328144 | 682 |
| chr5 | 176024418 | 176024488 | 71 | chr6 | 29394863 | 29395188 | 326 |
| chr5 | 176107140 | 176107454 | 315 | chr6 | 29407852 | 29408230 | 379 |
| chr5 | 176559249 | 176559721 | 473 | chr6 | 29435173 | 29435449 | 277 |
| chr5 | 176827135 | 176827326 | 192 | chr6 | 30100782 | 30100851 | 70 |
| chr5 | 180591454 | 180591753 | 300 | chr6 | 30372489 | 30373238 | 750 |
| chr5 | 180646417 | 180646525 | 109 | chr6 | 30384617 | 30385055 | 439 |
| chr5 | 180700397 | 180700629 | 233 | chr6 | 30691721 | 30691792 | 72 |
| chr6 | 221171 | 221400 | 230 | chr6 | 31650649 | 31651060 | 412 |
| chr6 | 327501 | 327695 | 195 | chr6 | 32070473 | 32070786 | 314 |
| chr6 | 366645 | 367170 | 526 | chr6 | 32116591 | 32117172 | 582 |
| chr6 | 445012 | 445180 | 169 | chr6 | 32188731 | 32189127 | 397 |
| chr6 | 453072 | 453338 | 267 | chr6 | 33560700 | 33561254 | 555 |
| chr6 | 1111303 | 1111414 | 112 | chr6 | 34202981 | 34204001 | 1021 |
| chr6 | 1601847 | 1601883 | 37 | chr6 | 35002597 | 35002840 | 244 |
| chr6 | 1619830 | 1621024 | 1195 | chr6 | 36098787 | 36099243 | 457 |
| chr6 | 3438580 | 3438803 | 224 | chr6 | 36628459 | 36628883 | 425 |
| chr6 | 4079156 | 4079251 | 96 | chr6 | 37045823 | 37045932 | 110 |
| chr6 | 4607525 | 4607681 | 157 | chr6 | 37593465 | 37593666 | 202 |
| chr6 | 5472047 | 5472379 | 333 | chr6 | 37616284 | 37616522 | 239 |
| chr6 | 5827500 | 5827584 | 85 | chr6 | 37617864 | 37618117 | 254 |
| chr6 | 5851824 | 5851867 | 44 | chr6 | 41528395 | 41528817 | 423 |
| chr6 | 6002032 | 6002892 | 861 | chr6 | 42247341 | 42247711 | 371 |
| chr6 | 13428522 | 13428752 | 231 | chr6 | 42695054 | 42695118 | 65 |
| chr6 | 16216914 | 16217173 | 260 | chr6 | 43091542 | 43091619 | 78 |
| chr6 | 43243974 | 43244295 | 322 | chr6 | 170338360 | 170338749 | 390 |
| chr6 | 43544618 | 43544777 | 160 | chr6 | 170589858 | 170589898 | 41 |
| chr6 | 43957451 | 43958022 | 572 | chr6 | 170732238 | 170732339 | 102 |
| chr6 | 46293409 | 46293863 | 455 | chr7 | 475952 | 476132 | 181 |
| chr6 | 49590649 | 49590798 | 150 | chr7 | 946557 | 946873 | 317 |
| chr6 | 50810909 | 50810961 | 53 | chr7 | 1029198 | 1029846 | 649 |
| chr6 | 50813557 | 50813644 | 88 | chr7 | 1100234 | 1100273 | 40 |
| chr6 | 57180990 | 57181125 | 136 | chr7 | 1224630 | 1224989 | 360 |
| chr6 | 63055041 | 63055987 | 947 | chr7 | 1295867 | 1295920 | 54 |
| chr6 | 63449460 | 63450573 | 1114 | chr7 | 1299977 | 1303495 | 3519 |
| chr6 | 72298331 | 72298481 | 151 | chr7 | 1308471 | 1309985 | 1515 |
| chr6 | 74024484 | 74024818 | 335 | chr7 | 1314267 | 1314349 | 83 |
| chr6 | 74058637 | 74058740 | 104 | chr7 | 1358101 | 1358686 | 586 |
| chr6 | 74233302 | 74233331 | 30 | chr7 | 1370727 | 1370821 | 95 |
| chr6 | 78359926 | 78359964 | 39 | chr7 | 1383304 | 1383387 | 84 |
| chr6 | 85476147 | 85476841 | 695 | chr7 | 1422931 | 1423059 | 129 |
| chr6 | 87832381 | 87832563 | 183 | chr7 | 1428692 | 1428925 | 234 |
| chr6 | 90272372 | 90272587 | 216 | chr7 | 1577993 | 1578091 | 99 |
| chr6 | 90695183 | 90695543 | 361 | chr7 | 1626764 | 1627036 | 273 |
| chr6 | 100897117 | 100897514 | 398 | chr7 | 1686836 | 1686905 | 70 |
| chr6 | 100917402 | 100917459 | 58 | chr7 | 1733546 | 1733865 | 320 |
| chr6 | 105400882 | 105401127 | 246 | chr7 | 2077595 | 2077729 | 135 |
| chr6 | 108488828 | 108488997 | 170 | chr7 | 2571075 | 2571576 | 502 |
| chr6 | 108492269 | 108492708 | 440 | chr7 | 2797050 | 2797291 | 242 |
| chr6 | 112688515 | 112688613 | 99 | chr7 | 2968275 | 2968628 | 354 |
| chr6 | 118103691 | 118104573 | 883 | chr7 | 4304656 | 4305029 | 374 |
| chr6 | 133561660 | 133561736 | 77 | chr7 | 4901367 | 4901751 | 385 |
| chr6 | 134436683 | 134436756 | 74 | chr7 | 5147070 | 5147408 | 339 |
| chr6 | 137244797 | 137245163 | 367 | chr7 | 5167221 | 5167435 | 215 |
| chr6 | 148829731 | 148829908 | 178 | chr7 | 5597707 | 5597813 | 107 |
| chr6 | 150374092 | 150374926 | 835 | chr7 | 5732119 | 5732281 | 163 |
| chr6 | 152126012 | 152126338 | 327 | chr7 | 6388960 | 6389050 | 91 |
| chr6 | 153472269 | 153472466 | 198 | chr7 | 6581220 | 6581381 | 162 |
| chr6 | 155475521 | 155475654 | 134 | chr7 | 23245591 | 23246015 | 425 |
| chr6 | 157204742 | 157204869 | 128 | chr7 | 24436334 | 24436464 | 131 |
| chr6 | 157557974 | 157558097 | 124 | chr7 | 27252842 | 27253179 | 338 |
| chr6 | 158937265 | 158937551 | 287 | chr7 | 27260186 | 27260418 | 233 |
| chr6 | 159256895 | 159257116 | 222 | chr7 | 28995252 | 28995418 | 167 |
| chr6 | 159360259 | 159360921 | 663 | chr7 | 29923410 | 29923833 | 424 |
| chr6 | 160985673 | 160985814 | 142 | chr7 | 30682097 | 30682165 | 69 |
| chr6 | 160992185 | 160993269 | 1085 | chr7 | 32801617 | 32802794 | 1178 |
| chr6 | 161011371 | 161011883 | 513 | chr7 | 34171067 | 34171216 | 150 |
| chr6 | 161015089 | 161015613 | 525 | chr7 | 35294460 | 35295158 | 699 |
| chr6 | 161021992 | 161022567 | 576 | chr7 | 35298018 | 35298665 | 648 |
| chr6 | 161027325 | 161027714 | 390 | chr7 | 35301724 | 35301798 | 75 |
| chr6 | 163818110 | 163818152 | 43 | chr7 | 35494317 | 35494346 | 30 |
| chr6 | 164051583 | 164052181 | 599 | chr7 | 36007356 | 36007458 | 103 |
| chr6 | 166218653 | 166218700 | 48 | chr7 | 36011155 | 36011358 | 204 |
| chr6 | 168502098 | 168502443 | 346 | chr7 | 37025447 | 37025605 | 159 |
| chr7 | 47092303 | 47092640 | 338 | chr7 | 63037876 | 63038270 | 395 |
| chr7 | 50132778 | 50132875 | 98 | chr7 | 63118238 | 63118601 | 364 |
| chr7 | 50218832 | 50219354 | 523 | chr7 | 63300001 | 63300915 | 915 |
| chr7 | 51385085 | 51385366 | 282 | chr7 | 63317786 | 63318326 | 541 |
| chr7 | 55256109 | 55256376 | 268 | chr7 | 63341611 | 63341831 | 221 |
| chr7 | 55516883 | 55517246 | 364 | chr7 | 63346530 | 63347187 | 658 |
| chr7 | 55887904 | 55888462 | 559 | chr7 | 63401401 | 63401927 | 527 |
| chr7 | 56019060 | 56019247 | 188 | chr7 | 63549378 | 63549944 | 567 |
| chr7 | 56285881 | 56286515 | 635 | chr7 | 63556596 | 63557311 | 716 |
| chr7 | 57160279 | 57160615 | 337 | chr7 | 63593039 | 63595163 | 2125 |
| chr7 | 57174455 | 57175215 | 761 | chr7 | 63599144 | 63599767 | 624 |
| chr7 | 57200000 | 57201974 | 1975 | chr7 | 63621281 | 63622315 | 1035 |
| chr7 | 57218855 | 57218968 | 114 | chr7 | 63653326 | 63653742 | 417 |
| chr7 | 57221688 | 57222043 | 356 | chr7 | 63662427 | 63663086 | 660 |
| chr7 | 57251238 | 57251862 | 625 | chr7 | 64349006 | 64350151 | 1146 |
| chr7 | 57253921 | 57254573 | 653 | chr7 | 65508726 | 65508863 | 138 |
| chr7 | 57258202 | 57259248 | 1047 | chr7 | 66206842 | 66207063 | 222 |
| chr7 | 57275304 | 57276979 | 1676 | chr7 | 67319221 | 67319425 | 205 |
| chr7 | 57278049 | 57280608 | 2560 | chr7 | 71563891 | 71564083 | 193 |
| chr7 | 57281694 | 57282930 | 1237 | chr7 | 71912387 | 71912624 | 238 |
| chr7 | 57284363 | 57286526 | 2164 | chr7 | 77669671 | 77669868 | 198 |
| chr7 | 57297088 | 57297731 | 644 | chr7 | 91510577 | 91510851 | 275 |
| chr7 | 57303644 | 57304078 | 435 | chr7 | 92238100 | 92238249 | 150 |
| chr7 | 57312179 | 57313126 | 948 | chr7 | 94953782 | 94953878 | 97 |
| chr7 | 57325912 | 57326363 | 452 | chr7 | 95025683 | 95026160 | 478 |
| chr7 | 57360549 | 57361192 | 644 | chr7 | 96631441 | 96631943 | 503 |
| chr7 | 57382926 | 57383949 | 1024 | chr7 | 98122467 | 98122752 | 286 |
| chr7 | 57402223 | 57404123 | 1901 | chr7 | 98185755 | 98185892 | 138 |
| chr7 | 57416388 | 57416780 | 393 | chr7 | 98298741 | 98299330 | 590 |
| chr7 | 57421326 | 57421504 | 179 | chr7 | 99835252 | 99835398 | 147 |
| chr7 | 57428186 | 57428446 | 261 | chr7 | 100034616 | 100034700 | 85 |
| chr7 | 57497562 | 57498926 | 1365 | chr7 | 100297878 | 100298294 | 417 |
| chr7 | 57610254 | 57610669 | 416 | chr7 | 100551362 | 100553436 | 2075 |
| chr7 | 61064262 | 61064568 | 307 | chr7 | 100666253 | 100666813 | 561 |
| chr7 | 62060544 | 62060783 | 240 | chr7 | 100679374 | 100680371 | 998 |
| chr7 | 62470951 | 62471380 | 430 | chr7 | 100685994 | 100686778 | 785 |
| chr7 | 62484953 | 62486736 | 1784 | chr7 | 100692132 | 100692700 | 569 |
| chr7 | 62490802 | 62492375 | 1574 | chr7 | 100693744 | 100696528 | 2785 |
| chr7 | 62562619 | 62563282 | 664 | chr7 | 100921970 | 100922723 | 754 |
| chr7 | 62646589 | 62646744 | 156 | chr7 | 101943884 | 101944276 | 393 |
| chr7 | 62684323 | 62684962 | 640 | chr7 | 104236344 | 104236992 | 649 |
| chr7 | 62699907 | 62700006 | 100 | chr7 | 105319307 | 105319478 | 172 |
| chr7 | 62766139 | 62766423 | 285 | chr7 | 105711875 | 105712118 | 244 |
| chr7 | 62938203 | 62938384 | 182 | chr7 | 106272550 | 106273371 | 822 |
| chr7 | 62964839 | 62964992 | 154 | chr7 | 111203107 | 111203146 | 40 |
| chr7 | 62981704 | 62982444 | 741 | chr7 | 121472306 | 121472553 | 248 |
| chr7 | 63020222 | 63021252 | 1031 | chr7 | 121504395 | 121504548 | 154 |
| chr7 | 63025762 | 63025949 | 188 | chr7 | 124404242 | 124404304 | 63 |
| chr7 | 63031385 | 63031683 | 299 | chr7 | 127880992 | 127881313 | 322 |
| chr7 | 127910909 | 127912454 | 1546 | chr8 | 21905547 | 21905738 | 192 |
| chr7 | 127991421 | 127991843 | 423 | chr8 | 21944075 | 21944358 | 284 |
| chr7 | 128556021 | 128556361 | 341 | chr8 | 22437459 | 22437701 | 243 |
| chr7 | 130005604 | 130005662 | 59 | chr8 | 22547469 | 22548284 | 816 |
| chr7 | 130125932 | 130126598 | 667 | chr8 | 22875892 | 22876275 | 384 |
| chr7 | 130417992 | 130418194 | 203 | chr8 | 26721677 | 26722205 | 529 |
| chr7 | 137620347 | 137620748 | 402 | chr8 | 27546716 | 27546976 | 261 |
| chr7 | 140103783 | 140104011 | 229 | chr8 | 37238156 | 37238611 | 456 |
| chr7 | 141662323 | 141662981 | 659 | chr8 | 38757677 | 38757977 | 301 |
| chr7 | 141941614 | 141943805 | 2192 | chr8 | 40790252 | 40790494 | 243 |
| chr7 | 149120159 | 149120864 | 706 | chr8 | 41200604 | 41200733 | 130 |
| chr7 | 150655304 | 150655522 | 219 | chr8 | 41424342 | 41425132 | 791 |
| chr7 | 151145247 | 151145413 | 167 | chr8 | 41583136 | 41583524 | 389 |
| chr7 | 151536404 | 151536433 | 30 | chr8 | 41654921 | 41655443 | 523 |
| chr7 | 152621963 | 152622058 | 96 | chr8 | 46941234 | 46941366 | 133 |
| chr7 | 154027410 | 154027790 | 381 | chr8 | 49494204 | 49494233 | 30 |
| chr7 | 154585943 | 154586232 | 290 | chr8 | 53441737 | 53442398 | 662 |
| chr7 | 155754586 | 155754770 | 185 | chr8 | 53730234 | 53731113 | 880 |
| chr7 | 155970166 | 155970261 | 96 | chr8 | 53737726 | 53738896 | 1171 |
| chr7 | 156101749 | 156102413 | 665 | chr8 | 53764869 | 53765748 | 880 |
| chr7 | 156716006 | 156716129 | 124 | chr8 | 53786518 | 53787574 | 1057 |
| chr7 | 156836773 | 156836899 | 127 | chr8 | 53795245 | 53796273 | 1029 |
| chr7 | 157369246 | 157369298 | 53 | chr8 | 53807367 | 53807938 | 572 |
| chr7 | 157478134 | 157478899 | 766 | chr8 | 55359300 | 55361078 | 1779 |
| chr7 | 157776330 | 157776798 | 469 | chr8 | 55429083 | 55429919 | 837 |
| chr7 | 157890121 | 157890783 | 663 | chr8 | 55439610 | 55440194 | 585 |
| chr7 | 157996871 | 157996946 | 76 | chr8 | 55482549 | 55482920 | 372 |
| chr7 | 158032448 | 158032574 | 127 | chr8 | 55499331 | 55499447 | 117 |
| chr7 | 158044839 | 158044957 | 119 | chr8 | 55512731 | 55513567 | 837 |
| chr7 | 158066368 | 158066880 | 513 | chr8 | 55691104 | 55691519 | 416 |
| chr7 | 158362371 | 158362595 | 225 | chr8 | 55761377 | 55761473 | 97 |
| chr7 | 158984072 | 158984283 | 212 | chr8 | 55885034 | 55885629 | 596 |
| chr7 | 159004677 | 159005150 | 474 | chr8 | 55893368 | 55894351 | 984 |
| chr8 | 1649253 | 1649703 | 451 | chr8 | 55898683 | 55899078 | 396 |
| chr8 | 1765039 | 1765789 | 751 | chr8 | 55902200 | 55903267 | 1068 |
| chr8 | 1950784 | 1950838 | 55 | chr8 | 55917887 | 55918396 | 510 |
| chr8 | 2419035 | 2419240 | 206 | chr8 | 55923218 | 55924478 | 1261 |
| chr8 | 6420236 | 6420276 | 41 | chr8 | 55998494 | 55999247 | 754 |
| chr8 | 6877763 | 6878156 | 394 | chr8 | 56331672 | 56332056 | 385 |
| chr8 | 8559842 | 8560278 | 437 | chr8 | 57103508 | 57103834 | 327 |
| chr8 | 9756423 | 9756535 | 113 | chr8 | 57185950 | 57186575 | 626 |
| chr8 | 10709552 | 10709736 | 185 | chr8 | 61835402 | 61835796 | 395 |
| chr8 | 11064899 | 11065359 | 461 | chr8 | 61918247 | 61918451 | 205 |
| chr8 | 11138698 | 11139367 | 670 | chr8 | 65711625 | 65711722 | 98 |
| chr8 | 11247790 | 11247877 | 88 | chr8 | 67838240 | 67838341 | 102 |
| chr8 | 12435291 | 12435960 | 670 | chr8 | 67936672 | 67936948 | 277 |
| chr8 | 12443671 | 12444395 | 725 | chr8 | 68346411 | 68347055 | 645 |
| chr8 | 13016804 | 13016938 | 135 | chr8 | 69188984 | 69189218 | 235 |
| chr8 | 18244740 | 18245210 | 471 | chr8 | 69204772 | 69205139 | 368 |
| chr8 | 70623422 | 70623575 | 154 | chr9 | 118091 | 118159 | 69 |
| chr8 | 72469297 | 72470459 | 1163 | chr9 | 21489083 | 21489483 | 401 |
| chr8 | 74268740 | 74269241 | 502 | chr9 | 21966552 | 21971190 | 4639 |
| chr8 | 76316218 | 76316365 | 148 | chr9 | 21989520 | 21990330 | 811 |
| chr8 | 81478046 | 81478496 | 451 | chr9 | 23823046 | 23823357 | 312 |
| chr8 | 86436353 | 86436685 | 333 | chr9 | 32553335 | 32553409 | 75 |
| chr8 | 86560027 | 86560356 | 330 | chr9 | 32955717 | 32956023 | 307 |
| chr8 | 86572276 | 86572730 | 455 | chr9 | 34379923 | 34380323 | 401 |
| chr8 | 86786346 | 86786746 | 401 | chr9 | 34590689 | 34591345 | 657 |
| chr8 | 90702855 | 90702924 | 70 | chr9 | 35036126 | 35036228 | 103 |
| chr8 | 94508034 | 94508411 | 378 | chr9 | 37787132 | 37787329 | 198 |
| chr8 | 95246369 | 95246963 | 595 | chr9 | 38687709 | 38687768 | 60 |
| chr8 | 99951468 | 99951499 | 32 | chr9 | 39132894 | 39133038 | 145 |
| chr8 | 101821811 | 101822133 | 323 | chr9 | 42253987 | 42254172 | 186 |
| chr8 | 102184389 | 102184453 | 65 | chr9 | 45438209 | 45439849 | 1641 |
| chr8 | 102236239 | 102236458 | 220 | chr9 | 66486994 | 66487048 | 55 |
| chr8 | 102504322 | 102506943 | 2622 | chr9 | 70649479 | 70650131 | 653 |
| chr8 | 105342328 | 105342406 | 79 | chr9 | 87454517 | 87454708 | 192 |
| chr8 | 120844196 | 120844348 | 153 | chr9 | 90487113 | 90487252 | 140 |
| chr8 | 121135425 | 121135522 | 98 | chr9 | 94648806 | 94648899 | 94 |
| chr8 | 124173391 | 124173734 | 344 | chr9 | 96588830 | 96588905 | 76 |
| chr8 | 125984989 | 125985107 | 119 | chr9 | 97094660 | 97095032 | 373 |
| chr8 | 131526692 | 131526870 | 179 | chr9 | 97402255 | 97402501 | 247 |
| chr8 | 133426087 | 133426649 | 563 | chr9 | 97683103 | 97683601 | 499 |
| chr8 | 134419263 | 134419426 | 164 | chr9 | 97818542 | 97818830 | 289 |
| chr8 | 141057225 | 141057380 | 156 | chr9 | 98315391 | 98315538 | 148 |
| chr8 | 141220187 | 141220441 | 255 | chr9 | 98514418 | 98514768 | 351 |
| chr8 | 141599370 | 141599490 | 121 | chr9 | 98949018 | 98949112 | 95 |
| chr8 | 142046261 | 142046398 | 138 | chr9 | 98981374 | 98982062 | 689 |
| chr8 | 142572539 | 142572737 | 199 | chr9 | 100279932 | 100280117 | 186 |
| chr8 | 142893674 | 142893744 | 71 | chr9 | 101159079 | 101159215 | 137 |
| chr8 | 143612209 | 143612428 | 220 | chr9 | 102058712 | 102059153 | 442 |
| chr8 | 143755576 | 143755771 | 196 | chr9 | 109460000 | 109460449 | 450 |
| chr8 | 144153830 | 144154953 | 1124 | chr9 | 112261080 | 112261553 | 474 |
| chr8 | 144163526 | 144164393 | 868 | chr9 | 123834462 | 123834618 | 157 |
| chr8 | 144170090 | 144170809 | 720 | chr9 | 124498486 | 124499143 | 658 |
| chr8 | 144251900 | 144252229 | 330 | chr9 | 125396529 | 125397071 | 543 |
| chr8 | 144461975 | 144462059 | 85 | chr9 | 127105405 | 127105480 | 76 |
| chr8 | 144659913 | 144660635 | 723 | chr9 | 128984864 | 128985437 | 574 |
| chr8 | 144801064 | 144801119 | 56 | chr9 | 129438425 | 129439119 | 695 |
| chr8 | 144827169 | 144827414 | 246 | chr9 | 131012286 | 131012684 | 399 |
| chr8 | 144848172 | 144848537 | 366 | chr9 | 132200859 | 132201089 | 231 |
| chr8 | 145013154 | 145013609 | 456 | chr9 | 132262652 | 132262813 | 162 |
| chr8 | 145101456 | 145102005 | 550 | chr9 | 136047026 | 136047215 | 190 |
| chr8 | 145164544 | 145164694 | 151 | chr9 | 136120905 | 136121209 | 305 |
| chr8 | 145698027 | 145698070 | 44 | chr9 | 136444321 | 136444391 | 71 |
| chr8 | 145806259 | 145806711 | 453 | chr9 | 136568073 | 136568140 | 68 |
| chr8 | 146186621 | 146186793 | 173 | chr9 | 137232664 | 137232914 | 251 |
| chr8 | 146300792 | 146301067 | 276 | chr9 | 137428113 | 137428246 | 134 |
| chr9 | 138039033 | 138039338 | 306 | chr10 | 88728040 | 88728720 | 681 |
| chr9 | 138559376 | 138559538 | 163 | chr10 | 88976000 | 88976120 | 121 |
| chr9 | 138562782 | 138562863 | 82 | chr10 | 89167837 | 89167992 | 156 |
| chr9 | 138575445 | 138575580 | 136 | chr10 | 91596851 | 91597860 | 1010 |
| chr9 | 138688937 | 138689109 | 173 | chr10 | 92616821 | 92616848 | 28 |
| chr9 | 138813015 | 138813237 | 223 | chr10 | 93805513 | 93805758 | 246 |
| chr9 | 138998995 | 138999620 | 626 | chr10 | 96658377 | 96658823 | 447 |
| chr9 | 139062032 | 139062072 | 41 | chr10 | 101261729 | 101261862 | 134 |
| chr9 | 139069816 | 139069898 | 83 | chr10 | 102089882 | 102090224 | 343 |
| chr9 | 139739496 | 139741366 | 1871 | chr10 | 102368765 | 102369289 | 525 |
| chr9 | 139889190 | 139889505 | 316 | chr10 | 102808227 | 102808307 | 81 |
| chr9 | 140324818 | 140324885 | 68 | chr10 | 103533243 | 103533299 | 57 |
| chr9 | 140352619 | 140352841 | 223 | chr10 | 103534451 | 103534525 | 75 |
| chr9 | 140397002 | 140397258 | 257 | chr10 | 105499302 | 105499485 | 184 |
| chr10 | 6183343 | 6183943 | 601 | chr10 | 112838285 | 112839142 | 858 |
| chr10 | 14074531 | 14074625 | 95 | chr10 | 118894394 | 118894478 | 85 |
| chr10 | 15002668 | 15002748 | 81 | chr10 | 118896018 | 118896194 | 177 |
| chr10 | 16561693 | 16561952 | 260 | chr10 | 119494641 | 119494991 | 351 |
| chr10 | 21788975 | 21789510 | 536 | chr10 | 119590450 | 119590574 | 125 |
| chr10 | 22542582 | 22542669 | 88 | chr10 | 119867969 | 119868270 | 302 |
| chr10 | 22622677 | 22623706 | 1030 | chr10 | 120578319 | 120578392 | 74 |
| chr10 | 22765611 | 22766643 | 1033 | chr10 | 121770987 | 121771135 | 149 |
| chr10 | 26391984 | 26392219 | 236 | chr10 | 124581791 | 124582828 | 1038 |
| chr10 | 26926767 | 26927008 | 242 | chr10 | 125444854 | 125445026 | 173 |
| chr10 | 28056534 | 28057533 | 1000 | chr10 | 125650855 | 125650958 | 104 |
| chr10 | 28164982 | 28165578 | 597 | chr10 | 125732783 | 125732824 | 42 |
| chr10 | 31938846 | 31939076 | 231 | chr10 | 126840377 | 126841146 | 770 |
| chr10 | 43271424 | 43271888 | 465 | chr10 | 130064031 | 130064155 | 125 |
| chr10 | 43393644 | 43394272 | 629 | chr10 | 130508368 | 130508655 | 288 |
| chr10 | 43632736 | 43632867 | 132 | chr10 | 133149104 | 133149242 | 139 |
| chr10 | 43698738 | 43698919 | 182 | chr10 | 134235996 | 134236081 | 86 |
| chr10 | 44788235 | 44789011 | 777 | chr10 | 134597683 | 134598300 | 618 |
| chr10 | 49674698 | 49674745 | 48 | chr10 | 134606453 | 134607796 | 1344 |
| chr10 | 50819742 | 50820291 | 550 | chr10 | 134801576 | 134801882 | 307 |
| chr10 | 60086814 | 60087027 | 214 | chr11 | 195107 | 195273 | 167 |
| chr10 | 63627469 | 63627762 | 294 | chr11 | 411066 | 412327 | 1262 |
| chr10 | 72014950 | 72015679 | 730 | chr11 | 1244847 | 1244982 | 136 |
| chr10 | 73565589 | 73565701 | 113 | chr11 | 1252230 | 1253053 | 824 |
| chr10 | 76573175 | 76573455 | 281 | chr11 | 1256758 | 1257119 | 362 |
| chr10 | 76803505 | 76803807 | 303 | chr11 | 1385933 | 1386990 | 1058 |
| chr10 | 77021842 | 77022002 | 161 | chr11 | 1760727 | 1761180 | 454 |
| chr10 | 77035993 | 77036373 | 381 | chr11 | 2473770 | 2473898 | 129 |
| chr10 | 77155473 | 77157173 | 1701 | chr11 | 2890190 | 2890967 | 778 |
| chr10 | 81743140 | 81743211 | 72 | chr11 | 3568531 | 3569439 | 909 |
| chr10 | 82295660 | 82295909 | 250 | chr11 | 4339421 | 4339991 | 571 |
| chr10 | 85997015 | 85997255 | 241 | chr11 | 4452591 | 4452991 | 401 |
| chr10 | 88123524 | 88123578 | 55 | chr11 | 5192473 | 5192964 | 492 |
| chr10 | 88295206 | 88295354 | 149 | chr11 | 5529170 | 5529598 | 429 |
| chr10 | 88702581 | 88702664 | 84 | chr11 | 6340698 | 6341081 | 384 |
| chr11 | 7870304 | 7870657 | 354 | chr11 | 68517870 | 68518155 | 286 |
| chr11 | 14926692 | 14927370 | 679 | chr11 | 68610416 | 68611297 | 882 |
| chr11 | 16632509 | 16632602 | 94 | chr11 | 68672413 | 68672544 | 132 |
| chr11 | 19024556 | 19025005 | 450 | chr11 | 69326560 | 69326647 | 88 |
| chr11 | 20153433 | 20153484 | 52 | chr11 | 70456722 | 70456835 | 114 |
| chr11 | 20229894 | 20230564 | 671 | chr11 | 71049983 | 71050396 | 414 |
| chr11 | 20627225 | 20627255 | 31 | chr11 | 71790208 | 71790326 | 119 |
| chr11 | 22362561 | 22363207 | 647 | chr11 | 72301235 | 72301725 | 491 |
| chr11 | 22513316 | 22513710 | 395 | chr11 | 72498572 | 72498706 | 135 |
| chr11 | 31840908 | 31841940 | 1033 | chr11 | 73357140 | 73357314 | 175 |
| chr11 | 31845108 | 31845253 | 146 | chr11 | 73499621 | 73499809 | 189 |
| chr11 | 36477229 | 36477458 | 230 | chr11 | 76033041 | 76033289 | 249 |
| chr11 | 43895405 | 43895502 | 98 | chr11 | 76348671 | 76348765 | 95 |
| chr11 | 43896979 | 43897779 | 801 | chr11 | 76849389 | 76849463 | 75 |
| chr11 | 44324879 | 44325408 | 530 | chr11 | 76858767 | 76859015 | 249 |
| chr11 | 45671831 | 45671876 | 46 | chr11 | 77734036 | 77734281 | 246 |
| chr11 | 48412827 | 48413146 | 320 | chr11 | 85396372 | 85396816 | 445 |
| chr11 | 48451249 | 48451882 | 634 | chr11 | 91956571 | 91956797 | 227 |
| chr11 | 48555142 | 48555262 | 121 | chr11 | 94278394 | 94278737 | 344 |
| chr11 | 48568894 | 48569310 | 417 | chr11 | 94770623 | 94771091 | 469 |
| chr11 | 48624841 | 48625442 | 602 | chr11 | 94883425 | 94883897 | 473 |
| chr11 | 49342563 | 49342801 | 239 | chr11 | 107337020 | 107337389 | 370 |
| chr11 | 50158738 | 50158979 | 242 | chr11 | 113258455 | 113258734 | 280 |
| chr11 | 56280231 | 56280537 | 307 | chr11 | 115530932 | 115531263 | 332 |
| chr11 | 57601395 | 57601770 | 376 | chr11 | 115587563 | 115587718 | 156 |
| chr11 | 57639402 | 57640780 | 1379 | chr11 | 116228863 | 116229003 | 141 |
| chr11 | 57647103 | 57647652 | 550 | chr11 | 118221181 | 118221387 | 207 |
| chr11 | 57698126 | 57698467 | 342 | chr11 | 118480534 | 118481521 | 988 |
| chr11 | 57899888 | 57901265 | 1378 | chr11 | 118780453 | 118780610 | 158 |
| chr11 | 57933149 | 57934755 | 1607 | chr11 | 118842425 | 118842563 | 139 |
| chr11 | 57955437 | 57956496 | 1060 | chr11 | 123066875 | 123066937 | 63 |
| chr11 | 57958326 | 57958438 | 113 | chr11 | 126174593 | 126174722 | 130 |
| chr11 | 57985199 | 57986139 | 941 | chr11 | 128800041 | 128800165 | 125 |
| chr11 | 58079021 | 58080086 | 1066 | chr11 | 131056727 | 131056943 | 217 |
| chr11 | 59489354 | 59489515 | 162 | chr11 | 131526607 | 131526851 | 245 |
| chr11 | 59627629 | 59627849 | 221 | chr11 | 134315524 | 134315827 | 304 |
| chr11 | 59833005 | 59833958 | 954 | chr12 | 752770 | 752850 | 81 |
| chr11 | 60620190 | 60620294 | 105 | chr12 | 1771967 | 1772006 | 40 |
| chr11 | 60718442 | 60718578 | 137 | chr12 | 1906206 | 1906632 | 427 |
| chr11 | 60775644 | 60776119 | 476 | chr12 | 1973809 | 1974246 | 438 |
| chr11 | 61671963 | 61672078 | 116 | chr12 | 2033659 | 2033850 | 192 |
| chr11 | 61723296 | 61723323 | 28 | chr12 | 2339393 | 2339561 | 169 |
| chr11 | 62693566 | 62693622 | 57 | chr12 | 2800287 | 2800313 | 27 |
| chr11 | 64443046 | 64443129 | 84 | chr12 | 3948219 | 3948414 | 196 |
| chr11 | 64479516 | 64479580 | 65 | chr12 | 4484358 | 4484417 | 60 |
| chr11 | 64814948 | 64815698 | 751 | chr12 | 4489659 | 4491243 | 1585 |
| chr11 | 65575446 | 65577394 | 1949 | chr12 | 4511746 | 4512200 | 455 |
| chr11 | 65579136 | 65579573 | 438 | chr12 | 4557672 | 4558159 | 488 |
| chr11 | 66207826 | 66211095 | 3270 | chr12 | 4923842 | 4923932 | 91 |
| chr12 | 5041566 | 5042102 | 537 | chr12 | 34781194 | 34781813 | 620 |
| chr12 | 5049049 | 5050696 | 1648 | chr12 | 37975601 | 37975904 | 304 |
| chr12 | 5106896 | 5107459 | 564 | chr12 | 38540879 | 38541132 | 254 |
| chr12 | 5949186 | 5949746 | 561 | chr12 | 39538708 | 39539049 | 342 |
| chr12 | 5970433 | 5970852 | 420 | chr12 | 48506507 | 48507094 | 588 |
| chr12 | 6165898 | 6166078 | 181 | chr12 | 49484921 | 49485137 | 217 |
| chr12 | 6729468 | 6730038 | 571 | chr12 | 49689977 | 49690069 | 93 |
| chr12 | 6883077 | 6883233 | 157 | chr12 | 49726735 | 49728619 | 1885 |
| chr12 | 7662200 | 7662694 | 495 | chr12 | 49729685 | 49729971 | 287 |
| chr12 | 8523673 | 8524336 | 664 | chr12 | 49938044 | 49938089 | 46 |
| chr12 | 9993278 | 9993529 | 252 | chr12 | 49942968 | 49943272 | 305 |
| chr12 | 10339004 | 10339143 | 140 | chr12 | 50349130 | 50349343 | 214 |
| chr12 | 10786797 | 10787577 | 781 | chr12 | 51236851 | 51236908 | 58 |
| chr12 | 12225207 | 12225286 | 80 | chr12 | 51786132 | 51786560 | 429 |
| chr12 | 20099032 | 20100079 | 1048 | chr12 | 52281735 | 52281784 | 50 |
| chr12 | 22441349 | 22441561 | 213 | chr12 | 52482402 | 52482525 | 124 |
| chr12 | 26844579 | 26844839 | 261 | chr12 | 52575700 | 52575863 | 164 |
| chr12 | 28124196 | 28124472 | 277 | chr12 | 52626619 | 52627481 | 863 |
| chr12 | 28127380 | 28127847 | 468 | chr12 | 52644094 | 52644121 | 28 |
| chr12 | 30322764 | 30323555 | 792 | chr12 | 52685060 | 52685241 | 182 |
| chr12 | 30354268 | 30354612 | 345 | chr12 | 53125477 | 53125928 | 452 |
| chr12 | 30975766 | 30976292 | 527 | chr12 | 53612519 | 53612659 | 141 |
| chr12 | 32912601 | 32912750 | 150 | chr12 | 53765641 | 53765772 | 132 |
| chr12 | 33970597 | 33971742 | 1146 | chr12 | 53895889 | 53896166 | 278 |
| chr12 | 34001547 | 34001837 | 291 | chr12 | 54204196 | 54205392 | 1197 |
| chr12 | 34150380 | 34150858 | 479 | chr12 | 54396706 | 54396966 | 261 |
| chr12 | 34218166 | 34218625 | 460 | chr12 | 54420652 | 54421002 | 351 |
| chr12 | 34323564 | 34323739 | 176 | chr12 | 54424203 | 54425525 | 1323 |
| chr12 | 34461467 | 34461738 | 272 | chr12 | 54428303 | 54428505 | 203 |
| chr12 | 34466621 | 34466852 | 232 | chr12 | 54440604 | 54441882 | 1279 |
| chr12 | 34473553 | 34474473 | 921 | chr12 | 54454163 | 54454435 | 273 |
| chr12 | 34478998 | 34479224 | 227 | chr12 | 54475125 | 54475792 | 668 |
| chr12 | 34486236 | 34487909 | 1674 | chr12 | 55030716 | 55031267 | 552 |
| chr12 | 34488977 | 34491048 | 2072 | chr12 | 55037894 | 55041645 | 3752 |
| chr12 | 34494743 | 34495429 | 687 | chr12 | 55179317 | 55180145 | 829 |
| chr12 | 34498710 | 34499732 | 1023 | chr12 | 55196884 | 55197075 | 192 |
| chr12 | 34501375 | 34501704 | 330 | chr12 | 55259962 | 55260821 | 860 |
| chr12 | 34508773 | 34509987 | 1215 | chr12 | 55282498 | 55282592 | 95 |
| chr12 | 34514054 | 34514319 | 266 | chr12 | 57082875 | 57082970 | 96 |
| chr12 | 34515633 | 34515935 | 303 | chr12 | 57618547 | 57619684 | 1138 |
| chr12 | 34517234 | 34519014 | 1781 | chr12 | 58013206 | 58013545 | 340 |
| chr12 | 34520528 | 34523413 | 2886 | chr12 | 58021690 | 58022502 | 813 |
| chr12 | 34524707 | 34528016 | 3310 | chr12 | 58049391 | 58049507 | 117 |
| chr12 | 34538391 | 34538699 | 309 | chr12 | 63525183 | 63525456 | 274 |
| chr12 | 34539988 | 34540119 | 132 | chr12 | 66564210 | 66564265 | 56 |
| chr12 | 34546282 | 34548303 | 2022 | chr12 | 75601176 | 75601207 | 32 |
| chr12 | 34555660 | 34556336 | 677 | chr12 | 77259898 | 77260243 | 346 |
| chr12 | 34749482 | 34749869 | 388 | chr12 | 80906873 | 80907245 | 373 |
| chr12 | 34757536 | 34757786 | 251 | chr12 | 81102356 | 81102694 | 339 |
| chr12 | 81110594 | 81110933 | 340 | chr12 | 132955671 | 132956890 | 1220 |
| chr12 | 85672886 | 85672910 | 25 | chr12 | 132996466 | 132996609 | 144 |
| chr12 | 101110151 | 101110515 | 365 | chr12 | 133008035 | 133008179 | 145 |
| chr12 | 103344504 | 103345473 | 970 | chr13 | 19318464 | 19320233 | 1770 |
| chr12 | 103353252 | 103353458 | 207 | chr13 | 19746263 | 19746597 | 335 |
| chr12 | 103360208 | 103360557 | 350 | chr13 | 19986426 | 19986959 | 534 |
| chr12 | 109487105 | 109487464 | 360 | chr13 | 20832619 | 20832782 | 164 |
| chr12 | 111282806 | 111282937 | 132 | chr13 | 22386503 | 22386653 | 151 |
| chr12 | 111403980 | 111404191 | 212 | chr13 | 23500331 | 23501344 | 1014 |
| chr12 | 111610159 | 111610559 | 401 | chr13 | 24269853 | 24269999 | 147 |
| chr12 | 111647566 | 111647747 | 182 | chr13 | 26551404 | 26551587 | 184 |
| chr12 | 111713247 | 111713311 | 65 | chr13 | 28382835 | 28383516 | 682 |
| chr12 | 113515631 | 113515735 | 105 | chr13 | 28388060 | 28389833 | 1774 |
| chr12 | 113835004 | 113836374 | 1371 | chr13 | 28424940 | 28426547 | 1608 |
| chr12 | 113895942 | 113896525 | 584 | chr13 | 29532220 | 29532502 | 283 |
| chr12 | 113928452 | 113929090 | 639 | chr13 | 29844779 | 29844883 | 105 |
| chr12 | 114416881 | 114417382 | 502 | chr13 | 32984212 | 32984317 | 106 |
| chr12 | 114472706 | 114474966 | 2261 | chr13 | 33591301 | 33591418 | 118 |
| chr12 | 114568440 | 114569302 | 863 | chr13 | 36045439 | 36045614 | 176 |
| chr12 | 114926970 | 114927219 | 250 | chr13 | 36052717 | 36053119 | 403 |
| chr12 | 114951323 | 114951740 | 418 | chr13 | 40746370 | 40746627 | 258 |
| chr12 | 114962019 | 114962313 | 295 | chr13 | 50701322 | 50702015 | 694 |
| chr12 | 115107268 | 115107381 | 114 | chr13 | 50706306 | 50707696 | 1391 |
| chr12 | 115130020 | 115130082 | 63 | chr13 | 51417396 | 51417832 | 437 |
| chr12 | 115173396 | 115174184 | 789 | chr13 | 53774705 | 53774908 | 204 |
| chr12 | 116043992 | 116044036 | 45 | chr13 | 58207445 | 58208079 | 635 |
| chr12 | 117563097 | 117563464 | 368 | chr13 | 91827442 | 91827714 | 273 |
| chr12 | 119165225 | 119165925 | 701 | chr13 | 95354253 | 95354411 | 159 |
| chr12 | 119591394 | 119592081 | 688 | chr13 | 100578593 | 100578945 | 353 |
| chr12 | 120794714 | 120794779 | 66 | chr13 | 109792916 | 109793686 | 771 |
| chr12 | 122231013 | 122231063 | 51 | chr13 | 113945276 | 113945391 | 116 |
| chr12 | 123373037 | 123373270 | 234 | chr13 | 113981896 | 113982291 | 396 |
| chr12 | 124866460 | 124866517 | 58 | chr13 | 114523828 | 114524159 | 332 |
| chr12 | 125033820 | 125033933 | 114 | chr14 | 21184391 | 21185815 | 1425 |
| chr12 | 125765136 | 125765320 | 185 | chr14 | 21288621 | 21288992 | 372 |
| chr12 | 126345923 | 126346486 | 564 | chr14 | 21292195 | 21293292 | 1098 |
| chr12 | 126675335 | 126675432 | 98 | chr14 | 21300503 | 21300990 | 488 |
| chr12 | 127210615 | 127210920 | 306 | chr14 | 21328577 | 21328716 | 140 |
| chr12 | 127765195 | 127765339 | 145 | chr14 | 21367160 | 21368662 | 1503 |
| chr12 | 130554864 | 130555000 | 137 | chr14 | 21385944 | 21387215 | 1272 |
| chr12 | 130785450 | 130785549 | 100 | chr14 | 21388908 | 21389686 | 779 |
| chr12 | 131006919 | 131007133 | 215 | chr14 | 21418836 | 21418989 | 154 |
| chr12 | 131061197 | 131061321 | 125 | chr14 | 22080554 | 22081279 | 726 |
| chr12 | 131104206 | 131104413 | 208 | chr14 | 22085604 | 22087302 | 1699 |
| chr12 | 132620943 | 132621043 | 101 | chr14 | 22093664 | 22094625 | 962 |
| chr12 | 132652412 | 132653156 | 745 | chr14 | 22125367 | 22126031 | 665 |
| chr12 | 132703959 | 132704321 | 363 | chr14 | 22193104 | 22194089 | 986 |
| chr12 | 132820146 | 132820200 | 55 | chr14 | 22205088 | 22206624 | 1537 |
| chr12 | 132913602 | 132913700 | 99 | chr14 | 22221974 | 22222682 | 709 |
| chr14 | 22256795 | 22258540 | 1746 | chr14 | 96180717 | 96180760 | 44 |
| chr14 | 22480668 | 22482035 | 1368 | chr14 | 96410830 | 96411312 | 483 |
| chr14 | 22493684 | 22495588 | 1905 | chr14 | 97492100 | 97492286 | 187 |
| chr14 | 22496990 | 22497538 | 549 | chr14 | 100125700 | 100125865 | 166 |
| chr14 | 22615266 | 22616647 | 1382 | chr14 | 102172124 | 102172687 | 564 |
| chr14 | 29643475 | 29644225 | 751 | chr14 | 104338233 | 104338639 | 407 |
| chr14 | 29763423 | 29765727 | 2305 | chr14 | 105995472 | 105996178 | 707 |
| chr14 | 29883148 | 29884282 | 1135 | chr14 | 106066694 | 106067503 | 810 |
| chr14 | 34269188 | 34270420 | 1233 | chr14 | 106072877 | 106073429 | 553 |
| chr14 | 35805885 | 35806414 | 530 | chr14 | 106087918 | 106089505 | 1588 |
| chr14 | 35871618 | 35871854 | 237 | chr14 | 106094351 | 106096243 | 1893 |
| chr14 | 36593402 | 36593598 | 197 | chr14 | 106112791 | 106114627 | 1837 |
| chr14 | 36707733 | 36708065 | 333 | chr14 | 106130630 | 106134033 | 3404 |
| chr14 | 36845922 | 36846619 | 698 | chr14 | 106154371 | 106154468 | 98 |
| chr14 | 36861726 | 36861834 | 109 | chr14 | 106165410 | 106165608 | 199 |
| chr14 | 36937006 | 36937135 | 130 | chr14 | 106173826 | 106174806 | 981 |
| chr14 | 36946212 | 36946615 | 404 | chr14 | 106190169 | 106190396 | 228 |
| chr14 | 36955283 | 36956843 | 1561 | chr14 | 106202832 | 106202901 | 70 |
| chr14 | 46533168 | 46533259 | 92 | chr14 | 106206715 | 106206877 | 163 |
| chr14 | 51862342 | 51862495 | 154 | chr14 | 106207993 | 106209208 | 1216 |
| chr14 | 54418701 | 54418778 | 78 | chr14 | 106212273 | 106215432 | 3160 |
| chr14 | 55707748 | 55707831 | 84 | chr14 | 106216554 | 106218111 | 1558 |
| chr14 | 55907252 | 55907340 | 89 | chr14 | 106226624 | 106227842 | 1219 |
| chr14 | 57265461 | 57265500 | 40 | chr14 | 106230708 | 106235784 | 5077 |
| chr14 | 61747057 | 61747079 | 23 | chr14 | 106239167 | 106241517 | 2351 |
| chr14 | 64701761 | 64701940 | 180 | chr14 | 106282704 | 106283163 | 460 |
| chr14 | 65183484 | 65183709 | 226 | chr14 | 106306751 | 106307736 | 986 |
| chr14 | 69015084 | 69015362 | 279 | chr14 | 106331626 | 106331925 | 300 |
| chr14 | 69095026 | 69095407 | 382 | chr14 | 106345452 | 106345681 | 230 |
| chr14 | 73203762 | 73204104 | 343 | chr14 | 106369777 | 106370076 | 300 |
| chr14 | 74207791 | 74207972 | 182 | chr14 | 106398826 | 106399173 | 348 |
| chr14 | 74684842 | 74685485 | 644 | chr14 | 106434442 | 106434640 | 199 |
| chr14 | 75077666 | 75077736 | 71 | chr14 | 106438409 | 106438520 | 112 |
| chr14 | 75925947 | 75926221 | 275 | chr14 | 106660649 | 106661756 | 1108 |
| chr14 | 76102664 | 76102825 | 162 | chr14 | 106775893 | 106776299 | 407 |
| chr14 | 77940785 | 77941175 | 391 | chr14 | 107078767 | 107079849 | 1083 |
| chr14 | 78108000 | 78108190 | 191 | chr14 | 107252453 | 107252586 | 134 |
| chr14 | 78127160 | 78127497 | 338 | chr15 | 20544115 | 20544213 | 99 |
| chr14 | 89956447 | 89956565 | 119 | chr15 | 21326491 | 21326577 | 87 |
| chr14 | 90679671 | 90680240 | 570 | chr15 | 22511339 | 22512630 | 1292 |
| chr14 | 92729963 | 92730282 | 320 | chr15 | 22529217 | 22529398 | 182 |
| chr14 | 93672453 | 93672634 | 182 | chr15 | 22683242 | 22683330 | 89 |
| chr14 | 94179834 | 94179933 | 100 | chr15 | 22798802 | 22799377 | 576 |
| chr14 | 94254077 | 94254311 | 235 | chr15 | 22832532 | 22832983 | 452 |
| chr14 | 94812750 | 94813014 | 265 | chr15 | 23452539 | 23452612 | 74 |
| chr14 | 95177512 | 95177685 | 174 | chr15 | 23617194 | 23617616 | 423 |
| chr14 | 95237252 | 95238205 | 954 | chr15 | 23640638 | 23641009 | 372 |
| chr14 | 95402991 | 95403225 | 235 | chr15 | 23668338 | 23668626 | 289 |
| chr14 | 95826680 | 95826868 | 189 | chr15 | 23687462 | 23687772 | 311 |
| chr15 | 23810717 | 23812141 | 1425 | chr15 | 89951416 | 89951500 | 85 |
| chr15 | 26915257 | 26915709 | 453 | chr15 | 89953033 | 89953458 | 426 |
| chr15 | 27111476 | 27111605 | 130 | chr15 | 93652794 | 93652890 | 97 |
| chr15 | 28437122 | 28437183 | 62 | chr15 | 98236065 | 98236179 | 115 |
| chr15 | 28702823 | 28702924 | 102 | chr15 | 100742125 | 100742237 | 113 |
| chr15 | 29210743 | 29210863 | 121 | chr15 | 101084484 | 101085060 | 577 |
| chr15 | 29395841 | 29396147 | 307 | chr15 | 102193467 | 102193559 | 93 |
| chr15 | 30517920 | 30518136 | 217 | chr16 | 875106 | 876238 | 1133 |
| chr15 | 31775371 | 31776400 | 1030 | chr16 | 1027331 | 1027598 | 268 |
| chr15 | 32604873 | 32605409 | 537 | chr16 | 1033580 | 1034255 | 676 |
| chr15 | 33154165 | 33154256 | 92 | chr16 | 1035460 | 1037982 | 2523 |
| chr15 | 41787977 | 41788286 | 310 | chr16 | 1052613 | 1052757 | 145 |
| chr15 | 42174199 | 42174579 | 381 | chr16 | 1069306 | 1069761 | 456 |
| chr15 | 45314866 | 45314929 | 64 | chr16 | 1076698 | 1077664 | 967 |
| chr15 | 45408790 | 45408826 | 37 | chr16 | 1092992 | 1096344 | 3353 |
| chr15 | 45427351 | 45427382 | 32 | chr16 | 1118570 | 1119026 | 457 |
| chr15 | 45996472 | 45996836 | 365 | chr16 | 1128269 | 1129001 | 733 |
| chr15 | 53976721 | 53976836 | 116 | chr16 | 1297447 | 1297715 | 269 |
| chr15 | 54773780 | 54774590 | 811 | chr16 | 1312274 | 1312872 | 599 |
| chr15 | 55000594 | 55000990 | 397 | chr16 | 1941885 | 1942747 | 863 |
| chr15 | 55047814 | 55048773 | 960 | chr16 | 2029419 | 2029472 | 54 |
| chr15 | 55304762 | 55305350 | 589 | chr16 | 2141398 | 2141443 | 46 |
| chr15 | 55308619 | 55309359 | 741 | chr16 | 2287797 | 2287882 | 86 |
| chr15 | 55458346 | 55458449 | 104 | chr16 | 2532909 | 2533396 | 488 |
| chr15 | 59157135 | 59157417 | 283 | chr16 | 2841218 | 2841594 | 377 |
| chr15 | 63245330 | 63245530 | 201 | chr16 | 2855817 | 2856979 | 1163 |
| chr15 | 63753681 | 63753711 | 31 | chr16 | 3130144 | 3130255 | 112 |
| chr15 | 65186262 | 65186662 | 401 | chr16 | 3142376 | 3143343 | 968 |
| chr15 | 66364205 | 66364333 | 129 | chr16 | 3254252 | 3254508 | 257 |
| chr15 | 66546859 | 66547493 | 635 | chr16 | 3306855 | 3307393 | 539 |
| chr15 | 66939853 | 66939993 | 141 | chr16 | 3345186 | 3345785 | 600 |
| chr15 | 68549178 | 68549298 | 121 | chr16 | 3550141 | 3550402 | 262 |
| chr15 | 68943564 | 68943635 | 72 | chr16 | 4715128 | 4715473 | 346 |
| chr15 | 69432690 | 69432997 | 308 | chr16 | 5041752 | 5041955 | 204 |
| chr15 | 70354463 | 70354999 | 537 | chr16 | 5052407 | 5053919 | 1513 |
| chr15 | 70744668 | 70744804 | 137 | chr16 | 5058450 | 5058606 | 157 |
| chr15 | 71507427 | 71507621 | 195 | chr16 | 5067748 | 5068561 | 814 |
| chr15 | 72520632 | 72520788 | 157 | chr16 | 5070626 | 5070756 | 131 |
| chr15 | 72979616 | 72979774 | 159 | chr16 | 5511311 | 5511678 | 368 |
| chr15 | 74425185 | 74425602 | 418 | chr16 | 11327053 | 11327285 | 233 |
| chr15 | 76484262 | 76484319 | 58 | chr16 | 14493896 | 14494070 | 175 |
| chr15 | 77320557 | 77320956 | 400 | chr16 | 19244110 | 19244589 | 480 |
| chr15 | 79123216 | 79123576 | 361 | chr16 | 19394470 | 19394689 | 220 |
| chr15 | 79376693 | 79376922 | 230 | chr16 | 20347952 | 20349234 | 1283 |
| chr15 | 79725483 | 79725750 | 268 | chr16 | 20379780 | 20380862 | 1083 |
| chr15 | 79854831 | 79854928 | 98 | chr16 | 21838930 | 21839457 | 528 |
| chr15 | 83518294 | 83518445 | 152 | chr16 | 22137566 | 22137696 | 131 |
| chr15 | 89239304 | 89239376 | 73 | chr16 | 27063250 | 27063556 | 307 |
| chr15 | 89920794 | 89922704 | 1911 | chr16 | 27797654 | 27797764 | 111 |
| chr16 | 29031870 | 29032012 | 143 | chr16 | 81679029 | 81679185 | 157 |
| chr16 | 29086428 | 29086494 | 67 | chr16 | 82044811 | 82045283 | 473 |
| chr16 | 31362687 | 31362975 | 289 | chr16 | 85320686 | 85320752 | 67 |
| chr16 | 31383363 | 31383568 | 206 | chr16 | 86705764 | 86705948 | 185 |
| chr16 | 31384675 | 31384820 | 146 | chr16 | 87194583 | 87194772 | 190 |
| chr16 | 31425870 | 31426809 | 940 | chr16 | 87638291 | 87638669 | 379 |
| chr16 | 31567988 | 31568597 | 610 | chr16 | 89041965 | 89042186 | 222 |
| chr16 | 31651383 | 31652053 | 671 | chr16 | 89055677 | 89056357 | 681 |
| chr16 | 32033587 | 32034074 | 488 | chr16 | 89072182 | 89072814 | 633 |
| chr16 | 33407038 | 33407528 | 491 | chr16 | 89078132 | 89080740 | 2609 |
| chr16 | 34212002 | 34212361 | 360 | chr16 | 89084537 | 89085330 | 794 |
| chr16 | 34229704 | 34229806 | 103 | chr16 | 89089164 | 89090572 | 1409 |
| chr16 | 34521926 | 34522055 | 130 | chr16 | 89099948 | 89100337 | 390 |
| chr16 | 46878390 | 46878462 | 73 | chr16 | 89102982 | 89103105 | 124 |
| chr16 | 47892451 | 47892554 | 104 | chr16 | 89105925 | 89106423 | 499 |
| chr16 | 48010663 | 48010747 | 85 | chr16 | 89107703 | 89107835 | 133 |
| chr16 | 48845353 | 48845527 | 175 | chr16 | 89111065 | 89111126 | 62 |
| chr16 | 49311803 | 49312408 | 606 | chr16 | 89118637 | 89119204 | 568 |
| chr16 | 49508928 | 49509032 | 105 | chr16 | 89122737 | 89122986 | 250 |
| chr16 | 49872424 | 49872891 | 468 | chr16 | 89642754 | 89643184 | 431 |
| chr16 | 50707547 | 50707799 | 253 | chr17 | 259426 | 259570 | 145 |
| chr16 | 50875108 | 50875363 | 256 | chr17 | 1184147 | 1184254 | 108 |
| chr16 | 54210430 | 54210538 | 109 | chr17 | 1960236 | 1962552 | 2317 |
| chr16 | 54315585 | 54316157 | 573 | chr17 | 2827559 | 2827646 | 88 |
| chr16 | 54322358 | 54325841 | 3484 | chr17 | 3028803 | 3030414 | 1612 |
| chr16 | 54685651 | 54685978 | 328 | chr17 | 3034963 | 3035841 | 879 |
| chr16 | 54967714 | 54969431 | 1718 | chr17 | 3049286 | 3050256 | 971 |
| chr16 | 54971587 | 54974166 | 2580 | chr17 | 3119049 | 3119330 | 282 |
| chr16 | 55360333 | 55360401 | 69 | chr17 | 3165145 | 3165568 | 424 |
| chr16 | 55361525 | 55361686 | 162 | chr17 | 3171784 | 3171996 | 213 |
| chr16 | 55362754 | 55363378 | 625 | chr17 | 3194697 | 3195428 | 732 |
| chr16 | 55364661 | 55365943 | 1283 | chr17 | 3212988 | 3214538 | 1551 |
| chr16 | 56136445 | 56136614 | 170 | chr17 | 3280846 | 3284665 | 3820 |
| chr16 | 58018409 | 58018510 | 102 | chr17 | 4802571 | 4805169 | 2599 |
| chr16 | 62442872 | 62442955 | 84 | chr17 | 5568568 | 5568683 | 116 |
| chr16 | 65093431 | 65093518 | 88 | chr17 | 5805831 | 5806377 | 547 |
| chr16 | 67183645 | 67183976 | 332 | chr17 | 5930705 | 5931113 | 409 |
| chr16 | 67199263 | 67199877 | 615 | chr17 | 6899212 | 6899326 | 115 |
| chr16 | 67233494 | 67233828 | 335 | chr17 | 7246705 | 7246859 | 155 |
| chr16 | 68113688 | 68114092 | 405 | chr17 | 7907116 | 7907149 | 34 |
| chr16 | 68269149 | 68269856 | 708 | chr17 | 8296167 | 8296315 | 149 |
| chr16 | 70321957 | 70322037 | 81 | chr17 | 8601985 | 8602180 | 196 |
| chr16 | 70905156 | 70905984 | 829 | chr17 | 11024744 | 11025107 | 364 |
| chr16 | 71407033 | 71407229 | 197 | chr17 | 11351254 | 11351341 | 88 |
| chr16 | 71580292 | 71580593 | 302 | chr17 | 11369149 | 11369304 | 156 |
| chr16 | 72961557 | 72961650 | 94 | chr17 | 12127727 | 12128512 | 786 |
| chr16 | 73266649 | 73267121 | 473 | chr17 | 12989524 | 12989712 | 189 |
| chr16 | 79451567 | 79451748 | 182 | chr17 | 15652887 | 15653052 | 166 |
| chr16 | 81616834 | 81616923 | 90 | chr17 | 15686241 | 15686310 | 70 |
| chr17 | 18061494 | 18061879 | 386 | chr17 | 71897983 | 71898065 | 83 |
| chr17 | 19627882 | 19628209 | 328 | chr17 | 72462623 | 72462737 | 115 |
| chr17 | 19883432 | 19883550 | 119 | chr17 | 73713184 | 73713643 | 460 |
| chr17 | 20687616 | 20687633 | 18 | chr17 | 74268070 | 74268300 | 231 |
| chr17 | 22016920 | 22017005 | 86 | chr17 | 75143112 | 75143220 | 109 |
| chr17 | 26634075 | 26634270 | 196 | chr17 | 75278133 | 75278170 | 38 |
| chr17 | 26711390 | 26712303 | 914 | chr17 | 76037095 | 76037304 | 210 |
| chr17 | 27940013 | 27940122 | 110 | chr17 | 76123142 | 76123525 | 384 |
| chr17 | 29335980 | 29336372 | 393 | chr17 | 76170680 | 76170820 | 141 |
| chr17 | 30023233 | 30023521 | 289 | chr17 | 77351267 | 77351390 | 124 |
| chr17 | 30243999 | 30244330 | 332 | chr17 | 77644645 | 77644664 | 20 |
| chr17 | 32964615 | 32964925 | 311 | chr17 | 77788385 | 77789793 | 1409 |
| chr17 | 34818997 | 34819245 | 249 | chr18 | 10032401 | 10032984 | 584 |
| chr17 | 36009793 | 36010175 | 383 | chr18 | 11947710 | 11947853 | 144 |
| chr17 | 36684600 | 36684936 | 337 | chr18 | 12911817 | 12912258 | 442 |
| chr17 | 36997449 | 36997527 | 79 | chr18 | 13500430 | 13500568 | 139 |
| chr17 | 37768930 | 37769019 | 90 | chr18 | 13610930 | 13611185 | 256 |
| chr17 | 38855747 | 38856010 | 264 | chr18 | 13641735 | 13642202 | 468 |
| chr17 | 39323854 | 39324304 | 451 | chr18 | 24131285 | 24131498 | 214 |
| chr17 | 40191977 | 40192000 | 24 | chr18 | 34833574 | 34833617 | 44 |
| chr17 | 40274620 | 40274881 | 262 | chr18 | 34854359 | 34854986 | 628 |
| chr17 | 40440991 | 40441496 | 506 | chr18 | 42596470 | 42596995 | 526 |
| chr17 | 41496314 | 41496488 | 175 | chr18 | 44256237 | 44256338 | 102 |
| chr17 | 42052336 | 42052646 | 311 | chr18 | 44703033 | 44703131 | 99 |
| chr17 | 42246292 | 42246353 | 62 | chr18 | 46102017 | 46102203 | 187 |
| chr17 | 43832416 | 43832669 | 254 | chr18 | 47794615 | 47794986 | 372 |
| chr17 | 43938592 | 43939012 | 421 | chr18 | 56514733 | 56515047 | 315 |
| chr17 | 44847253 | 44847309 | 57 | chr18 | 56941929 | 56942021 | 93 |
| chr17 | 44896931 | 44897606 | 676 | chr18 | 61669881 | 61670110 | 230 |
| chr17 | 44961444 | 44961613 | 170 | chr18 | 71442187 | 71442401 | 215 |
| chr17 | 47234251 | 47234786 | 536 | chr18 | 74064256 | 74064323 | 68 |
| chr17 | 47245333 | 47246448 | 1116 | chr18 | 74396138 | 74396443 | 306 |
| chr17 | 48858811 | 48858877 | 67 | chr18 | 75962942 | 75963212 | 271 |
| chr17 | 53341014 | 53341102 | 89 | chr18 | 76240803 | 76240844 | 42 |
| chr17 | 54696809 | 54696912 | 104 | chr18 | 76266026 | 76266359 | 334 |
| chr17 | 56276469 | 56276803 | 335 | chr18 | 76322346 | 76322412 | 67 |
| chr17 | 56565000 | 56565492 | 493 | chr18 | 76417526 | 76417771 | 246 |
| chr17 | 58179983 | 58180069 | 87 | chr18 | 76494215 | 76494459 | 245 |
| chr17 | 59473326 | 59473748 | 423 | chr18 | 76681368 | 76681441 | 74 |
| chr17 | 59481234 | 59483164 | 1931 | chr18 | 76774006 | 76774241 | 236 |
| chr17 | 59485522 | 59485538 | 17 | chr18 | 76790593 | 76791293 | 701 |
| chr17 | 59528892 | 59530252 | 1361 | chr18 | 76828652 | 76828709 | 58 |
| chr17 | 59531471 | 59535761 | 4291 | chr18 | 77359685 | 77359791 | 107 |
| chr17 | 62141400 | 62141508 | 109 | chr18 | 77531686 | 77532180 | 495 |
| chr17 | 65487278 | 65488097 | 820 | chr18 | 77545028 | 77545166 | 139 |
| chr17 | 70112035 | 70112906 | 872 | chr18 | 77630540 | 77630843 | 304 |
| chr17 | 70472839 | 70473039 | 201 | chr19 | 382370 | 382519 | 150 |
| chr17 | 70636580 | 70636838 | 259 | chr19 | 427297 | 427395 | 99 |
| chr17 | 71715942 | 71716007 | 66 | chr19 | 468478 | 468723 | 246 |
| chr19 | 855624 | 856301 | 678 | chr19 | 6921878 | 6922402 | 525 |
| chr19 | 1041756 | 1041861 | 106 | chr19 | 6929994 | 6930752 | 759 |
| chr19 | 1466938 | 1467009 | 72 | chr19 | 6986285 | 6986565 | 281 |
| chr19 | 1525557 | 1526827 | 1271 | chr19 | 7000175 | 7001140 | 966 |
| chr19 | 1529513 | 1530087 | 575 | chr19 | 7433708 | 7433982 | 275 |
| chr19 | 1533802 | 1533843 | 42 | chr19 | 7643954 | 7644900 | 947 |
| chr19 | 1683776 | 1685049 | 1274 | chr19 | 7707408 | 7707532 | 125 |
| chr19 | 1690142 | 1690192 | 51 | chr19 | 7711279 | 7711417 | 139 |
| chr19 | 1742350 | 1743570 | 1221 | chr19 | 7713356 | 7713565 | 210 |
| chr19 | 2250804 | 2252385 | 1582 | chr19 | 7727653 | 7728800 | 1148 |
| chr19 | 2523702 | 2524093 | 392 | chr19 | 7736671 | 7738678 | 2008 |
| chr19 | 2551038 | 2551168 | 131 | chr19 | 7742902 | 7743620 | 719 |
| chr19 | 2603253 | 2603475 | 223 | chr19 | 7757059 | 7763181 | 6123 |
| chr19 | 2658094 | 2658481 | 388 | chr19 | 7767229 | 7769161 | 1933 |
| chr19 | 2674725 | 2676175 | 1451 | chr19 | 7774807 | 7775281 | 475 |
| chr19 | 2727598 | 2727791 | 194 | chr19 | 7780326 | 7780583 | 258 |
| chr19 | 2867322 | 2867555 | 234 | chr19 | 7782370 | 7782922 | 553 |
| chr19 | 3098673 | 3098794 | 122 | chr19 | 7786625 | 7786800 | 176 |
| chr19 | 3149781 | 3150232 | 452 | chr19 | 7789248 | 7793785 | 4538 |
| chr19 | 3312514 | 3312880 | 367 | chr19 | 7796513 | 7797356 | 844 |
| chr19 | 3319851 | 3321239 | 1389 | chr19 | 7809903 | 7810560 | 658 |
| chr19 | 3687816 | 3688156 | 341 | chr19 | 7814351 | 7815656 | 1306 |
| chr19 | 3736267 | 3738429 | 2163 | chr19 | 7817653 | 7818639 | 987 |
| chr19 | 3747767 | 3747983 | 217 | chr19 | 7858109 | 7858490 | 382 |
| chr19 | 3778257 | 3778310 | 54 | chr19 | 7859878 | 7860066 | 189 |
| chr19 | 3838885 | 3838956 | 72 | chr19 | 7863111 | 7863481 | 371 |
| chr19 | 4154758 | 4154986 | 229 | chr19 | 7870420 | 7870824 | 405 |
| chr19 | 4159240 | 4159809 | 570 | chr19 | 7878890 | 7880858 | 1969 |
| chr19 | 4212065 | 4212896 | 832 | chr19 | 7884925 | 7885380 | 456 |
| chr19 | 4214289 | 4216161 | 1873 | chr19 | 7888954 | 7891083 | 2130 |
| chr19 | 4236853 | 4237029 | 177 | chr19 | 7927029 | 7928018 | 990 |
| chr19 | 5742510 | 5743097 | 588 | chr19 | 8638266 | 8639032 | 767 |
| chr19 | 5758670 | 5758991 | 322 | chr19 | 8644608 | 8644713 | 106 |
| chr19 | 6637692 | 6638806 | 1115 | chr19 | 8759018 | 8759983 | 966 |
| chr19 | 6642716 | 6642929 | 214 | chr19 | 8778998 | 8780690 | 1693 |
| chr19 | 6783828 | 6784031 | 204 | chr19 | 8798246 | 8798496 | 251 |
| chr19 | 6785294 | 6786210 | 917 | chr19 | 8803129 | 8803795 | 667 |
| chr19 | 6788278 | 6788636 | 359 | chr19 | 8805942 | 8806337 | 396 |
| chr19 | 6809387 | 6810041 | 655 | chr19 | 8832097 | 8835034 | 2938 |
| chr19 | 6811266 | 6812564 | 1299 | chr19 | 8837585 | 8838000 | 416 |
| chr19 | 6822328 | 6823326 | 999 | chr19 | 8860605 | 8860843 | 239 |
| chr19 | 6826534 | 6827800 | 1267 | chr19 | 8890200 | 8890519 | 320 |
| chr19 | 6830513 | 6833509 | 2997 | chr19 | 8899326 | 8899887 | 562 |
| chr19 | 6836548 | 6838294 | 1747 | chr19 | 8998595 | 9000070 | 1476 |
| chr19 | 6849338 | 6849865 | 528 | chr19 | 9024586 | 9025176 | 591 |
| chr19 | 6854133 | 6857302 | 3170 | chr19 | 9028976 | 9030311 | 1336 |
| chr19 | 6882831 | 6883157 | 327 | chr19 | 9049654 | 9050351 | 698 |
| chr19 | 6884271 | 6884566 | 296 | chr19 | 9064150 | 9065043 | 894 |
| chr19 | 6885840 | 6886421 | 582 | chr19 | 9067438 | 9070823 | 3386 |
| chr19 | 9095797 | 9097223 | 1427 | chr19 | 13545055 | 13546891 | 1837 |
| chr19 | 9116847 | 9117337 | 491 | chr19 | 13548325 | 13549172 | 848 |
| chr19 | 9118397 | 9122752 | 4356 | chr19 | 13563259 | 13564687 | 1429 |
| chr19 | 9129061 | 9129117 | 57 | chr19 | 13571924 | 13572700 | 777 |
| chr19 | 9134956 | 9135128 | 173 | chr19 | 13577434 | 13579472 | 2039 |
| chr19 | 9137227 | 9137343 | 117 | chr19 | 13580720 | 13584242 | 3523 |
| chr19 | 9144911 | 9146389 | 1479 | chr19 | 13589515 | 13592017 | 2503 |
| chr19 | 9152506 | 9153732 | 1227 | chr19 | 13597734 | 13598231 | 498 |
| chr19 | 9160899 | 9161440 | 542 | chr19 | 13599950 | 13601286 | 1337 |
| chr19 | 9163417 | 9163472 | 56 | chr19 | 13609028 | 13612179 | 3152 |
| chr19 | 9192736 | 9193322 | 587 | chr19 | 13635860 | 13636317 | 458 |
| chr19 | 9197874 | 9198859 | 986 | chr19 | 13667489 | 13667656 | 168 |
| chr19 | 9202997 | 9203344 | 348 | chr19 | 13674048 | 13675327 | 1280 |
| chr19 | 9205028 | 9206009 | 982 | chr19 | 13678719 | 13678913 | 195 |
| chr19 | 9208118 | 9208428 | 311 | chr19 | 13681162 | 13681380 | 219 |
| chr19 | 9213844 | 9214425 | 582 | chr19 | 13766857 | 13767041 | 185 |
| chr19 | 9217651 | 9218290 | 640 | chr19 | 13785327 | 13785494 | 168 |
| chr19 | 9219377 | 9221200 | 1824 | chr19 | 14583958 | 14584177 | 220 |
| chr19 | 9227810 | 9229433 | 1624 | chr19 | 14697030 | 14697218 | 189 |
| chr19 | 9239648 | 9239746 | 99 | chr19 | 14726904 | 14727104 | 201 |
| chr19 | 9296718 | 9296995 | 278 | chr19 | 14737205 | 14738679 | 1475 |
| chr19 | 9301677 | 9302765 | 1089 | chr19 | 14747308 | 14747767 | 460 |
| chr19 | 9342816 | 9343075 | 260 | chr19 | 14750361 | 14751618 | 1258 |
| chr19 | 9381329 | 9381860 | 532 | chr19 | 14773523 | 14773976 | 454 |
| chr19 | 9703188 | 9703804 | 617 | chr19 | 14792077 | 14792351 | 275 |
| chr19 | 9838293 | 9838964 | 672 | chr19 | 14872261 | 14872624 | 364 |
| chr19 | 10157126 | 10159606 | 2481 | chr19 | 15083649 | 15083780 | 132 |
| chr19 | 10687571 | 10688259 | 689 | chr19 | 15120690 | 15120774 | 85 |
| chr19 | 11471965 | 11472183 | 219 | chr19 | 15194753 | 15195440 | 688 |
| chr19 | 11689427 | 11689846 | 420 | chr19 | 15343395 | 15344175 | 781 |
| chr19 | 11784803 | 11785165 | 363 | chr19 | 15627921 | 15629180 | 1260 |
| chr19 | 12624337 | 12624365 | 29 | chr19 | 15630833 | 15630952 | 120 |
| chr19 | 12666275 | 12666455 | 181 | chr19 | 15632677 | 15636104 | 3428 |
| chr19 | 12861976 | 12864656 | 2681 | chr19 | 15637763 | 15638911 | 1149 |
| chr19 | 12984179 | 12984261 | 83 | chr19 | 15641888 | 15642640 | 753 |
| chr19 | 13337472 | 13337576 | 105 | chr19 | 15647195 | 15648938 | 1744 |
| chr19 | 13414629 | 13414781 | 153 | chr19 | 15677324 | 15677551 | 228 |
| chr19 | 13418605 | 13419249 | 645 | chr19 | 15686102 | 15686859 | 758 |
| chr19 | 13450763 | 13451208 | 446 | chr19 | 15688288 | 15688842 | 555 |
| chr19 | 13458168 | 13459599 | 1432 | chr19 | 15702427 | 15702695 | 269 |
| chr19 | 13460761 | 13467149 | 6389 | chr19 | 15725005 | 15726591 | 1587 |
| chr19 | 13469247 | 13470016 | 770 | chr19 | 15749589 | 15750058 | 470 |
| chr19 | 13472538 | 13473784 | 1247 | chr19 | 15814092 | 15814262 | 171 |
| chr19 | 13475277 | 13476024 | 748 | chr19 | 15834818 | 15837523 | 2706 |
| chr19 | 13499289 | 13499367 | 79 | chr19 | 15904761 | 15908284 | 3524 |
| chr19 | 13511790 | 13512708 | 919 | chr19 | 15917947 | 15919117 | 1171 |
| chr19 | 13519178 | 13520578 | 1401 | chr19 | 15925025 | 15926902 | 1878 |
| chr19 | 13536858 | 13537992 | 1135 | chr19 | 15931224 | 15931887 | 664 |
| chr19 | 13543860 | 13543968 | 109 | chr19 | 15940096 | 15940254 | 159 |
| chr19 | 15942270 | 15942977 | 708 | chr19 | 31845878 | 31848192 | 2315 |
| chr19 | 15960764 | 15961203 | 440 | chr19 | 33060860 | 33061086 | 227 |
| chr19 | 15969107 | 15969468 | 362 | chr19 | 34215487 | 34215977 | 491 |
| chr19 | 15976100 | 15976430 | 331 | chr19 | 34513880 | 34514302 | 423 |
| chr19 | 16051163 | 16052052 | 890 | chr19 | 35042079 | 35043471 | 1393 |
| chr19 | 16084352 | 16085128 | 777 | chr19 | 35058605 | 35059550 | 946 |
| chr19 | 16871590 | 16872325 | 736 | chr19 | 35185002 | 35185231 | 230 |
| chr19 | 16879781 | 16881849 | 2069 | chr19 | 35275413 | 35276866 | 1454 |
| chr19 | 17023936 | 17025116 | 1181 | chr19 | 35750612 | 35750754 | 143 |
| chr19 | 17028723 | 17029629 | 907 | chr19 | 35784107 | 35784777 | 671 |
| chr19 | 17032769 | 17033178 | 410 | chr19 | 35793438 | 35793524 | 87 |
| chr19 | 17075697 | 17075809 | 113 | chr19 | 35822812 | 35822956 | 145 |
| chr19 | 17087577 | 17088187 | 611 | chr19 | 35828014 | 35829133 | 1120 |
| chr19 | 17116921 | 17119363 | 2443 | chr19 | 35833524 | 35833875 | 352 |
| chr19 | 17122092 | 17122253 | 162 | chr19 | 35835631 | 35835899 | 269 |
| chr19 | 17148968 | 17150959 | 1992 | chr19 | 35899279 | 35899376 | 98 |
| chr19 | 17152289 | 17152384 | 96 | chr19 | 35909376 | 35910116 | 741 |
| chr19 | 17439065 | 17440198 | 1134 | chr19 | 35992730 | 35993804 | 1075 |
| chr19 | 17548044 | 17548406 | 363 | chr19 | 35996810 | 35997183 | 374 |
| chr19 | 17599664 | 17599903 | 240 | chr19 | 36019231 | 36019846 | 616 |
| chr19 | 17991986 | 17992969 | 984 | chr19 | 36021363 | 36021750 | 388 |
| chr19 | 18002828 | 18003032 | 205 | chr19 | 36052148 | 36053599 | 1452 |
| chr19 | 18363458 | 18363879 | 422 | chr19 | 36058035 | 36058907 | 873 |
| chr19 | 18911328 | 18911748 | 421 | chr19 | 36246441 | 36246813 | 373 |
| chr19 | 18979486 | 18981553 | 2068 | chr19 | 36309602 | 36310379 | 778 |
| chr19 | 19624970 | 19625365 | 396 | chr19 | 36325607 | 36325686 | 80 |
| chr19 | 19650837 | 19651186 | 350 | chr19 | 37266719 | 37266951 | 233 |
| chr19 | 20059073 | 20059445 | 373 | chr19 | 38850513 | 38850864 | 352 |
| chr19 | 22518912 | 22519732 | 821 | chr19 | 38876501 | 38877484 | 984 |
| chr19 | 22625986 | 22626517 | 532 | chr19 | 39504647 | 39504806 | 160 |
| chr19 | 22631042 | 22631479 | 438 | chr19 | 39783732 | 39783952 | 221 |
| chr19 | 22681163 | 22681477 | 315 | chr19 | 40067439 | 40068308 | 870 |
| chr19 | 22756704 | 22758281 | 1578 | chr19 | 40095773 | 40095966 | 194 |
| chr19 | 22774579 | 22775056 | 478 | chr19 | 40177040 | 40177869 | 830 |
| chr19 | 22789127 | 22789900 | 774 | chr19 | 40184119 | 40185162 | 1044 |
| chr19 | 22791115 | 22794348 | 3234 | chr19 | 40261651 | 40262010 | 360 |
| chr19 | 22797033 | 22797554 | 522 | chr19 | 41195330 | 41195816 | 487 |
| chr19 | 22903964 | 22904890 | 927 | chr19 | 41384588 | 41386008 | 1421 |
| chr19 | 22919029 | 22919715 | 687 | chr19 | 41410541 | 41410752 | 212 |
| chr19 | 22981023 | 22981423 | 401 | chr19 | 41467517 | 41469249 | 1733 |
| chr19 | 23081672 | 23082654 | 983 | chr19 | 41511092 | 41511720 | 629 |
| chr19 | 23211947 | 23213667 | 1721 | chr19 | 41518043 | 41518488 | 446 |
| chr19 | 24180782 | 24182287 | 1506 | chr19 | 41524460 | 41526866 | 2407 |
| chr19 | 28143222 | 28143429 | 208 | chr19 | 41534830 | 41535530 | 701 |
| chr19 | 30064732 | 30065087 | 356 | chr19 | 41541861 | 41542746 | 886 |
| chr19 | 30088695 | 30089031 | 337 | chr19 | 41549145 | 41550213 | 1069 |
| chr19 | 30555360 | 30555416 | 57 | chr19 | 41553042 | 41554639 | 1598 |
| chr19 | 30713752 | 30714062 | 311 | chr19 | 41556109 | 41557140 | 1032 |
| chr19 | 31843190 | 31844181 | 992 | chr19 | 41558201 | 41560810 | 2610 |
| chr19 | 41593170 | 41595184 | 2015 | chr19 | 49033364 | 49033483 | 120 |
| chr19 | 41599401 | 41601594 | 2194 | chr19 | 49232280 | 49232332 | 53 |
| chr19 | 41618780 | 41619375 | 596 | chr19 | 49636532 | 49636972 | 441 |
| chr19 | 41622408 | 41623084 | 677 | chr19 | 49755785 | 49756167 | 383 |
| chr19 | 41704369 | 41704708 | 340 | chr19 | 49877390 | 49877479 | 90 |
| chr19 | 41712794 | 41713376 | 583 | chr19 | 50451071 | 50451606 | 536 |
| chr19 | 42014968 | 42015372 | 405 | chr19 | 50453829 | 50453936 | 108 |
| chr19 | 42021360 | 42021520 | 161 | chr19 | 50464545 | 50466300 | 1756 |
| chr19 | 42078801 | 42079652 | 852 | chr19 | 50660716 | 50661156 | 441 |
| chr19 | 42085036 | 42085842 | 807 | chr19 | 50663357 | 50663926 | 570 |
| chr19 | 42090373 | 42091267 | 895 | chr19 | 50803960 | 50804399 | 440 |
| chr19 | 42093768 | 42094205 | 438 | chr19 | 51460868 | 51461371 | 504 |
| chr19 | 42111430 | 42112509 | 1080 | chr19 | 51469451 | 51470425 | 975 |
| chr19 | 42118779 | 42119254 | 476 | chr19 | 51512220 | 51513297 | 1078 |
| chr19 | 42128742 | 42129471 | 730 | chr19 | 51561786 | 51562217 | 432 |
| chr19 | 42131085 | 42131869 | 785 | chr19 | 51572540 | 51572845 | 306 |
| chr19 | 42134620 | 42134954 | 335 | chr19 | 51615704 | 51619068 | 3365 |
| chr19 | 42187109 | 42187445 | 337 | chr19 | 51628699 | 51630344 | 1646 |
| chr19 | 42193790 | 42194798 | 1009 | chr19 | 51639266 | 51641407 | 2142 |
| chr19 | 42198433 | 42199306 | 874 | chr19 | 51644603 | 51644958 | 356 |
| chr19 | 42200777 | 42201682 | 906 | chr19 | 51688742 | 51689607 | 866 |
| chr19 | 42204883 | 42205239 | 357 | chr19 | 51693265 | 51695476 | 2212 |
| chr19 | 42208125 | 42208600 | 476 | chr19 | 51702855 | 51705422 | 2568 |
| chr19 | 42215059 | 42215775 | 717 | chr19 | 51738479 | 51739286 | 808 |
| chr19 | 42218771 | 42227551 | 8781 | chr19 | 51804329 | 51806141 | 1813 |
| chr19 | 42241136 | 42242085 | 950 | chr19 | 51933320 | 51934023 | 704 |
| chr19 | 42246084 | 42246162 | 79 | chr19 | 51946189 | 51947280 | 1092 |
| chr19 | 42251314 | 42252608 | 1295 | chr19 | 51955036 | 51956263 | 1228 |
| chr19 | 42262763 | 42262905 | 143 | chr19 | 51959854 | 51961179 | 1326 |
| chr19 | 42267234 | 42267557 | 324 | chr19 | 51987046 | 51989084 | 2039 |
| chr19 | 42290598 | 42291224 | 627 | chr19 | 51991795 | 51995643 | 3849 |
| chr19 | 42827772 | 42828087 | 316 | chr19 | 51998370 | 51999259 | 890 |
| chr19 | 43174426 | 43174763 | 338 | chr19 | 52012332 | 52012665 | 334 |
| chr19 | 43259519 | 43260388 | 870 | chr19 | 52032107 | 52032340 | 234 |
| chr19 | 43393776 | 43394263 | 488 | chr19 | 52033987 | 52034233 | 247 |
| chr19 | 43404148 | 43405131 | 984 | chr19 | 52040560 | 52041629 | 1070 |
| chr19 | 43654179 | 43654620 | 442 | chr19 | 52045441 | 52045889 | 449 |
| chr19 | 43791349 | 43792685 | 1337 | chr19 | 52047882 | 52048980 | 1099 |
| chr19 | 45029044 | 45029637 | 594 | chr19 | 52065507 | 52067757 | 2251 |
| chr19 | 45041177 | 45041362 | 186 | chr19 | 52147538 | 52147851 | 314 |
| chr19 | 45043331 | 45043600 | 270 | chr19 | 52206501 | 52206788 | 288 |
| chr19 | 45044725 | 45045054 | 330 | chr19 | 52302389 | 52302593 | 205 |
| chr19 | 46282478 | 46282652 | 175 | chr19 | 52996020 | 52996710 | 691 |
| chr19 | 46727029 | 46727253 | 225 | chr19 | 53775393 | 53776147 | 755 |
| chr19 | 46992358 | 46992538 | 181 | chr19 | 54192299 | 54192389 | 91 |
| chr19 | 46996404 | 46998156 | 1753 | chr19 | 54203419 | 54204165 | 747 |
| chr19 | 48459303 | 48459452 | 150 | chr19 | 54317851 | 54318083 | 233 |
| chr19 | 48594599 | 48594734 | 136 | chr19 | 54325357 | 54325814 | 458 |
| chr19 | 48983665 | 48984248 | 584 | chr19 | 54337102 | 54337204 | 103 |
| chr19 | 54394939 | 54395265 | 327 | chr19 | 55212619 | 55212968 | 350 |
| chr19 | 54505426 | 54507013 | 1588 | chr19 | 55216943 | 55217277 | 335 |
| chr19 | 54519552 | 54519987 | 436 | chr19 | 55218367 | 55221632 | 3266 |
| chr19 | 54521544 | 54522904 | 1361 | chr19 | 55236907 | 55237364 | 458 |
| chr19 | 54561683 | 54565097 | 3415 | chr19 | 55239046 | 55239342 | 297 |
| chr19 | 54576156 | 54576429 | 274 | chr19 | 55241164 | 55241348 | 185 |
| chr19 | 54742837 | 54743057 | 221 | chr19 | 55242382 | 55242925 | 544 |
| chr19 | 54748112 | 54748312 | 201 | chr19 | 55254944 | 55258895 | 3952 |
| chr19 | 54759356 | 54762960 | 3605 | chr19 | 55267541 | 55267824 | 284 |
| chr19 | 54764160 | 54766198 | 2039 | chr19 | 55273520 | 55273982 | 463 |
| chr19 | 54775151 | 54777545 | 2395 | chr19 | 55294382 | 55295815 | 1434 |
| chr19 | 54780739 | 54782829 | 2091 | chr19 | 55296874 | 55303207 | 6334 |
| chr19 | 54785444 | 54785725 | 282 | chr19 | 55316444 | 55316992 | 549 |
| chr19 | 54792699 | 54794022 | 1324 | chr19 | 55329145 | 55331458 | 2314 |
| chr19 | 54795905 | 54796492 | 588 | chr19 | 55363514 | 55365348 | 1835 |
| chr19 | 54798272 | 54798649 | 378 | chr19 | 55369445 | 55371213 | 1769 |
| chr19 | 54800444 | 54802576 | 2133 | chr19 | 55377343 | 55378857 | 1515 |
| chr19 | 54803952 | 54804588 | 637 | chr19 | 55385587 | 55385745 | 159 |
| chr19 | 54823158 | 54823844 | 687 | chr19 | 55397786 | 55398013 | 228 |
| chr19 | 54840953 | 54841339 | 387 | chr19 | 55464247 | 55464333 | 87 |
| chr19 | 54842919 | 54843212 | 294 | chr19 | 55469305 | 55469773 | 469 |
| chr19 | 54844609 | 54845713 | 1105 | chr19 | 55506748 | 55508067 | 1320 |
| chr19 | 54847230 | 54848122 | 893 | chr19 | 55630211 | 55630261 | 51 |
| chr19 | 54849249 | 54850942 | 1694 | chr19 | 56226788 | 56226965 | 178 |
| chr19 | 54859084 | 54861102 | 2019 | chr19 | 56228726 | 56229056 | 331 |
| chr19 | 54868178 | 54868955 | 778 | chr19 | 56237483 | 56237642 | 160 |
| chr19 | 54935542 | 54935577 | 36 | chr19 | 56247146 | 56247346 | 201 |
| chr19 | 54937794 | 54938037 | 244 | chr19 | 56249720 | 56250384 | 665 |
| chr19 | 54996289 | 54996449 | 161 | chr19 | 56257394 | 56258113 | 720 |
| chr19 | 55010247 | 55010861 | 615 | chr19 | 56277733 | 56278241 | 509 |
| chr19 | 55015275 | 55015529 | 255 | chr19 | 56281625 | 56281828 | 204 |
| chr19 | 55044542 | 55045791 | 1250 | chr19 | 56332270 | 56332786 | 517 |
| chr19 | 55047309 | 55047765 | 457 | chr19 | 56354404 | 56354919 | 516 |
| chr19 | 55086103 | 55088276 | 2174 | chr19 | 56399756 | 56400004 | 249 |
| chr19 | 55100201 | 55100841 | 641 | chr19 | 56412609 | 56413544 | 936 |
| chr19 | 55103438 | 55107748 | 4311 | chr19 | 56416678 | 56417442 | 765 |
| chr19 | 55109072 | 55109898 | 827 | chr19 | 56459792 | 56460503 | 712 |
| chr19 | 55112041 | 55113983 | 1943 | chr19 | 56463770 | 56464350 | 581 |
| chr19 | 55117361 | 55118070 | 710 | chr19 | 56511931 | 56512245 | 315 |
| chr19 | 55120403 | 55120949 | 547 | chr19 | 56536916 | 56537374 | 459 |
| chr19 | 55140449 | 55141029 | 581 | chr19 | 56549015 | 56550139 | 1125 |
| chr19 | 55142480 | 55142867 | 388 | chr19 | 56565358 | 56565793 | 436 |
| chr19 | 55144118 | 55147238 | 3121 | chr19 | 57375843 | 57375977 | 135 |
| chr19 | 55148246 | 55148629 | 384 | chr19 | 58554569 | 58554633 | 65 |
| chr19 | 55154822 | 55155849 | 1028 | chr19 | 58715322 | 58715946 | 625 |
| chr19 | 55167678 | 55167882 | 205 | chr19 | 58728421 | 58728545 | 125 |
| chr19 | 55175616 | 55176342 | 727 | chr19 | 58867644 | 58868122 | 479 |
| chr19 | 55177868 | 55178957 | 1090 | chr20 | 821798 | 822866 | 1069 |
| chr19 | 55193024 | 55193238 | 215 | chr20 | 1502006 | 1502894 | 889 |
| chr20 | 1650018 | 1650712 | 695 | chr20 | 61715715 | 61715783 | 69 |
| chr20 | 1670835 | 1671631 | 797 | chr20 | 61719298 | 61721335 | 2038 |
| chr20 | 1705942 | 1707130 | 1189 | chr20 | 61728474 | 61728795 | 322 |
| chr20 | 1716388 | 1716411 | 24 | chr20 | 61750576 | 61751605 | 1030 |
| chr20 | 2061608 | 2062172 | 565 | chr20 | 61757524 | 61759193 | 1670 |
| chr20 | 9488329 | 9488990 | 662 | chr20 | 61760377 | 61760583 | 207 |
| chr20 | 9497095 | 9497159 | 65 | chr20 | 61770280 | 61770372 | 93 |
| chr20 | 17850931 | 17851008 | 78 | chr20 | 61783205 | 61783263 | 59 |
| chr20 | 20345939 | 20346140 | 202 | chr20 | 61787602 | 61787835 | 234 |
| chr20 | 20973396 | 20973760 | 365 | chr20 | 61793515 | 61794273 | 759 |
| chr20 | 21424972 | 21425361 | 390 | chr20 | 62034458 | 62034606 | 149 |
| chr20 | 21560630 | 21561574 | 945 | chr20 | 62088162 | 62088462 | 301 |
| chr20 | 21589123 | 21589261 | 139 | chr20 | 62114890 | 62114948 | 59 |
| chr20 | 21605440 | 21606028 | 589 | chr20 | 62171845 | 62172230 | 386 |
| chr20 | 21655571 | 21656122 | 552 | chr20 | 62369103 | 62369339 | 237 |
| chr20 | 22216869 | 22216931 | 63 | chr20 | 62573257 | 62573704 | 448 |
| chr20 | 22246512 | 22247855 | 1344 | chr21 | 9854227 | 9854794 | 568 |
| chr20 | 25838961 | 25839309 | 349 | chr21 | 9871411 | 9872434 | 1024 |
| chr20 | 29973627 | 29974101 | 475 | chr21 | 10027299 | 10027981 | 683 |
| chr20 | 29979461 | 29980097 | 637 | chr21 | 10792947 | 10793248 | 302 |
| chr20 | 30815529 | 30816031 | 503 | chr21 | 30502797 | 30503084 | 288 |
| chr20 | 33585138 | 33585201 | 64 | chr21 | 31830342 | 31830442 | 101 |
| chr20 | 34253287 | 34253386 | 100 | chr21 | 32253316 | 32253627 | 312 |
| chr20 | 35490058 | 35490780 | 723 | chr21 | 32308657 | 32309300 | 644 |
| chr20 | 36265427 | 36265603 | 177 | chr21 | 33628803 | 33628880 | 78 |
| chr20 | 39311925 | 39312435 | 511 | chr21 | 36612130 | 36612391 | 262 |
| chr20 | 41193758 | 41194095 | 338 | chr21 | 36936600 | 36936780 | 181 |
| chr20 | 42135843 | 42135906 | 64 | chr21 | 38366920 | 38367013 | 94 |
| chr20 | 43726501 | 43726661 | 161 | chr21 | 38378066 | 38378272 | 207 |
| chr20 | 44168796 | 44169747 | 952 | chr21 | 40145361 | 40145426 | 66 |
| chr20 | 44182779 | 44183632 | 854 | chr21 | 40192474 | 40192901 | 428 |
| chr20 | 44308895 | 44309875 | 981 | chr21 | 41047974 | 41048161 | 188 |
| chr20 | 47025966 | 47026300 | 335 | chr21 | 41425198 | 41425428 | 231 |
| chr20 | 47117778 | 47118018 | 241 | chr21 | 42053181 | 42053754 | 574 |
| chr20 | 49245004 | 49245058 | 55 | chr21 | 44699071 | 44699124 | 54 |
| chr20 | 55412550 | 55413364 | 815 | chr21 | 44789157 | 44790154 | 998 |
| chr20 | 55964919 | 55964992 | 74 | chr21 | 44863888 | 44864005 | 118 |
| chr20 | 56725871 | 56726058 | 188 | chr21 | 45968434 | 45968589 | 156 |
| chr20 | 57056677 | 57056850 | 174 | chr21 | 45971035 | 45971249 | 215 |
| chr20 | 57089461 | 57090257 | 797 | chr21 | 45977985 | 45978031 | 47 |
| chr20 | 57580191 | 57580399 | 209 | chr21 | 46036079 | 46036560 | 482 |
| chr20 | 58790682 | 58791080 | 399 | chr21 | 46475796 | 46475956 | 161 |
| chr20 | 59552956 | 59553162 | 207 | chr21 | 47009849 | 47010268 | 420 |
| chr20 | 59641612 | 59642132 | 521 | chr21 | 47035252 | 47036261 | 1010 |
| chr20 | 60203546 | 60203770 | 225 | chr22 | 16871435 | 16871577 | 143 |
| chr20 | 61307217 | 61307417 | 201 | chr22 | 17041693 | 17042671 | 979 |
| chr20 | 61662059 | 61662112 | 54 | chr22 | 17048747 | 17049383 | 637 |
| chr20 | 61669257 | 61669889 | 633 | chr22 | 17065795 | 17065952 | 158 |
| chr20 | 61675521 | 61675607 | 87 | chr22 | 17180149 | 17180381 | 233 |
| chr22 | 17229383 | 17229424 | 42 | | | | |
| chr22 | 17340382 | 17341138 | 757 | | | | |
| chr22 | 18038291 | 18038474 | 184 | | | | |
| chr22 | 18769671 | 18769779 | 109 | | | | |
| chr22 | 19020548 | 19020625 | 78 | | | | |
| chr22 | 19142379 | 19142770 | 392 | | | | |
| chr22 | 19154221 | 19155374 | 1154 | | | | |
| chr22 | 19280049 | 19280080 | 32 | | | | |
| chr22 | 19709131 | 19712064 | 2934 | | | | |
| chr22 | 20780208 | 20780439 | 232 | | | | |
| chr22 | 20783550 | 20784375 | 826 | | | | |
| chr22 | 21823809 | 21823902 | 94 | | | | |
| chr22 | 22479786 | 22480080 | 295 | | | | |
| chr22 | 22611749 | 22611840 | 92 | | | | |
| chr22 | 23799363 | 23799535 | 173 | | | | |
| chr22 | 24180627 | 24180665 | 39 | | | | |
| chr22 | 24890467 | 24891032 | 566 | | | | |
| chr22 | 26969305 | 26969692 | 388 | | | | |
| chr22 | 27536088 | 27536314 | 227 | | | | |
| chr22 | 29828210 | 29828531 | 322 | | | | |
| chr22 | 30476149 | 30476508 | 360 | | | | |
| chr22 | 32601042 | 32602589 | 1548 | | | | |
| chr22 | 32928726 | 32928838 | 113 | | | | |
| chr22 | 33468660 | 33469078 | 419 | | | | |
| chr22 | 35195903 | 35196533 | 631 | | | | |
| chr22 | 35435986 | 35436242 | 257 | | | | |
| chr22 | 36806279 | 36806402 | 124 | | | | |
| chr22 | 37471214 | 37471339 | 126 | | | | |
| chr22 | 37838107 | 37838393 | 287 | | | | |
| chr22 | 38478373 | 38478751 | 379 | | | | |
| chr22 | 38965701 | 38966026 | 326 | | | | |
| chr22 | 39192005 | 39192642 | 638 | | | | |
| chr22 | 40058693 | 40058844 | 152 | | | | |
| chr22 | 40391008 | 40391437 | 430 | | | | |
| chr22 | 40781546 | 40781783 | 238 | | | | |
| chr22 | 41047867 | 41048665 | 799 | | | | |
| chr22 | 42095614 | 42095657 | 44 | | | | |
| chr22 | 45196738 | 45197232 | 495 | | | | |
| chr22 | 45233398 | 45233566 | 169 | | | | |
| chr22 | 46451704 | 46453337 | 1634 | | | | |
| chr22 | 46474921 | 46477355 | 2435 | | | | |
| chr22 | 49764866 | 49765130 | 265 | | | | |
| chr22 | 50055234 | 50055477 | 244 | | | | |
| chr22 | 50466160 | 50466419 | 260 | | | | |
| chr22 | 50528381 | 50528466 | 86 | | | | |
| chr22 | 50623433 | 50623693 | 261 | | | | |
| chr22 | 50681372 | 50681831 | 460 | | | | |
| chr22 | 51016421 | 51016639 | 219 | | | | |
| chr22 | 51042329 | 51043071 | 743 | | | | |

After subjecting the nucleic acid molecules or derivatives thereof to sequencing, suitable bioinformatics processes may be performed on the sequence reads to generate the data set comprising the methylation pattern of one or more target regions of the DNA sample. For example, sequence reads may be aligned to one or more reference genomes (e.g., a human genome). The aligned sequence reads may be quantified at one or more genomic loci or target regions to generate the data set comprising the methylation pattern profile of one or more target regions of the cell-free biological sample. Quantification of sequences may be expressed as un-normalized or normalized values.

In some embodiments. Alignment of bisulfite converted DNA is performed using a software program such as Bismark (Krueger et al. (2011) Bioinformatics, 27(11): 157171). Bismark performs both read mapping and methylation calling in a single step and its output discriminates between cytosines in CpG, CHG and CHH contexts. Bismark is released under the GNU GPLv3+ license. The source code is freely available at bioinformatics.bbsrc.ac.uk/projects/bismark/. In some embodiments, differential methylation is calculated for specific loci/regions using, for example, one or more publicly available programs to analyze and/or determine methylation levels or a target polynucleotide region. In some embodiments, the method used to analyze and/or determine methylation levels of a target polynucleotide region include Metilene (Juhling et al., Genome Res., 2016; 26(2): 256-262) or GenomeStudio Software available online from Illumina, Inc. Other methods of determining differentially methylated target polynucleotide regions are described in Hovestadt et al., 2014; Nature, 510(7506), 537-541.

In some embodiments, the target regions that are examined to determine the presence or absence of a pediatric cancer in a subject comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target regions listed in Table 1.

In some embodiments, the target regions that are examined to determine the severity of a pediatric cancer (i.e., stage I, stage II, stage III, or stage IV cancer) subject comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target regions listed in Table 1.

In some embodiments, the target genomic regions that are examined to determine the presence or absence of an adult cancer in a subject comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target regions listed in Table 1.

In some embodiments, the target regions that are examined to determine the severity of a an adult cancer (i.e., stage I, stage II, stage III, or stage IV cancer) subject comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target regions listed in Table 1.

Target genomic regions that are examined to determine the presence of MRD in a subject comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target regions listed in Table 1.

In some embodiments, the biological sample may be collected through a standard biopsy or a liquid biopsy. Preferably, the biopsy is a liquid biopsy and the cfDNA may be collected from whole blood, plasma, serum, or urine. In some embodiments, an amount of sample, such as whole blood, may include an amount of about 50 μL to about 5 mL, about 100 μL to about 5 mL, about 150 μL to about 5 mL, about 200 μL to about 5 mL, about 250 μL to about 5 mL, about 300 μL to about 5 mL, about 350 μL to about 5 mL, about 400 μL to about 5 mL, about 450 μL to about 5 mL, about 500 μL to about 5 mL, about 550 μL to about 5 mL, about 600 μL to about 5 mL, about 700 μL to about 5 mL, about 750 μL to about 5 mL, about 800 μL to about 5 mL, about 850 μL to about 5 mL, about 900 μL to about 5 mL, about 950 μL to about 5 mL, about 1 mL to about 5 mL, about 1.5 mL to about 5 mL, about 2 mL to about 5 mL, about 2.5 mL to about 5 mL, or about 3 mL to about 5 mL. In another embodiment, an amount of sample, such as whole blood, may include an amount of about 5 mL to about 10 mL.

Isolation and extraction of DNA, and in particular, cfDNA, may be performed through collection of bodily fluids using a variety of techniques. In some cases, collection may comprise aspiration of a bodily fluid from a subject using a syringe. In other cases, collection may comprise pipetting or direct collection of fluid into a collecting vessel. Methods for isolating DNA or other nucleic acids from tissue samples are well-known in the art.

After collection of bodily fluid, cfDNA may be isolated and extracted using a variety of techniques known to a person of ordinary skill in the art. In some cases, cell-free nucleic acid may be isolated, extracted and prepared using commercially available kits such as the Qiagen Qiamp® Circulating Nucleic Acid Kit protocol. In other examples, Qiagen Qubit™ dsDNA HS Assay kit protocol, Agilent™ DNA 1000 kit, or TruSeq™ Sequencing Library Preparation; Low-Throughput (LT) protocol.

Alternatively, cfDNA may be extracted and isolated by from bodily fluids through a partitioning step in which cfDNAs, as found in solution, are separated from cells and other non-soluble components of the bodily fluid. Partitioning may include, but is not limited to, techniques such as centrifugation or filtration. In other cases, cells may not be partitioned from cfDNA first, but rather lysed. For instance, the genomic DNA of intact cells may be partitioned through selective precipitation.

In some embodiments, the pediatric cancer or adult cancer is osteosarcoma, medulloblastomas, ependymomas, optical nerve gliomas, brain stem glioma, oligodendrogliomas, gangliogliomas. Pineal Region Tumors, hepatoblastoma, Fibrolamellar Hepatocellular Carcinoma, Hodgkin's lymphoma, Non-Hodgkin's lymphoma, acute lymphoblastic leukemia, acute myeloid leukemia, juvenile myelomonocytic leukemia, acute promyelocytic leukemia, chronic lymphoblastic leukemia, chronic myeloid leukemia, diffuse intrinsic pontine glioma, neuroblastoma, retinoblastoma, rhaboid tumors, Ewing's sarcoma, rhabdomyosarcoma, embryonal rhabdomyosarcoma, fibrosarcoma, mesenchymoma, synovial sarcoma, a teratoma, liposarcoma, spinal cord tumors, ovarian cancer, or Wilm's tumors. In some embodiments, the cancers are recurrent cancers (e.g., recurrent ovarian cancer, recurrent teratoma, recurrent Wilm's tumors, etc.)

In some embodiments, the pediatric or adult cancer is a central nervous system cancer such as anaplastic pilocytic astrocytoma; atypical teratoid/rhabdoid tumor, subclass MYC; atypical teratoid/rhabdoid tumor, subclass SHH; atypical teratoid/rhabdoid tumor, subclass TYR; cerebellar liponeurocytoma; CNS Ewing sarcoma family tumor with CIC alteration; CNS high grade neuroepithelial tumor with BCOR alteration: CNS high grade neuroepithelial tumor with MN1 alteration: CNS neuroblastoma with FOXR2 activation; diffuse leptomeningeal glioneuronal tumor; diffuse midline glioma H3 K27M mutant; embryonal tumor with multilayered rosettes; ependymoma, posterior fossa group A: ependymoma, RELA fusion; esthesioncuroblastoma, subclass A; glioblastoma, IDH wildtype, H3.3 G34 mutant; glioblastoma. IDH wildtype, subclass mesenchymal; glioblastoma, IDH wildtype, subclass midline; glioblastoma, IDH wildtype, subclass MYCN; glioblastoma. IDH wildtype, subclass RTK I; glioblastoma, IDH wildtype, subclass RTK II; glioblastoma, IDH wildtype, subclass RTK III; IDH glioma, subclass 1p/19q co-deleted oligodendroglioma; IDH glioma, subclass astrocytoma; IDH glioma, subclass high grade astrocytoma; low grade glioma, MYB/MYBL1; low grade glioma, rosette forming glioneuronal tumor: lymphoma; medulloblastoma, subclass group 3; medulloblastoma, subclass group 4; medulloblastoma, subclass SHH A (children and adult); medulloblastoma, subclass SHH B (infant); medulloblastoma, WNT: melanoma:papillary tumor of the pineal region group B; pineal parenchymal tumor; pineoblastoma group A/intracranial retinoblastoma; pineoblastoma group B; plexus tumor, subclass pediatric B; retinoblastoma; or subependymoma, spinal. In some embodiments, the CNS cancers are recurrent cancers.

In some embodiments, the pediatric or adult cancer is Bladder Urothelial Carcinoma, Breast invasive carcinoma, Colon adenocarcinoma, Esophageal carcinoma. Head and Neck squamous cell carcinoma, Kidney renal clear cell carcinoma, Kidney renal papillary cell carcinoma, Liver hepatocellular carcinoma, Lung adenocarcinoma, Lung squamous cell carcinoma, Pancreatic adenocarcinoma, Prostate adenocarcinoma, Thyroid carcinoma, Uterine Corpus Endometrial Carcinoma. In some embodiments, the cancer is breast cancer. In some embodiments, the cancers are recurrent cancers (e.g., recurrent colon adenocarcinoma, recurrent esophageal carcinoma, recurrent lung adenocarcinoma, etc.).

Upon identifying a subject as having, for example, a pediatric cancer, an adult cancer, or MRD, a clinical procedure or cancer therapy can be administered to the subject. Exemplary therapies or procedures include but are not limited to surgery, radiation therapy, chemotherapy, hormone therapy, targeted therapy, and/or administration of an effective mount of one or more therapeutic agents: angiogenesis inhibitors, such as angiostatin K1-3, DL-α-Difluoromethyl-omithine, endostatin, fumagillin, genistein, minocycline, staurosporine, and (±)-thalidomide; DNA intercalator/cross-linkers, such as Bleomycin, Carboplatin, Carmustine. Chlorambucil, Cyclophosphamide, cis-Diammineplatinum(II) dichloride (Cisplatin), Melphalan, Mitoxantrone, and Oxaliplatin; DNA synthesis inhibitors, such as (±)-Amethopterin (Methotrexate), 3-Amino-1,2,4-benzotriazine 1,4-dioxide, Aminopterin, Cytosine β-D-arabinofuranoside, 5-Fluoro-5′-deoxyuridine, 5-Fluorouracil, Ganciclovir, Hydroxyurea, and Mitomycin C; DNA-RNA transcription regulators, such as Actinomycin D, Daunorubicin, Doxorubicin, Homoharringtonine, and Idarubicin; enzyme inhibitors, such as S(+)-Camptothecin, Curcumin, (−)-Deguelin, 5,6-Dichlorobenzimidazole 1-β-D-ribofuranoside, Etoposide, Formestane. Fostriecin. Hispidin, 2-Imino-1-imidazoli-dineacetic acid (Cyclocreatine), Mevinolin, Trichostatin A, Tyrphostin AG 34, and Tyrphostin AG 879; gene regulators, such as 5-Aza-2′-deoxycytidine, 5-Azacytidine, Cholecalciferol (Vitamin D3), 4-Hydroxytamoxifen, Melatonin, Mifepristone, Raloxifene, all trans-Retinal (Vitamin A aldehyde), Retinoic acid, all trans (Vitamin A acid), 9-cis-Retinoic Acid, 13-cis-Retinoic acid, Retinol (Vitamin A), Tamoxifen, and Troglitazone; microtubule inhibitors, such as Colchicine, Dolastatin 15, Nocodazole, Paclitaxel, Podophyllotoxin, Rhizoxin, Vinblastine, Vincristine, Vindesine, and Vinorelbine (Navelbine); and unclassified antitumor agents, such as 17-(Allylamino)-17-demethoxygeldanamycin, 4-Amino-1,8-naphthalimide, Apigenin, Brefeldin A, Cimetidine, Dichloromethylene-diphosphonic acid. Leuprolide (Leuprorelin), Luteinizing Hormone-Releasing Hormone, Pifithrin-α, Rapamycin, Sex hormone-binding globulin, Thapsigargin, and Urinary trypsin inhibitor fragment (Bikunin). The antitumor agent may be a neoantigen. Neoantigens are tumor-associated peptides that serve as active pharmaceutical ingredients of vaccine compositions which stimulate antitumor responses and are described in US Pub. No. 2011/0293637, which is incorporated by reference herein in its entirety. The antitumor agent may be a monoclonal antibody such as rituximab, alemtuzumab, Ipilimumab, Bevacizumab, Cetuximab, panitumumab, and trastuzumab, Vemurafenib imatinib mesylate, erlotinib, gefitinib, Vismodegib, 90Y-ibritumomab tiuxetan, ¹³¹I-tositumomab, ado-trastuzumab emtansine, lapatinib, pertuzumab, ado-trastuzumab emtansine, regorafenib, sunitinib, Denosumab, sorafenib, pazopanib, axitinib, dasatinib, nilotinib, bosutinib, ofatumumab, obinutuzumab, ibrutinib, idelalisib, crizotinib, erlotinib (Tarceva®), afatinib dimaleate, ceritinib, Tositumomab and ¹³¹I-tositumomab, ibritumomab tiuxetan, brentuximab vedotin, bortezomib, siltuximab, trametinib, dabrafenib, pembrolizumab, carfilzomib, Ramucirumab, Cabozantinib, vandetanib, The antitumor agent may be a cytokine such as interferons (INFs), interleukins (ILs), or hematopoietic growth factors. The antitumor agent may be INF-α, IL-2, Aldesleukin, IL-2, Erythropoietin, Granulocyte-macrophage colony-stimulating factor (GM-CSF) or granulocyte colony-stimulating factor. The antitumor agent may be a targeted therapy such as toremifene, fulvestrant, anastrozole, exemestane, letrozole, ziv-aflibercept, Alitretinoin, temsirolimus, Tretinoin, denileukin diftitox, vorinostat, romidepsin, bexarotene, pralatrexate, lenaliomide, belinostat, pomalidomide, Cabazitaxel, enzalutamide, abiraterone acetate, ²²³radium chloride, or everolimus. The antitumor agent may be a checkpoint inhibitor such as an inhibitor of the programmed death-1 (PD-1) pathway, for example an anti-PD1 antibody (Nivolumab). The inhibitor may be an anti-cytotoxic T-lymphocyte-associated antigen (CTLA-4) antibody. The inhibitor may target another member of the CD28 CTLA4 Ig superfamily such as BTLA, LAG3, ICOS, PDL1 or KIR. A checkpoint inhibitor may target a member of the TNFR superfamily such as CD40, OX40, CD137, GITR, CD27 or TIM-3. Additionally, the antitumor agent may be an epigenetic targeted drug such as HDAC inhibitors, kinase inhibitors, DNA methyltransferase inhibitors, histone demethylase inhibitors, or histone methylation inhibitors. The epigenetic drugs may be Azacitidine, Decitabine, Vorinostat, Romidepsin, or Ruxolitinib.

In some embodiments, method of treatment of a pediatric cancer, an adult cancer, or MRD may include administration of an effective amount of a suitable substance able to target intracellular proteins, small molecules, or nucleic acid molecules alone or in combination with an appropriate carrier or vehicle, including, but not limited to, an antibody or functional fragment thereof, (e.g., Fab′, F(ab′)2, Fab, Fv, rlgG, and scFv fragments and genetically engineered or otherwise modified forms of immunoglobulins such as intrabodies and chimeric antibodies), small molecule inhibitors of the protein, chimeric proteins or peptides, gene therapy for inhibition of transcription, or an RNA interference (RNAi)-related molecule or morpholino molecule able to inhibit gene expression and/or translation. In one embodiment the inhibitor is an RNAi-related molecule such as an siRNA or an shRNA for inhibition of translation. An RNA interference (RNAi) molecule is a small nucleic acid molecule, such as a short interfering RNA (siRNA), a double-stranded RNA (dsRNA), a micro-RNA (miRNA), or a short hairpin RNA (shRNA) molecule, that complementarily binds to a portion of a target gene or mRNA so as to provide for decreased levels of expression of the target.

Suitable pharmaceutical composition comprising one or more of the agents described herein is administered and dosed in accordance with good medical practice, taking into account the clinical condition of the individual patient, the site and method of administration, scheduling of administration, patient age, sex, body weight, and other factors known to medical practitioners. The therapeutically effective amount for purposes herein is thus determined by such considerations as are known in the art. For example, an effective amount of the pharmaceutical composition is that amount necessary to provide a therapeutically effective decrease in the expression of the targeted gene. The amount of the pharmaceutical composition should be effective to achieve improvement including but not limited to total prevention and to improved survival rate or more rapid recovery, or improvement or elimination of symptoms associated with the chronic inflammatory conditions being treated and other indicators as are selected as appropriate measures by those skilled in the art. In accordance with the present technology, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of the patient and the route of administration.

The pharmaceutical compositions can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the technology. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils.

Compositions containing pharmaceutically acceptable carriers are described in several resources which are well known and readily available to those skilled in the art. For example, Remington: The Science and Practice of Pharmacy (Gerbino, P. P. [2005] Philadelphia, Pa., Lippincott Williams & Wilkins, 21 st ed.) describes formulations that can be used in connection with the subject technology. Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. In addition to the ingredients particularly mentioned above, the formulations of the subject technology can include other agents conventional in the art having regard to the type of formulation in question.

The disclosure also provides for assay panels (nucleic acid hybridization probes or sets of probes) comprises a plurality of polynucleotide probes, wherein each of the polynucleotide probes is configured to hybridize to a bisulfate-converted fragment obtained from processing of DNA, or more preferably, cfDNA molecules, from a subject, wherein each of the cfDNA molecules corresponds to or is derived from, or includes the one or more target regions selected from Table 1.

In some embodiments, the methods described herein also may be implemented by use of computer systems. For example, any of the steps described above for evaluating sequence reads to determine methylation status of a CpG site may be performed by means of software components loaded into a computer or other information appliance or digital device. When so enabled, the computer, appliance or device may then perform all or some of the above-described steps to assist the analysis of values associated with the methylation of a one or more CpG sites, or for comparing such associated values. The above features embodied in one or more computer programs may be performed by one or more computers running such programs.

Further, various aspects of the methods disclosed herein can be implemented using computer-based calculations, machine learning (e.g., support vector machine (SVM), Lasso, Generalized Linear Model (GLM), Gradient Boosted Model (GBM), Extreme Gradient Boosting (XGB), Elastic-Net Regularized Generalized Linear Models (Glmnet), Random Forest, Gradient boosting (on random forest), C5.0 decision trees), and other software tools, or combinations thereof. For example, a methylation status for a CpG site can be assigned by a computer based on an underlying sequence read of an amplicon from a sequencing assay. In another example, a methylation value for a DNA region or portion thereof can be compared by a computer to a threshold value, as described herein. The tools are advantageously provided in the form of computer programs that are executable by a general-purpose computer system of conventional design.

In some embodiments, the method used to analyze and/or determine methylation levels of a target polynucleotide region includes Metilene (Juhling et al., Genome Res., 2016; 26(2): 256-262) or GenomeStudio Software available online from Illumina, Inc., or as described in Hovestadt et al., 2014; Nature, 510(7506), 537-541. In some embodiments, methylation data may be further processed by algorithms and/or software to determine the differential values (i.e. differential methylation value) and identify differentially methylated regions (DMRs). Differential methylation value may be calculated by methods known in the art (see, e.g. Hovestadt, et al. (2014). Nature, 510(7506), 537-541). In some embodiments, Metilene, a software program for calling differentially methylated regions may be used to identify differentially methylated regions within whole genome and targeted sequencing data. In some embodiments, the methylation data may be divided into hypomethylated target regions, hypermethylated target regions, a combination of both hypo and hypermethylated regions. In some embodiments, the machine learning model may be trained using hypomethylated target regions, hypermethylated target regions, or both.

In some embodiments, methods of identifying a pediatric cancer, an adult cancer, or MRD in a subject may comprise the use of a machine learning model. The machine learning model may be a trained algorithm. The machine learning model may be trained on one or more features and trained be used to process a data set generated via assaying nucleic acid molecules in a sample (e.g., cell-free biological sample), which data set comprises a methylation profile of one or more target genomic regions of the biological sample. Examples of machine learning models use and training of said machine learning model are described, for example in PCT Pub. No. WO2022/178108 to Salhia et al.; WO2019/178277 to Gross et al.; U.S. Pat. Pub No. US2021/00250 to Gross et al.; and U.S. Pat. Pub. No. US2019/0287652 to Gross et al. In some embodiments, the machine learning model is trained using samples of DNA comprising the target regions of Table 1 taken from subjects having a known cancer. In some embodiments, the machine learning model is trained using samples of 1, 2, 3, 4, 5, 6, 7, 8, 9, 10, 11, 12, 13, 14, 15, 16, 17, 18, 19, or 20 or more cancers.

In some embodiments, the target regions of the DNA isolated from a subject may be divided into hypomethylated target regions, hypermethylated target regions, or a combination thereof, and, for example, individually analyzed using the machine learning model. As another example, the target regions of DNA of the subject may be analyzed using a machine model trained solely on hypermethylated regions or hypomethylated regions.

In some embodiments, a computer comprising at least one processor may be configured to receive a plurality of sequencing results from the DNA methylation sequencing reactions (e.g., after WGBS) that may comprise the methylation pattern of one or more target regions disclosed herein (e.g., Table 1) from a patient having a mass or other tumor (e.g., DNA isolated form the mass or tumor, or DNA isolated from cfDNA from the person having the mass or tumor) or otherwise suspected of having a cancer. The methylation pattern of the received sequences reads may be determined through sequence alignment with a reference genome and, for example, using Metilene or other commercially available product, identifying differentially methylated regions between the sample and a normal sample or pool of normal samples. A trained machine learning model may be applied to these results to output a result (e.g., presence or absence of a pediatric or adult cancer, MRD, etc.). As used herein, “processor” may be any type of processor, such as, for example, any type of general-purpose microprocessor or microcontroller (e.g., an Intel™ x86, PowerPC™, ARM™ processor, or the like), a digital signal processing (DSP) processor, an integrated circuit, a field programmable gate array (FPGA), or any combination thereof.

In some embodiments, the machine learning model used to detect the pediatric cancer, adult cancer, or MRD comprises analyzes methylation patterns of a plurality of target regions of cancerous samples as compared to methylation patterns of a plurality of target regions of non-cancerous samples. In some embodiments, the pediatric cancer, adult cancer, and/or the MRD signature is detected by determining and analyzing a methylation pattern of a plurality of target regions of both cancerous and non-cancerous samples wherein the plurality of target regions comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target regions listed in Table 1. In some embodiments, the signature is developed by analyzing and comparing all the target regions of Table 1.

In some embodiments, the machine learning model is trained using target regions from a plurality of cancerous samples and corresponding target regions from non-cancerous samples, wherein the cancer samples comprise at least two different cancer types, wherein the detection of the pediatric cancer, the adult cancer, or the MRD is based on a comparison of a methylation pattern of target regions of the cancerous samples compared to a methylation pattern of corresponding target regions of the non-cancerous samples.

In some embodiments, the machine learning model is trained by aligning sequence reads of target regions of a plurality of cancer sample to the corresponding regions of a reference genome, then conducting methylation analysis of the target regions of the plurality of cancer samples to a normal sample pool or normal samples to identify the target regions that are differentially methylated: identifying the differentially methylated regions that are common between the cancer samples: using the common or shared differentially methylated regions to train the machine learning model to distinguish cancerous versus non-cancerous samples. In some embodiments, a target region of a sample having a methylation level that differs from the methylation level of the target sequences a normal sample is a differentially methylated region (DMR).

In some embodiments, sequence reads are aligned after whole genome sequencing (e.g., WGBS, RRBS). In some embodiments, training of the machine leaning model includes identifying the minimum number of differentially methylated regions that are shared across the plurality of cancer samples. In some embodiments, the minimally differentiated regions (mDMRs) are common to about 40% of the cancer samples, about 45% of the cancer samples, about 50% of the cancer samples, about 55% of the cancer samples, about 60% of the cancer samples, about 65% of the cancer samples, about 70% of the cancer samples, about 75% of the cancer samples, about 80% of the cancer samples, about 85% of the cancer samples, about 90% of the cancer samples, about 95% of the cancer samples, or greater than 95% of the cancer samples. In some embodiments, the mDMRs are common or shared between about 70% of the cancers from which the target samples are derived, about 75% of the cancers from which the target samples are derived, about 80% of the cancers from which the target samples are derived, about 85% of the cancers from which the target samples are derived, about 90% of the cancers from which the target samples are derived, about 95% of the cancers from which the target samples are derived, or about 100% of the cancers from which the target samples are derived.

In some embodiments, training a machine learning model comprises the steps of i) receiving methylation sequencing reads of a plurality of test samples including cancerous samples and non-cancerous samples to obtain a methylation pattern of target regions of the plurality of test samples, wherein the cancerous samples comprise at least two different cancer types; ii) aligning the target regions of the plurality of test samples with a reference genome, wherein each of the target regions of the plurality of test samples is aligned with a corresponding target region of the reference genome; iii) perform differentially methylated region analysis (e.g., using Metilene) of the test sample compared to a normal sample or pool of normal samples to identify differentially methylated regions between the test (cancer) samples and the normal samples; vi) define the minimally differentially methylated regions (mDMRs) across test samples; and vii) building a classifier model with mDMRs using machine learning tools to distinguish between cancerous and non-cancerous samples using the MDMRs wherein an AUC value of 0.8 or greater indicates the presence of the pediatric cancer, the adult cancer, or MRD. In some embodiments, step iii) further comprises determining hypomethylated target regions and hypomethylated target regions e.g., using a methylation caller (e.g., Metilene).

In some embodiments, a target region may be assigned a methylation value based on, for example, a positive number if the target region has a methylated CpG compared to the corresponding position of the reference genome (i.e., the CpG of the reference genome is unmethylated), and a negative number if the target region has a an unmethylated CpG compared to the corresponding position of the reference genome (i.e., the CpG of the reference genome is methylated). Thus, a hypermethylated target region may have a positive overall score and a hypomethylated may have a negative overall score.

In some embodiments, the output of the machine learning model comprises an Area Under the Curve (AUC) value for a test sample. In some embodiments, an AUC of 0.8 or greater, 0.85 or greater, 0.9 or greater, 0.95 or greater, 0.96 or greater, 0.97 or greater, 0.98 or greater, of 0.99 or greater indicates the presence of a pediatric cancer, adult cancer, or MRD.

In some embodiments, the first set of target regions, the second set of target regions, and the corresponding target regions comprise the same target regions of Table 1.

In some embodiments, the cancerous samples used to train the machine learning model comprise stage I-IV cancer samples, such as, for example, metastatic breast cancer. In other embodiments, the cancerous samples used to train the machine learning model are from stage I or stage II cancer samples, The methylation pattern of the cancerous samples may then be compared to the methylation pattern of the non-cancerous samples.

Any of the computer-readable media herein can be non-transitory (e.g., volatile memory such as DRAM or SRAM, nonvolatile memory such as magnetic storage, optical storage, or the like) and/or tangible. Any of the storing actions described herein can be implemented by storing in one or more computer-readable media (e.g., computer-readable storage media or other tangible media). Any of the things (e.g., data created and used during implementation) described as stored can be stored in one or more computer-readable media (e.g., computer-readable storage media or other tangible media). Computer-readable media can be limited to implementations not consisting of a signal.

In some embodiments, there is provided a kit for detecting a cancer, such as a pediatric cancer, adult cancer, or MRD comprising reagents for carrying out the aforementioned methods, and instructions for detecting the cancer signals. Reagents may include, for example, primer sets, PCR reaction components, a plurality of probe sets complementary to target regions of Table 1, sequencing reagents, and optionally, a solid support for said probes (e.g., a glass slide or chip, surface of a bead, surface of a matrix, etc.).

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### Examples

### Example 1

### Analysis of Differential DNA Methylation Patterns in Pediatric Cancers.

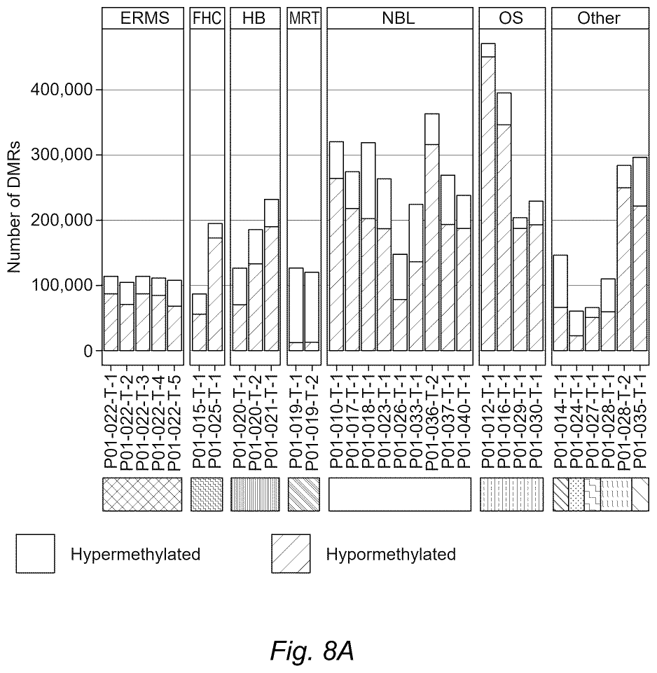

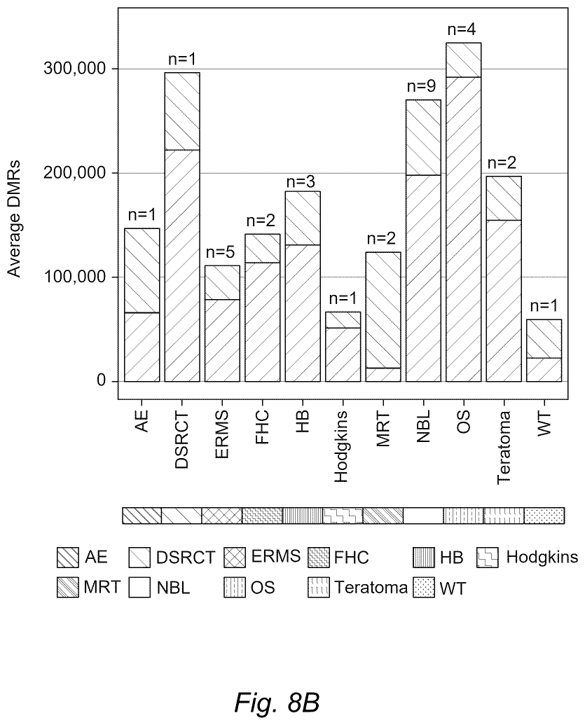

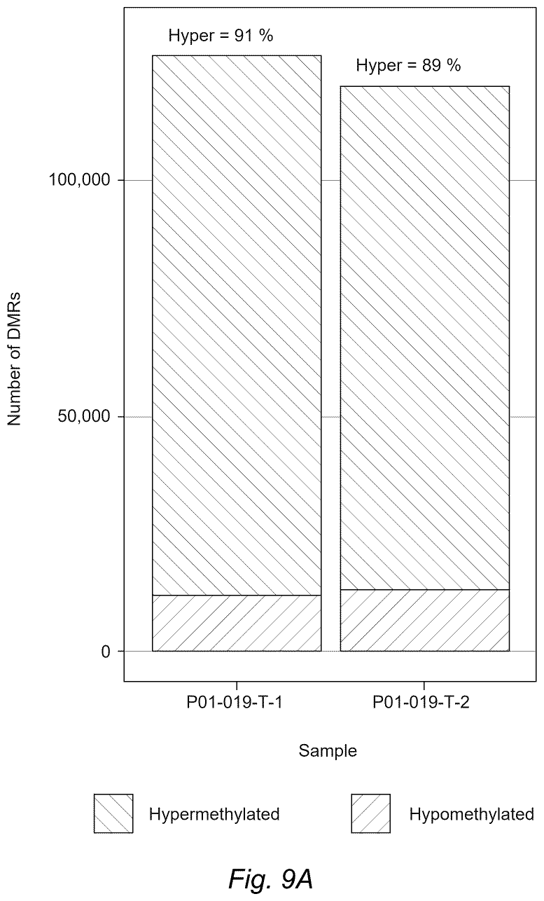

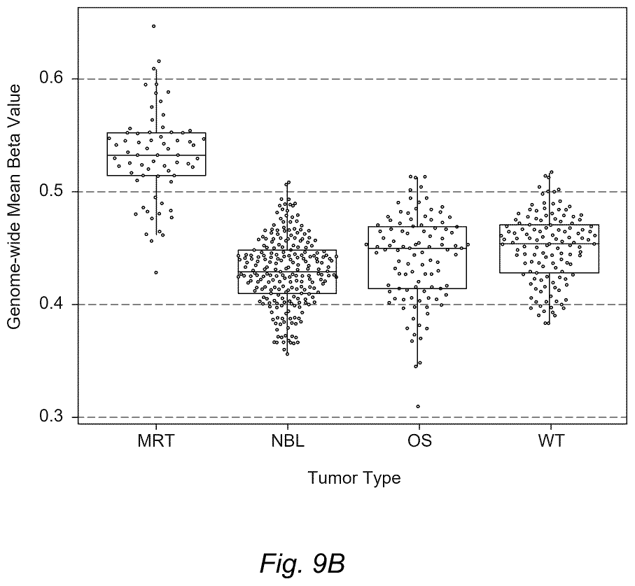

We performed WGBS on 31 tumor and 13 patient-matched adjacent normal tissue samples representing 11 different pediatric cancer types (Table 2). First, we performed differential methylation analysis using Metilene to find differentially methylated regions (DMRs); tumor samples were compared with their patient-matched adjacent normal sample where possible. For tumor samples without a matched normal, a pool of normal samples from other patients with the same diagnosis was used. Differential DNA methylation analysis revealed a variable number of DMRs both within and between tumor types (FIG. 8A-B, Table 3). On average, 74% of DMRs across all tumors were hypomethylated compared to 26% hypermethylated DMRs. However, malignant rhabdoid tumors (MRT) displayed a hypermethylator phenotype where 9000 of DMRs were hypermethylated (FIG. 9A). Global hypermethylation in MRT was also observed in 68 TARGET samples (FIG. 9B).

TABLE 3
DMR summaries from tissue samples. The average, median, minimum, and maximum
number of DMRs by cancer type. The number of cases for each type is also indicated.
The average number of CpGs, median width for the DMRs, and locations with respect
to CpG islands, shores, shelves and open sea are also indicated.

| Dx | nCases | mean.nDMR | median.nDMR | min.nDMR | max.nDMR | mean.nCpG |
|---|---|---|---|---|---|---|
| AE | 1 | 146689 | 146689 | 146689 | 146689 | 13.54663949 |
| DSRCT | 1 | 296158 | 296158 | 296158 | 296158 | 15.55333302 |
| ERMS | 5 | 110497 | 110969 | 105219 | 115022 | 13.85535589 |
| FHC | 2 | 141053 | 141053 | 87056 | 195050 | 13.52930441 |
| HB | 3 | 181208 | 185167 | 127055 | 231401 | 13.54106268 |
| Hodgkins | 1 | 67071 | 67071 | 67071 | 67071 | 12.02127596 |
| MRT | 2 | 123592 | 123592 | 120378 | 126805 | 13.97823707 |
| NBL | 9 | 269160 | 269411 | 148053 | 363209 | 17.35535887 |
| OS | 4 | 324369 | 311860 | 204112 | 469644 | 15.64687998 |
| Teratoma | 2 | 196796 | 196796 | 110005 | 283588 | 14.26104037 |
| WT | 1 | 60101 | 60101 | 60101 | 60101 | 12.00787009 |

| Dx | median.width | CpG.Island.pct | CpG.Shore.pct | CpG.Shelf.pct | Open.Sea.pct |
|---|---|---|---|---|---|
| AE | 645 | 7.75313759 | 10.65315054 | 4.712691477 | 76.88102039 |
| DSRCT | 899 | 4.035683655 | 7.560829017 | 3.44647114 | 84.95701619 |
| ERMS | 586 | 9.374178102 | 10.5014486 | 5.091079853 | 75.03329345 |
| FHC | 570.5 | 13.27632115 | 10.63364559 | 4.504164344 | 71.58586892 |
| HB | 641 | 6.2632573 | 10.45647182 | 4.871019004 | 78.40925187 |
| Hodgkins | 655 | 5.371919309 | 9.573437104 | 4.544437984 | 80.5102056 |
| MRT | 548 | 15.01777853 | 12.86928578 | 4.826129723 | 67.28680596 |
| NBL | 755 | 5.127604195 | 9.332906191 | 4.463995124 | 81.07549449 |
| OS | 775.5 | 4.605250615 | 6.461949467 | 3.781598229 | 85.15120169 |
| Teratoma | 633.5 | 5.764845328 | 9.312541086 | 4.759906115 | 80.16270747 |
| WT | 478 | 7.818505516 | 12.97648958 | 6.008219497 | 73.19678541 |

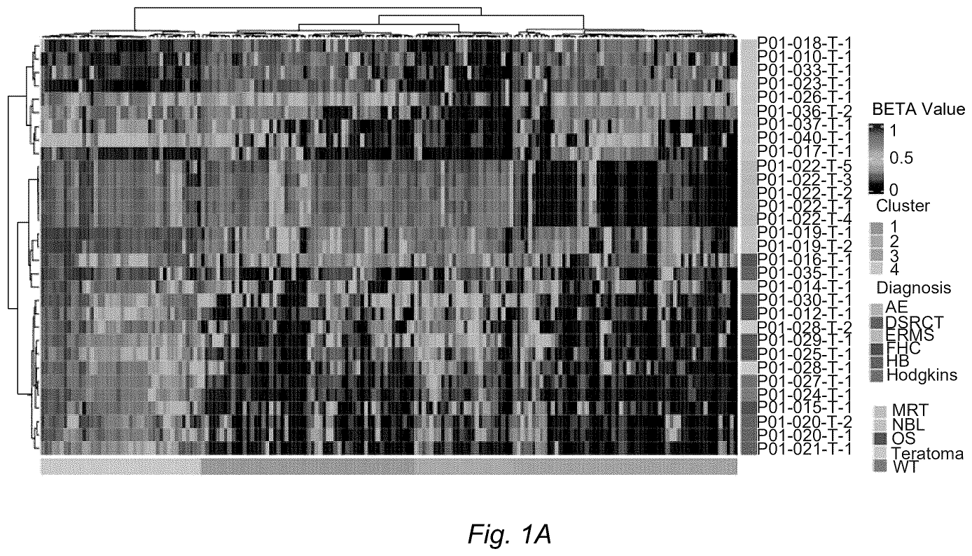

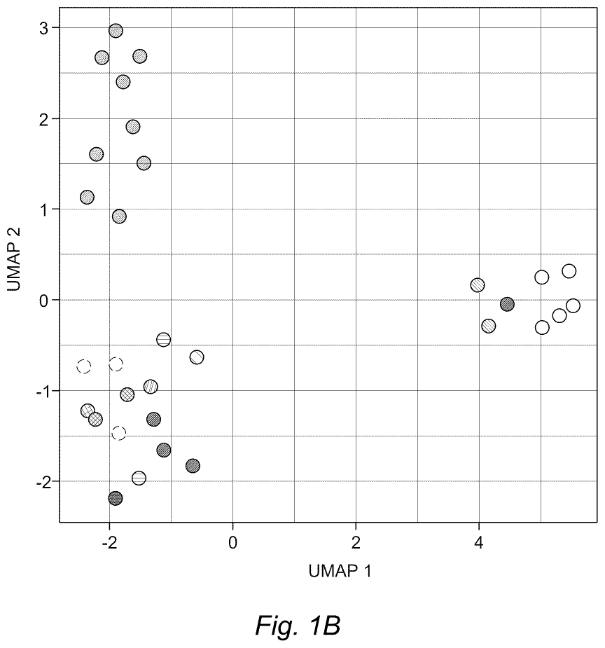

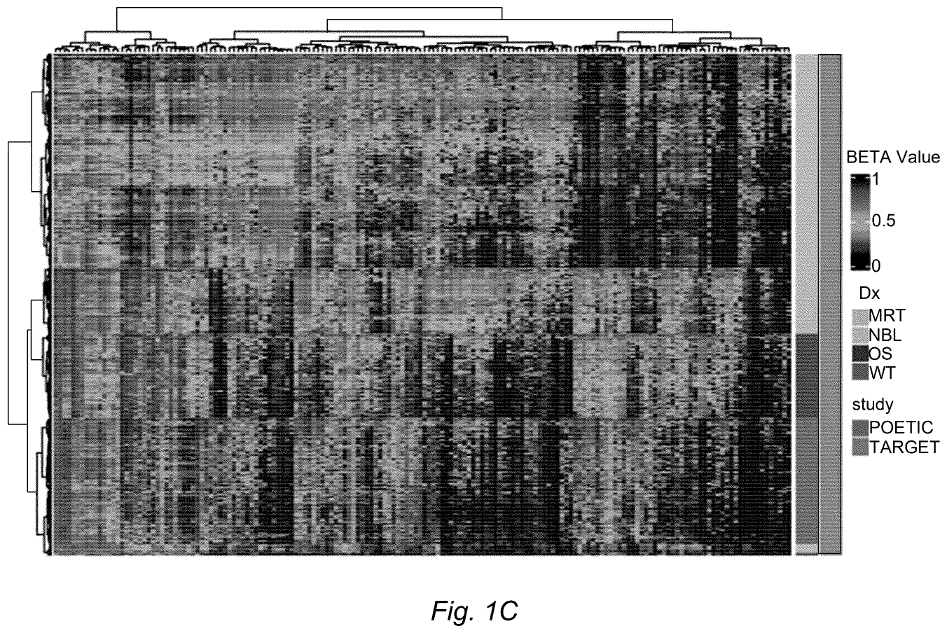

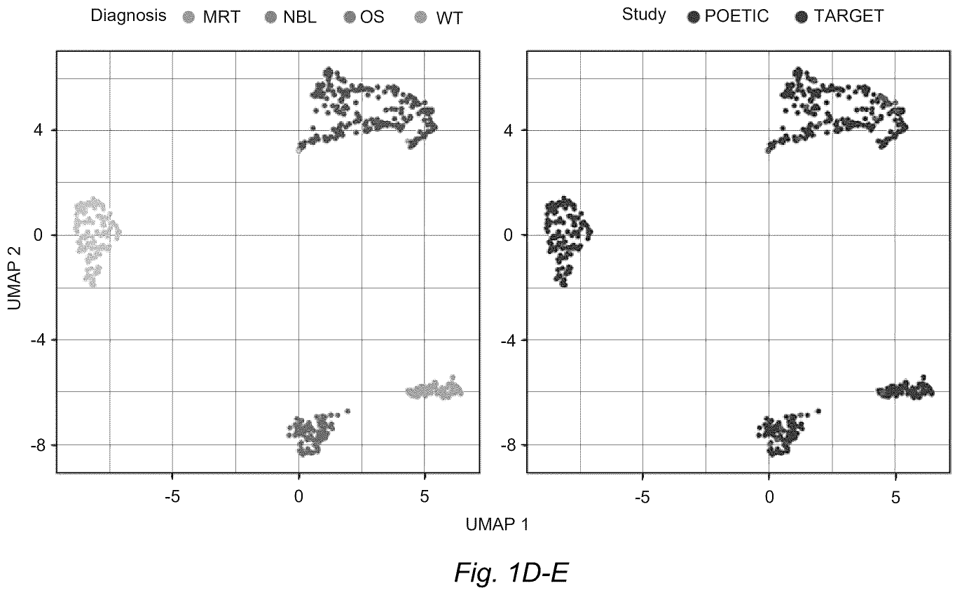

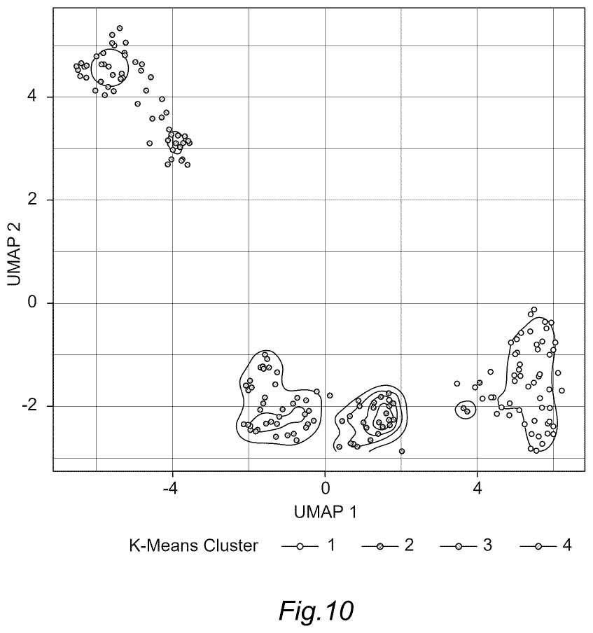

Hierarchical clustering using the 2.5% most variable statistically significant DMR calls (183 unique regions after combining overlapping regions) showed separation by tumor type and identified 4 distinct DMR subgroups (FIG. 1A-B FIG. 10). These DMRs were predominantly located at CpG islands (n=151) with the remaining DMRs located in CpG shores (n=6), shelves (n 4), and open sea (n=22). Ingenuity pathway analysis (IPA) of Cluster 1 genes found enrichment for netrin signaling and GABA receptor signaling (Table 4). Cluster 2 showed enrichment for embryonic stem cell differentiation and sonic hedgehog signaling (Table 5). Cluster 3. (FIG. 1A. Table 6. FIG. 10), which was predominately hypermethylated in neuroblastoma (NBL), was associated with ERK/MAPK signaling. Cluster 4 was hypomethylated in neuroblastoma and hypermethylated in all other tumor types. Twenty of the 32 DMR calls in this cluster were within known genes. Genes associated with this cluster included several tumor suppressors (BRCA, KANKI, ASB3, NBAT1, PIP4K2A, NFATC1, and ZNRF3) and oncogenes (HOXA3 and HOXB-AS3). IPA identified genes with DMRs in this cluster to be associated with PI3K signaling, DNA double-strand break repair, and G2/M checkpoint regulation (Table 7).

TABLE 4
IPA results from cluster 1 genes. Top canonical pathways associated with genes in
cluster 1 in FIG. 1C.

| Canonical Pathways | p-value | Ratio | Molecules |
|---|---|---|---|
| NAD biosynthesis II (from tryptophan) | 0.0001 | 0.167 | HAAO; NMNAT3 |
| nNOS Signaling in Skeletal Muscle Cells | 0.0006 | 0.0488 | CACNA1I; CACNA2D4 |
| Netrin Signaling | 0.0016 | 0.0308 | CACNA1I; CACNA2D4 |
| FcγRIIB Signaling in B Lymphocytes | 0.0021 | 0.0267 | CACNA1I; CACNA2D4 |
| CCR5 Signaling in Macrophages | 0.0034 | 0.0213 | CACNA1I; CACNA2D4 |
| GABA Receptor Signaling | 0.0034 | 0.0211 | CACNA1I; CACNA2D4 |
| GPCR-Mediated Nutrient Sensing in | 0.0047 | 0.0179 | CACNA1I; CACNA2D4 |
| Enteroendocrine Cells | | | |
| NAD Biosynthesis from 2-amino-3- | 0.0055 | 0.167 | NMNAT3 |
| carboxymuconate Semialdehyde | | | |
| Tryptophan Degradation to 2-amino-3- | 0.0055 | 0.167 | HAAO |
| carboxymuconate Semialdehyde | | | |
| NAD Biosynthesis III | 0.0055 | 0.167 | NMNAT3 |
| G Beta Gamma Signaling | 0.0056 | 0.0164 | CACNA1I; CACNA2D4 |
| Endocannabinoid Neuronal Synapse Pathway | 0.0062 | 0.0156 | CACNA1I; CACNA2D4 |
| White Adipose Tissue Browning Pathway | 0.0062 | 0.0155 | CACNA1I; CACNA2D4 |
| Thioredoxin Pathway | 0.0065 | 0.143 | TXNRD1 |
| NAD Salvage Pathway III | 0.0065 | 0.143 | NMNAT3 |
| Androgen Signaling | 0.0069 | 0.0147 | CACNA1I; CACNA2D4 |
| Type II Diabetes Mellitus Signaling | 0.0074 | 0.0141 | CACNA1I; CACNA2D4 |
| Corticotropin Releasing Hormone Signaling | 0.0078 | 0.0138 | CACNA1I; CACNA2D4 |
| PKCθ Signaling in T Lymphocytes | 0.0089 | 0.0129 | CACNA1I; CACNA2D4 |
| Gustation Pathway | 0.0089 | 0.0129 | CACNA1I; CACNA2D4 |
| GNRH Signaling | 0.011 | 0.0116 | CACNA1I; CACNA2D4 |
| Synaptic Long Term Depression | 0.0129 | 0.0106 | CACNA1I; CACNA2D4 |
| Calcium Signaling | 0.0151 | 0.00971 | CACNA1I; CACNA2D4 |
| Role of NFAT in Cardiac Hypertrophy | 0.0162 | 0.00935 | CACNA1I; CACNA2D4 |
| CREB Signaling in Neurons | 0.0166 | 0.00503 | CACNA1I; CACNA2D4; |
| | | | OXTR |
| Tryptophan Degradation III (Eukaryotic) | 0.0209 | 0.0435 | HAAO |
| Opioid Signaling Pathway | 0.0214 | 0.0081 | CACNA1I; CACNA2D4 |
| Vitamin-C Transport | 0.0219 | 0.0417 | TXNRD1 |
| NAD Salvage Pathway II | 0.0234 | 0.0385 | NMNAT3 |

TABLE 5
IPA results from cluster 2 genes. Top canonical pathways associated
with genes in cluster 2.

| Canonical Pathways | p-value | Ratio | Molecules |
|---|---|---|---|
| Embryonic Stem Cell Differentiation into Cardiac | 0.0079 | 0.1 | GATA4 |
| Lineages | | | |
| Cardiomyocyte Differentiation via BMP Receptors | 0.0158 | 0.05 | GATA4 |
| Sonic Hedgehog Signaling | 0.0224 | 0.0345 | GLI3 |
| Transcriptional Regulatory Network in Embryonic Stem | 0.0417 | 0.0185 | GATA4 |
| Cells | | | |

TABLE 6
IPA results from cluster 3 genes. Top canonical pathways
associated with genes in cluster 3.

| Canonical Pathways | p-value | Ratio | Molecules |
|---|---|---|---|
| Gas Signaling | 0.0145 | 0.0187 | PTGER4; SRC |
| Glycine Betaine Degradation | 0.017 | 0.1 | DMGDH |
| Ephrin Receptor Signaling | 0.0417 | 0.0106 | EFNA2; SRC |
| ERK/MAPK Signaling | 0.0468 | 0.0099 | SRC; VRK2 |

TABLE 7
IPA results from cluster 4 genes. Top canonical pathways associated
with genes in cluster 4.

| | p- | | |
|---|---|---|---|
| Canonical Pathways | value | Ratio | Molecules |
| S-methyl-5′-thioadenosine Degradation II | 0.0023 | 0.333 | MTAP |
| PI3K Signaling in B Lymphocytes | 0.0052 | 0.0145 | IRS2; NFATC1 |
| Glycogen Degradation II | 0.0093 | 0.0833 | MTAP |
| DNA Double-Strand Break Repair by Homologous | 0.011 | 0.0714 | BRCA1 |
| Recombination | | | |
| Glycogen Degradation III | 0.011 | 0.0714 | MTAP |
| Glutaryl-CoA Degradation | 0.0126 | 0.0625 | CDYL |
| GADD45 Signaling | 0.0148 | 0.0526 | BRCA1 |
| DNA damage-induced 14-3-3σ Signaling | 0.0148 | 0.0526 | BRCA1 |
| Tryptophan Degradation III (Eukaryotic) | 0.0182 | 0.0435 | CDYL |
| D-myo-inositol (1,4,5)-Trisphosphate Biosynthesis | 0.0195 | 0.04 | PIP4K2A |
| IL-9 Signaling | 0.0257 | 0.0303 | IRS2 |
| Role of JAK2 in Hormone-like Cytokine Signaling | 0.0263 | 0.0294 | IRS2 |
| April Mediated Signaling | 0.0309 | 0.025 | NFATC1 |
| B Cell Activating Factor Signaling | 0.0316 | 0.0244 | NFATC1 |
| Role of Oct4 in Mammalian Embryonic Stem Cell | 0.0355 | 0.0217 | BRCA1 |
| Pluripotency | | | |
| Cell Cycle: G2/M DNA Damage Checkpoint | 0.038 | 0.0204 | BRCA1 |
| Regulation Amyloid Processing | 0.0389 | 0.02 | CDK5R1 |
| Transcriptional Regulatory Network in Embryonic | 0.0417 | 0.0185 | CDYL |
| Stem Cells | | | |
| Role of CHK Proteins in Cell Cycle Checkpoint | 0.0437 | 0.0175 | BRCA1 |
| Control | | | |
| Wnt/Ca+ pathway | 0.0479 | 0.0161 | NFATC1 |
| Thrombopoietin Signaling | 0.049 | 0.0159 | IRS2 |

Methylation beta values were also extracted from 518 TARGET samples (Table 8) across 166 of the 183 highly variable DMRs which overlapped at least one probe on the HM450 methylation array. Analysis of TARGET data also demonstrated strong separation by tumor type (FIG. 1C-D). POETIC samples clustered with TARGET samples according to tumor type as seen by hierarchical clustering and UMAP analyses (FIG. 1E). It is also important to note that TARGET samples were predominantly collected from the primary tumors (Table 8), while POETIC cases were all collected from patients with recurrent metastatic disease, indicating that methylation profiles of recurrent tumors are more similar to primary tumors then they are different. The co-clustering of POETIC recurrent samples with TARGET samples was also observed when each tumor type was analyzed separately (data not shown).

TABLE 8
TARGET sample summary. Table shows the number of samples
for each cancer type accessed from the TARGET database.

| short_dx | sample_type | n |
|---|---|---|
| MRT | Primary Tumor | 68 |
| NBL | Primary Tumor | 214 |
| NBL | Recurrence | 9 |
| OS | Primary Tumor | 104 |
| WT | Primary Tumor | 125 |
| WT | Recurrence | 6 |

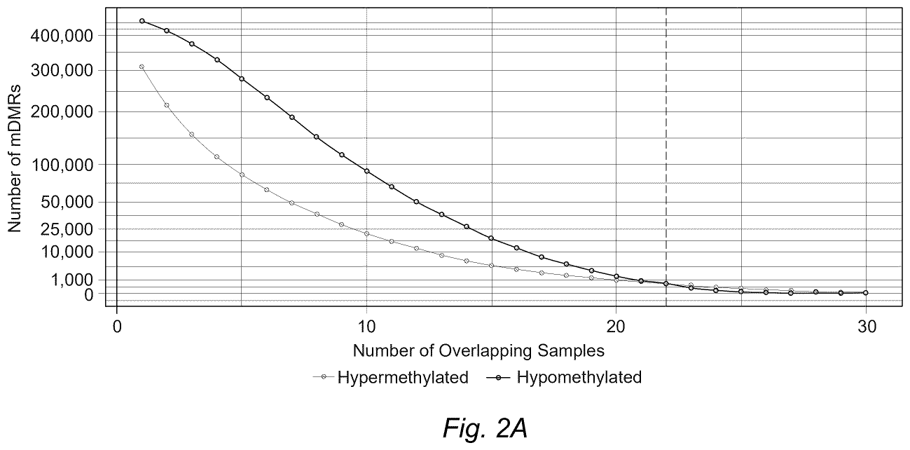

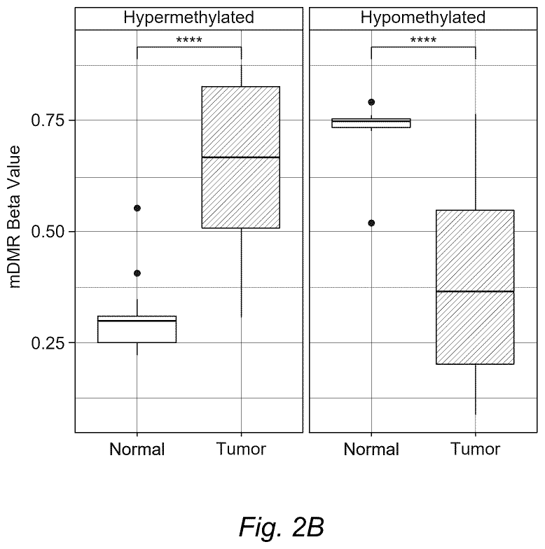

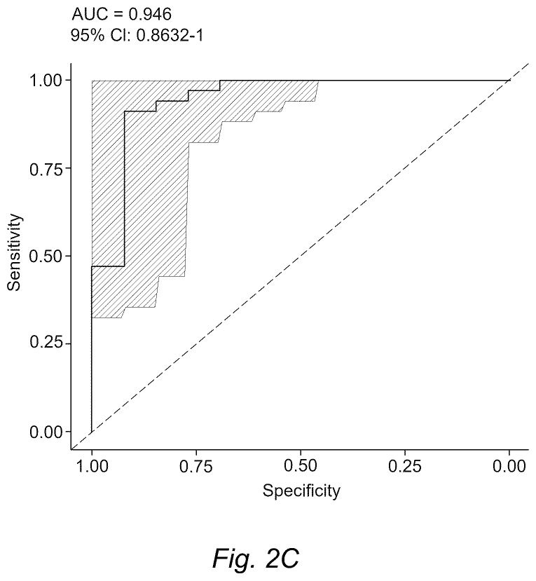

DNA methylation alterations shared across tumor types. We identified sets of minimally differentially methylated regions (mDMRs), as subregions of each DMR that were shared (in the same direction; hypo- or hyper-methylated) across multiple samples (and cancer types). Briefly, each CpG site was scored based on the number of samples with a DMR call which included that CpG (separately for hypomethylated and hypermethylated DMR calls). Clusters of adjacent CpG sites, each having a DMR present in at least N samples, were merged to create contiguous mDMR regions. The number of shared mDMRs decreased as N increased, for both hypo and hypermethylated regions (FIG. 2A), but it was possible to identify a set of mDMRs shared between at least 70% (n=22 of 31) of samples. This subset of mDMRs included 402 hypomethylated regions, with a median width of 276 bp, and 503 hypermethylated regions (total=905, FIG. 2A), with a median width of 230 bp. Mean beta values across hypomethylated mDMRs were 0.406 in tumor tissue compared with 0.732 in normal tissue. Beta values in hypermethylated mDMRs were 0.643 in tumor tissue compared with 0.308 in normal tissues (FIG. 2B).

A random forest classifier was built, based on methylation patterns of the mDMR set, to determine the utility of using the data for tumor detection. The cross-validated receiver operating characteristic (ROC) of methylation in mDMRs had an area under the curve (AUC) of 0.95, indicating that the mDMRs were capable of differentiating tumor from nonnal (FIG. 2C).

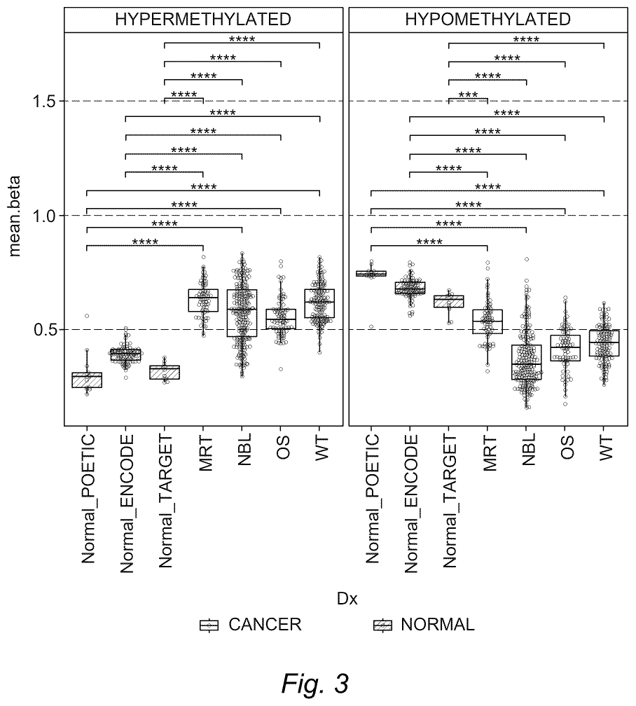

To further validate the mDMR signature we analyzed 518 samples from the TARGET database (Table 8) for Wilms tumor (WT), MRT, osteosarcoma (OS), and NBL samples. We also analyzed 90 normal tissue samples from multiple tissue types from ‘The Encyclopedia of DNA Elements’ (ENCODE) (Table 9). Both of these datasets were analyzed using Infinium methylation HM450 and EPIC arrays (Illumina).We were able to assess mean beta value at 344 hypermethylated and 71 hypomethylated mDMRs which overlapped at least 1 probe on the HM450 and EPIC bead chips. We found that methylation patterns of mDMRs in TARGET data resembled POETIC WGBS data and were hyper or hypomethylated in tumors compared to normal controls (FIG. 3. Wilcoxon p-value<0.001 for all comparisons). Taken together, these results indicate that the mDMRs identified are generalizable across multiple pediatric cancer types.

TABLE 9
ENCODE sample summary. Table shows the number of samples
for each tissue type accessed from the ENCODE database.

| tissue type | n |
|---|---|
| Adipose tissue | 8 |
| Adrenal gland tissue | 3 |
| colon tissue | 8 |
| gastrointestinal tissue | 18 |
| liver tissue | 1 |
| lung tissue | 4 |
| muscle tissue | 4 |
| nervous tissue | 4 |
| pancreatic tissue | 4 |
| reproductive tissue | 13 |
| skin tissue | 8 |
| spleen tissue | 3 |
| Thyroid gland tissue | 4 |
| vascular tissue | 8 |
| Total | 90 |

Hypomethylated mDMRs were located across the genome, with 152 mDMRs within known genes, 30 within 2000 bp of a gene transcriptional start site, and 220 in intergenic regions. A majority of these regions were located in open sea (n=328) with 16 regions in CpG islands, 39 in CpG shores, and 18 in CpG shelves. IPA analysis showed genes associated with hypomethylated mDMRs were involved in several immune signaling pathways—including natural killer cell signaling, WNT/β-catenin signaling, and TREM1 signaling (Table 10).

TABLE 10
IPA results from hypomethylated mDMRs. Top canonical pathways associated with
genes in hypomethylated mDMRs.

| | p- | | |
|---|---|---|---|
| Canonical Pathways | value | Ratio | Molecules |
| GABA Receptor Signaling | 0 | 0.0458 | CACNA1A; CACNG6; GNA12; GNG7; |
| | | | ITPR2; KCNQ3 |
| RHOGDI Signaling | 0.0001 | 0.0326 | ARHGAP8/PRR5-ARHGAP8; CDH4; |
| | | | GNA12; GNG7; ITGAD; ITGAX; MYH14 |
| Androgen Signaling | 0.0001 | 0.0355 | CACNA1A; CACNG6; GNA12; GNG7; |
| | | | ITPR2; TAF2 |
| GPCR-Mediated Nutrient Sensing in | 0.0002 | 0.0431 | CACNA1A; CACNG6; GNG7; ITPR2; |
| Enteroendocrine Cells | | | PYY |
| Phagosome Formation | 0.0003 | 0.016 | ADGRB1; ADGRE3; AP1M2; FCER2; |
| | | | HRH2; ITGAD; ITGAX; ITPR2; MYH14; |
| | | | SSTR5; VAV1 |
| G Beta Gamma Signaling | 0.0003 | 0.0388 | CACNA1A; CACNG6; GNA12; GNG7; |
| | | | ITPR2 |
| CREB Signaling in Neurons | 0.0004 | 0.0166 | ADGRB1; ADGRE3; CACNA1A; |
| | | | CACNG6; GNA12; GNG7; HRH2; ITPR2; |
| | | | NTRK2; SSTR5 |
| Cellular Effects of Sildenafil (Viagra) | 0.0005 | 0.0336 | CACNA1A; CACNG6; ITPR2; KCNQ3; |
| | | | MYH14 |
| Crosstalk between Dendritic Cells and | 0.0007 | 0.044 | KIR2DL1/KIR2DL3; KIR3DL1; |
| Natural Killer Cells | | | KIR3DL2; KIR3DL3 |
| Cardiac β-adrenergic Signaling | 0.0011 | 0.0287 | CACNA1A; CACNG6; GNA12; |
| | | | GNG7; PDE6B |
| Signaling by Rho Family GTPases | 0.0013 | 0.0224 | CDH4; GNA12; GNG7; ITGAD; |
| | | | ITGAX; SEPTIN14 |
| nNOS Signaling in Skeletal Muscle | 0.0013 | 0.0625 | CACNA1A; CACNG6; ITPR2 |
| Cells | | | |
| Natural Killer Cell Signaling | 0.002 | 0.0251 | KIR2DL1/KIR2DL3; KIR3DL1; |
| | | | KIR3DL2; KIR3DL3; VAV1 |
| Cardiac Hypertrophy Signaling | 0.0028 | 0.0149 | CACNA1A; CACNG6; GNA12; GNG7; |
| (Enhanced) | | | ITGAD; ITGAX; ITPR2; PDE6B |
| Ephrin B Signaling | 0.0041 | 0.0417 | GNA12; GNG7; VAV1 |
| Netrin Signaling | 0.0041 | 0.0417 | CACNA1A; CACNG6; ITPR2 |
| Dilated Cardiomyopathy Signaling | 0.0042 | 0.0274 | CACNA1A; CACNG6; ITPR2; MYH14 |
| Pathway | | | |

Hypomethylated mDMRs were more likely to be associated with genes than hypomethylated mDMRs; 316 of 503 regions were within known genes (63% compared to 38 in hypomethylated regions). Most hypermethylated mDMRs were located in CpG islands (n=387) with the remaining mDMRs located in CpG shores (n=6), shelves (n=4), and open sea (n=22). Hypermethylated mDMRs were disproportionately associated with protocadherin genes PCDHGA8, PCDHGA1, PCDHA1, and PCDHA9. IPA found that genes associated with the selected mDMR were significantly associated with the regulation of epithelial-mesenchymal transition (EM), NANOG signaling TGF-signaling, and TREM1 signaling (Table 11). Taken together, these results show that the selected mDMRs may represent a pan-pediatric cancer signature, which is associated with broad-spanning cancer-specific pathways.

TABLE 11
IPA results from hypermethylated mDMRs. Top canonical pathways associated with
genes in hypermethylated mDMRs.

| Canonical Pathways | p-value | Ratio | Molecules |
|---|---|---|---|
| IL-12 Signaling and Production | 0.0025 | 0.0441 | IL12RB2; MAPK13; MST1R; PIK3CD; |
| in | | | PIK3R4; RXRA |
| Macrophages | | | |
| Non-Small Cell Lung Cancer | 0.0025 | 0.0532 | CDK6; PIK3CD; PIK3R4; RASSF1; RXRA |
| Signaling | | | |
| CREB Signaling in Neurons | 0.003 | 0.0233 | ADGRL4; ADRA1A; ADRA2A; ADRA2B; |
| | | | CACNA1I; CACNA2D4; GPR62; GRID1; |
| | | | LPAR5; NPFFR1; NTRK1; PDGFRA; PIK3CD; |
| | | | PIK3R4 |
| Chronic Myeloid Leukemia | 0.0044 | 0.0467 | CDK6; CTBP2; PIK3CD; PIK3R4; STAT5A |
| Signaling | | | |
| Cardiac Hypertrophy Signaling | 0.0045 | 0.031 | ADRA1A; ADRA2A; ADRA2B; |
| | | | CACNA2D4; MAPK13; PIK3CD; PIK3R4; |
| | | | RHOU |
| IL-9 Signaling | 0.005 | 0.0857 | PIK3CD; PIK3R4; STAT5A |
| Role of Tissue Factor in Cancer | 0.0062 | 0.0431 | FGR; MAPK13; PIK3CD; PIK3R4; STAT5A |
| Human Embryonic Stem Cell | 0.0065 | 0.0361 | LEFTY1; LEFTY2; NTRK1; PDGFRA; |
| Pluripotency | | | PIK3CD; PIK3R4 |
| IL-7 Signaling Pathway | 0.0078 | 0.0513 | MAPK13; PIK3CD; PIK3R4; STAT5A |
| IL-15 Production | 0.0078 | 0.0407 | FGR; MST1R; NTRK1; PDGFRA; ROR2 |
| FLT3 Signaling in | 0.0083 | 0.05 | MAPK13; PIK3CD; PIK3R4; STAT5A |
| Hematopoietic | | | |
| Progenitor Cells | | | |
| Reelin Signaling in Neurons | 0.0087 | 0.0397 | ARHGEF4; FGR; MAPK8IP2; PIK3CD; |
| | | | PIK3R4 |
| FGF Signaling | 0.01 | 0.0476 | FGF8; MAPK13; PIK3CD; PIK3R4 |
| FcγRIIB Signaling in B | 0.0105 | 0.0471 | CACNA1I; CACNA2D4; PIK3CD; PIK3R4 |
| Lymphocytes | | | |
| IL-23 Signaling Pathway | 0.0107 | 0.0652 | IL12RB2; PIK3CD; PIK3R4 |
| AMPK Signaling | 0.011 | 0.0289 | ADRA1A; ADRA2A; ADRA2B; CHRNE; |
| | | | MAPK13; PIK3CD; PIK3R4 |
| White Adipose Tissue Browning | 0.0117 | 0.0368 | CACNA1I; CACNA2D4; CTBP2; MAPK13; |
| Pathway | | | RXRA |
| Regulation Of The Epithelial | 0.0126 | 0.0312 | FGF8; MAPK13; MEST; PDGFRA; |
| Mesenchymal | | | PIK3CD; PIK3R4 |
| Transition By Growth Factors | | | |
| Pathway | | | |
| Xenobiotic Metabolism General | 0.0145 | 0.035 | MAPK13; PIK3CD; PIK3R4; RXRA; |
| Signaling Pathway | | | SLC51A |
| UVB-Induced MAPK Signaling | 0.0151 | 0.0577 | MAPK13; PIK3CD; PIK3R4 |
| Small Cell Lung Cancer | 0.0155 | 0.0417 | CDK6; PIK3CD; PIK3R4; RXRA |
| Signaling | | | |
| EGF Signaling | 0.0174 | 0.0545 | MAPK13; PIK3CD; PIK3R4 |
| Insulin Secretion Signaling | 0.0182 | 0.0261 | CACNA1I; CACNA2D4; FGR; MAPK13; |
| Pathway | | | PIK3CD; PIK3R4; STAT5A |
| Role of IL-17A in Arthritis | 0.0191 | 0.0526 | MAPK13; PIK3CD; PIK3R4 |
| MSP-RON Signaling Pathway | 0.02 | 0.0517 | MST1R; PIK3CD; PIK3R4 |
| eNOS Signaling | 0.0219 | 0.0314 | AQP2; CHRNE; LPAR5; PIK3CD; |
| | | | PIK3R4 |
| IL-2 Signaling | 0.0229 | 0.0492 | PIK3CD; PIK3R4; STAT5A |
| IL-22 Signaling | 0.0234 | 0.0833 | MAPK13; STAT5A |
| Thrombopoietin Signaling | 0.0251 | 0.0476 | PIK3CD; PIK3R4; STAT5A |
| Role of JAK family kinases in | 0.0257 | 0.08 | MAPK13; STAT5A |
| IL-6-type | | | |
| Cytokine Signaling | | | |
| ERB2-ERBB3 Signaling | 0.0269 | 0.0462 | PIK3CD: PIK3R4; STAT5A |
| Amyotrophic Lateral Sclerosis | 0.0282 | 0.0348 | CACNA2D4; GRID1; PIK3CD; PIK3R4 |
| Signaling | | | |
| Glioblastoma Multiforme | 0.0282 | 0.0292 | CDK6; PDGFRA; PIK3CD; PIK3R4; RHOU |
| Signaling | | | |
| Superpathway of Inositol | 0.0288 | 0.0259 | ACP5; ITPKA; PIK3CD; PIK3R4; PIP5K1C; |
| Phosphate | | | PPFIA3 |
| Compounds | | | |
| CD40 Signaling | 0.0295 | 0.0448 | MAPK13; PIK3CD; PIK3R4 |
| IL-17A Signaling in Airway | 0.0295 | 0.0448 | MAPK13; PIK3CD; PIK3R4 |
| Cells | | | |
| S-methyl-5′-thioadenosine | 0.0295 | 0.333 | MTAP |
| Degradation II | | | |
| Sphingosine-1-phosphate | 0.0302 | 0.0339 | PDGFRA; PIK3CD; PIK3R4; RHOU |
| Signaling | | | |
| Role of JAK1 and JAK3 in γc | 0.0316 | 0.0435 | PIK3CD; PIK3R4; STAT5A |
| Cytokine | | | |
| Signaling | | | |
| Role of p14/p19ARF in Tumor | 0.0339 | 0.069 | PIK3CD; PIK3R4 |
| Suppression | | | |
| Growth Hormone Signaling | 0.0339 | 0.0423 | PIK3CD; PIK3R4; STAT5A |
| Glioma Signaling | 0.0355 | 0.0323 | CDK6; PDGFRA; PIK3CD; PIK3R4 |
| Breast Cancer Regulation by | 0.0355 | 0.0186 | ADGRL4; ADRA1A; ADRA2A; ADRA2B; |
| Stathmin1 | | | ARHGEF4; CDK6; GPR62; LPAR5; NPFFR1; |
| | | | PIK3CD; PIK3R4 |
| Glioma Invasiveness Signaling | 0.0363 | 0.0411 | PIK3CD; PIK3R4; RHOU |
| Leptin Signaling in Obesity | 0.038 | 0.0405 | PIK3CD; PIK3R4; POMC |
| Neurotrophin/TRK Signaling | 0.0407 | 0.0395 | NTRK1; PIK3CD; PIK3R4 |
| Angiopoietin Signaling | 0.0417 | 0.039 | PIK3CD; PIK3R4; STAT5A |
| Sperm Motility | 0.0427 | 0.0236 | CACNA1I; FGR; MST1R; NTRK1; PDGFRA; |
| | | | ROR2 |
| Phagosome Formation | 0.0437 | 0.0174 | ADGRL4; ADRA1A; ADRA2A; ADRA2B; |
| | | | ELMO3; |
| | | | FGR; GPR62; LPAR5; NPFFR1; PIK3CD; |
| | | | PIK3R4; |
| | | | PIP5K1C |
| Gα12/13 Signaling | 0.0447 | 0.0301 | CDH23; LPAR5; PIK3CD; PIK3R4 |
| Clathrin-mediated Endocytosis | 0.0447 | 0.0259 | DNM1; FGF8; PIK3CD; PIK3R4; PIP5K1C |
| Signaling | | | |
| IL-3 Signaling | 0.0447 | 0.038 | PIK3CD; PIK3R4; STAT5A |
| Thyroid Cancer Signaling | 0.0447 | 0.038 | NTRK1; PIK3CD; PIK3R4 |
| Estrogen-Dependent Breast | 0.0447 | 0.038 | PIK3CD; PIK3R4; STAT5A |
| Cancer Signaling | | | |
| STAT3 Pathway | 0.0468 | 0.0296 | IL12RB2; MAPK13; NTRK1; PDGFRA |
| Th2 Pathway | 0.0479 | 0.0292 | IL12RB2; PIK3CD; PIK3R4; STAT5A |
| JAK/STAT Signaling | 0.049 | 0.0366 | PIK3CD; PIK3R4; STAT5A |
| RAC Signaling | 0.049 | 0.029 | ANK1; PIK3CD; PIK3R4; PIP5KIC |

TABLE 12
TCGA sample summary. Table shows the number of samples for each cancer
type (tumor and adjacent normal) accessed from the TCGA database.

| Tissue | | | | | | | | | | | | | | | |
|---|---|---|---|---|---|---|---|---|---|---|---|---|---|---|---|
| Type | BLCA | BRCA | COAD | ESCA | HNSC | KIRC | KIRP | LIHC | LUAD | LUSC | PAAD | PRAD | THCA | UCEC | Total |
| Cancer | 419 | 796 | 315 | 186 | 530 | 325 | 276 | 380 | 475 | 370 | 185 | 503 | 515 | 439 | 5714 |
| Normal | 21 | 96 | 38 | 16 | 50 | 160 | 45 | 50 | 32 | 42 | 10 | 50 | 56 | 46 | 712 |
| Total | 440 | 892 | 353 | 202 | 580 | 485 | 321 | 430 | 507 | 412 | 195 | 553 | 571 | 485 | 6426 |

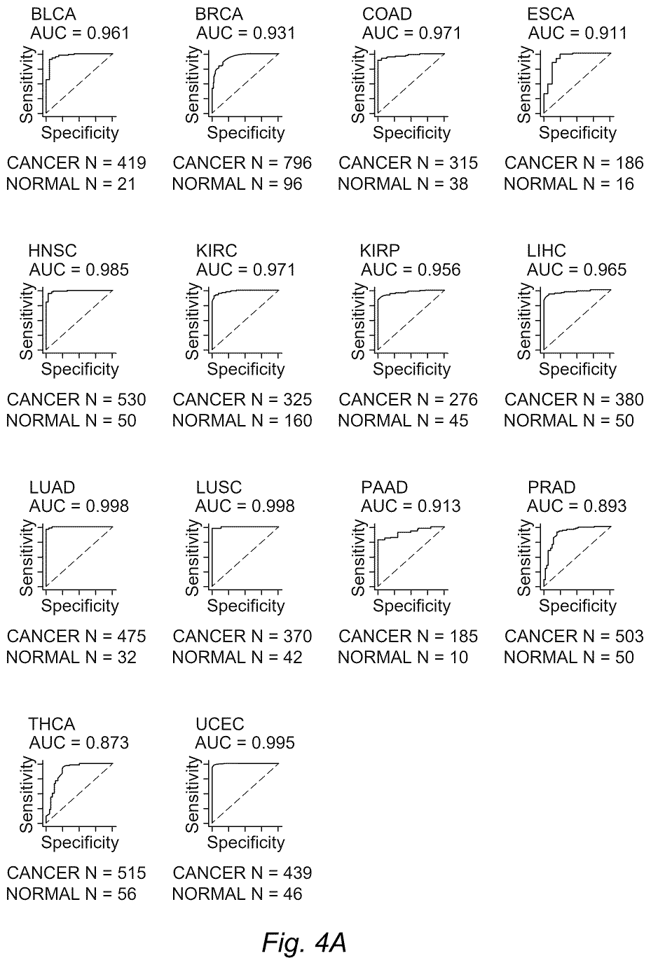

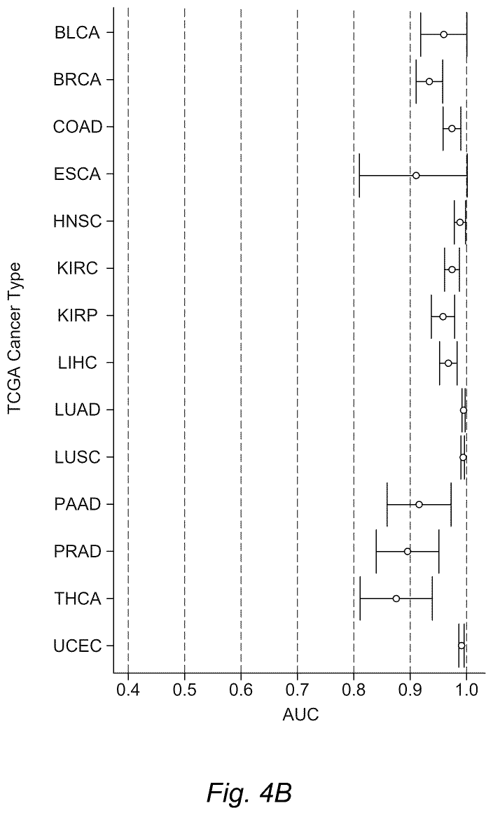

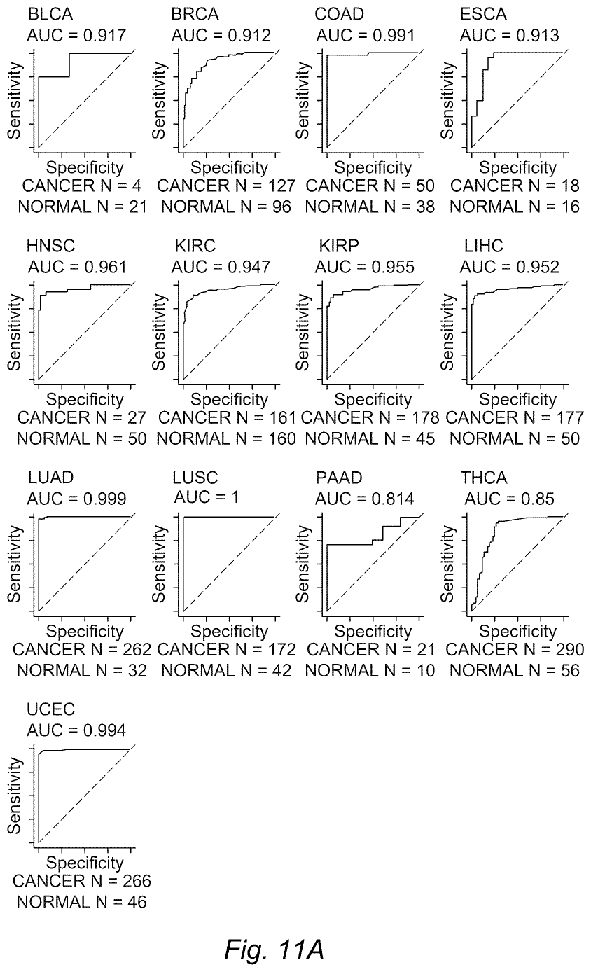

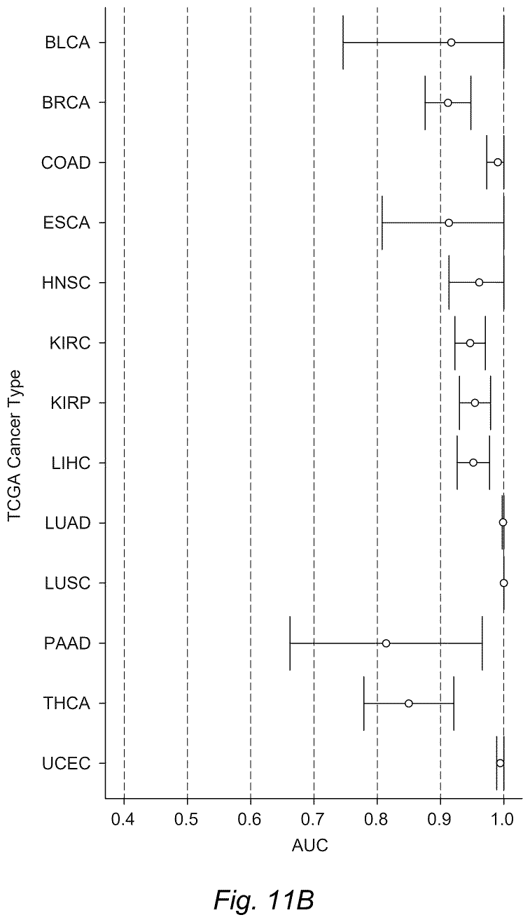

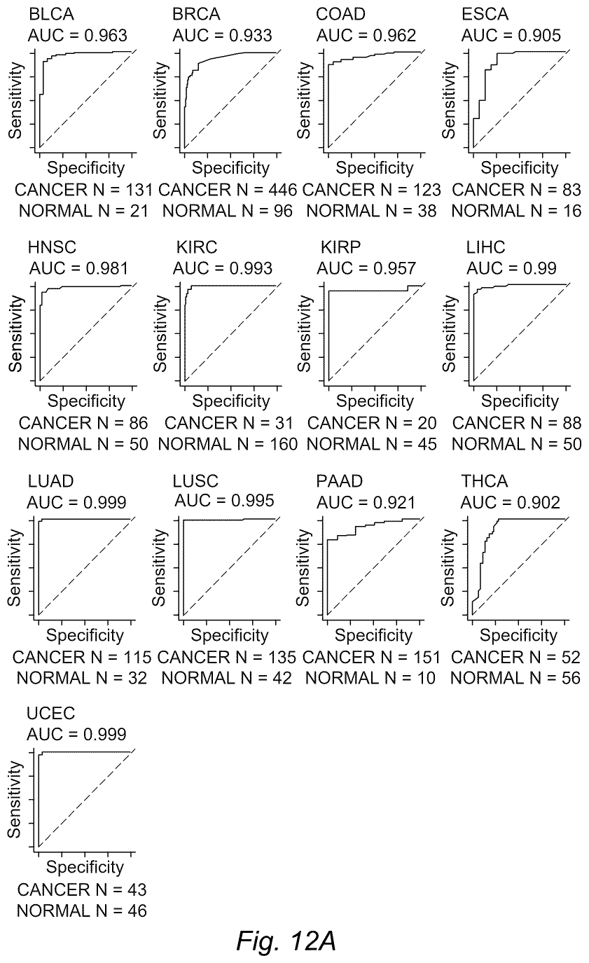

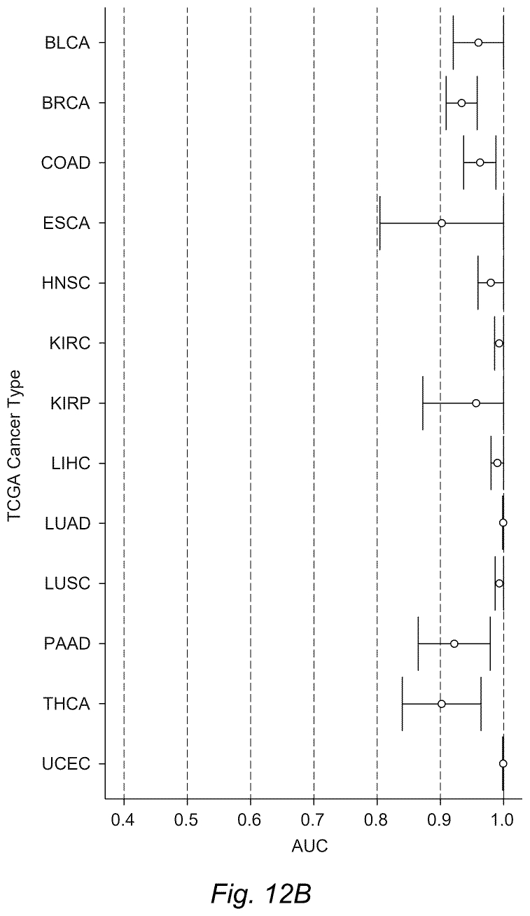

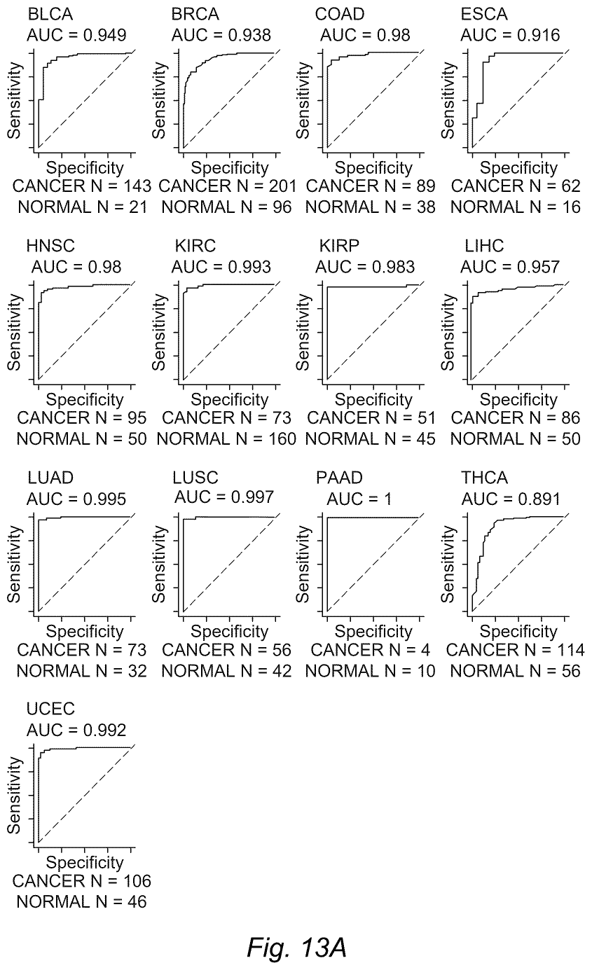

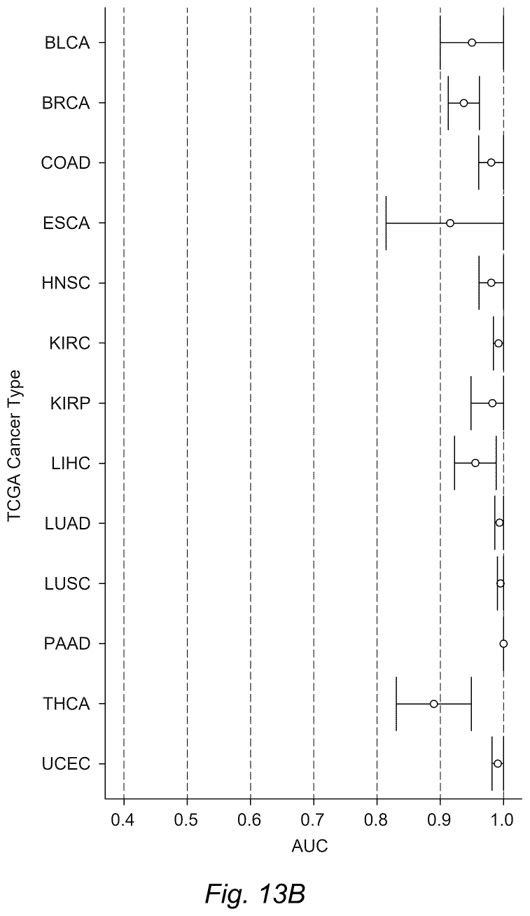

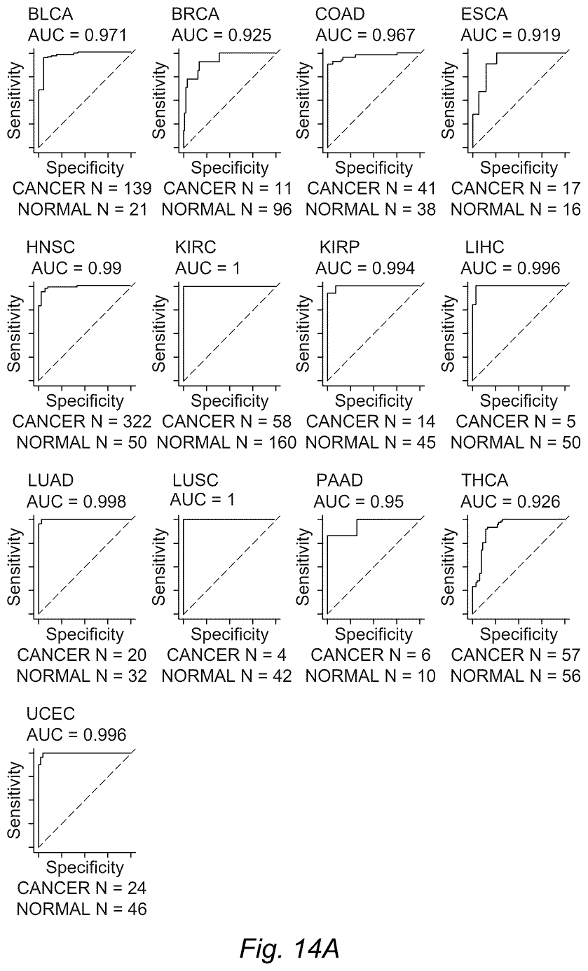

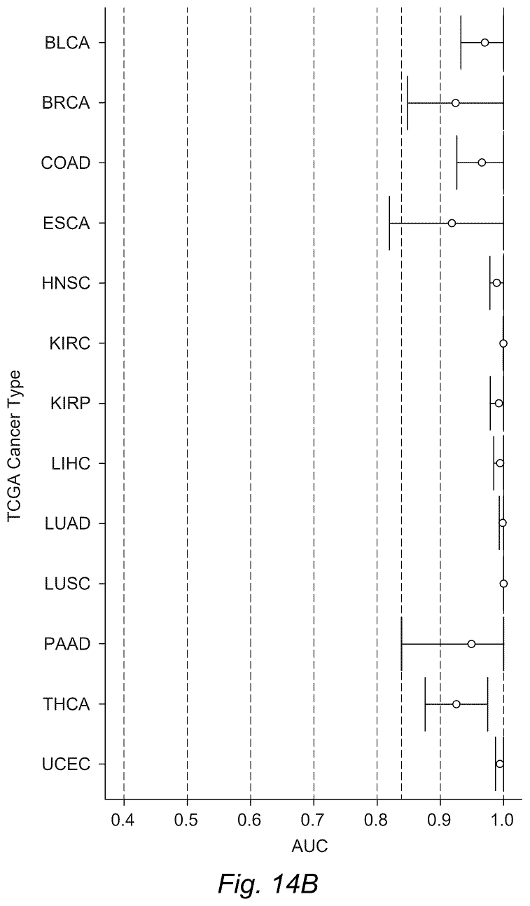

mDMRs in pediatric cancer are also detected in adult cancers. To determine the generalizability of the 905 mDMRs across a broad set of adult tumor types, we used HM450 data from TCGA to assess the tumor/nonnal classifier. Four hundred and twenty two of 905 mDMRs overlapped with at least one probe on the HM450 array, and we built a separate random forest classifier based on this reduced set of regions. ROC curves (and AUCs) were calculated for 14 different adult solid tumors (6426 samples, Table 14) (FIG. 4A-B). Our model achieved an average AUC of 0.95 indicating that the reduced CpG mDMR set was able to distinguish tumor from normal in samples obtained from TCGA. All but 2 cancer types (THCA—thyroid cancer, and PRAD—prostate adenocarcinoma) achieved an AUC>0.9 (FIG. 4C). Performance was consistent across all TCGA tumor stages (FIG. 11-14). This indicates that the panel of CpG mDMRs identified could potentially serve as a pan-cancer methylation biomarker applied to both pediatric and adult cancers.

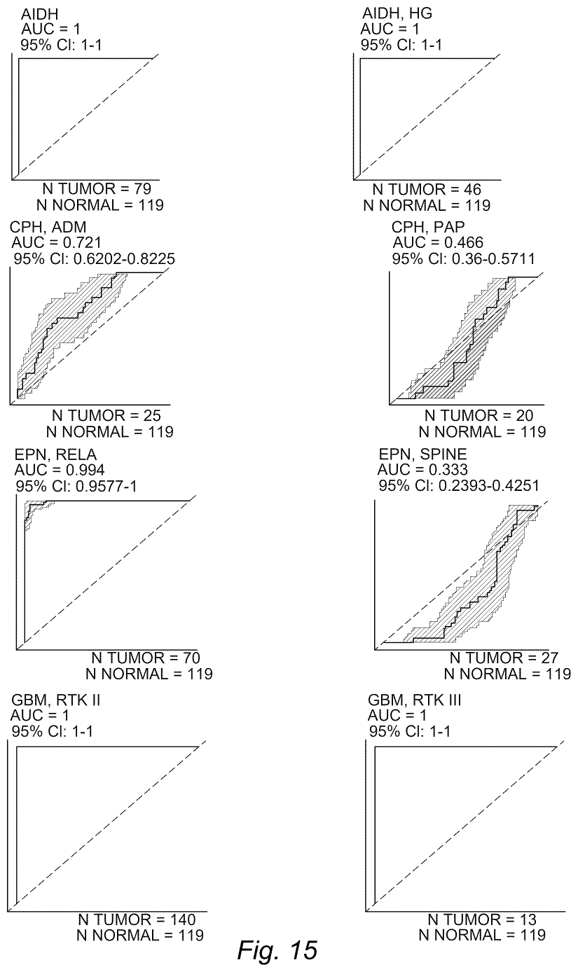

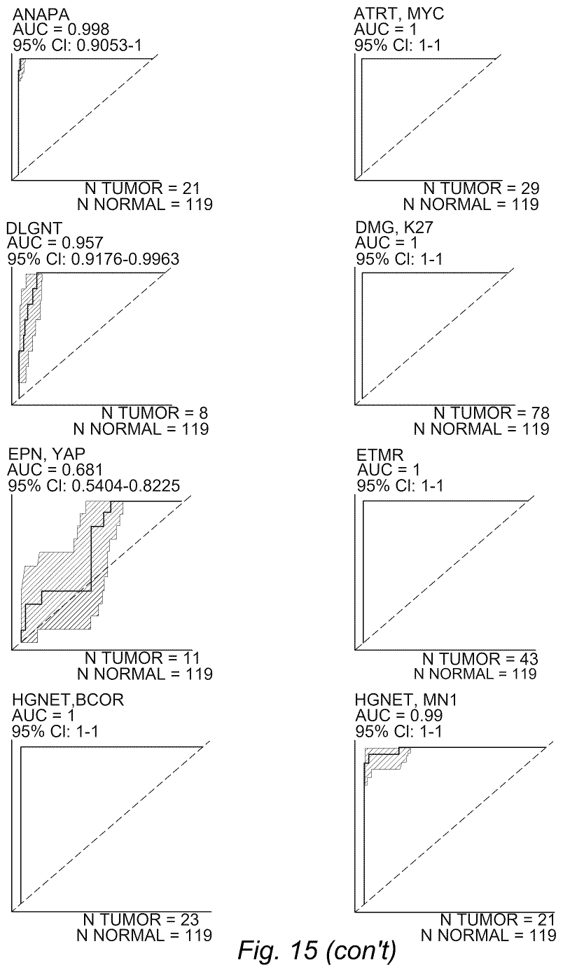

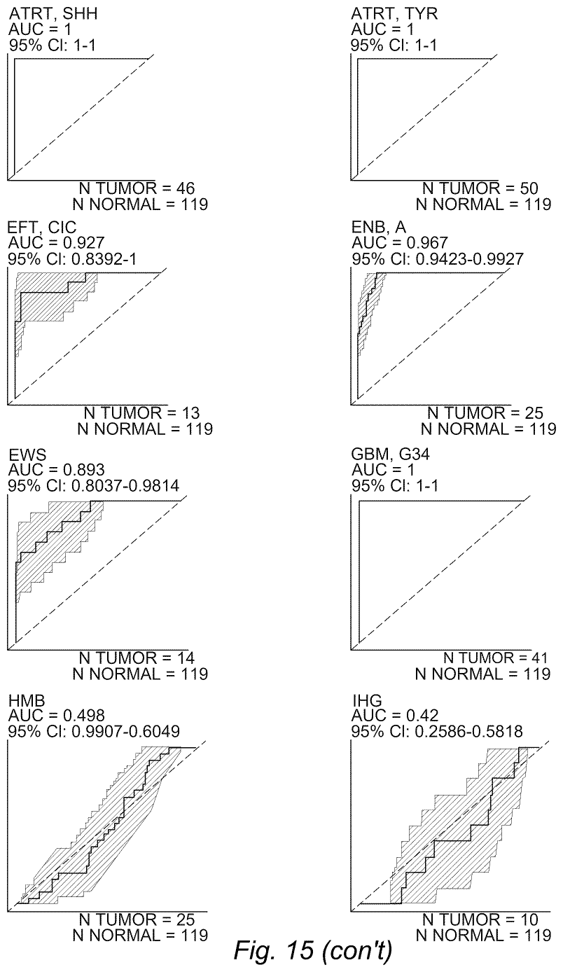

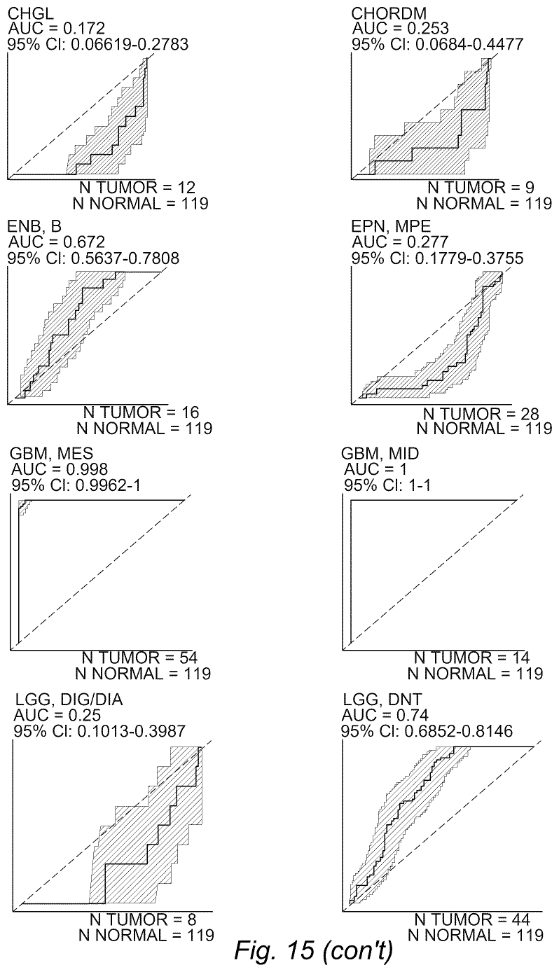

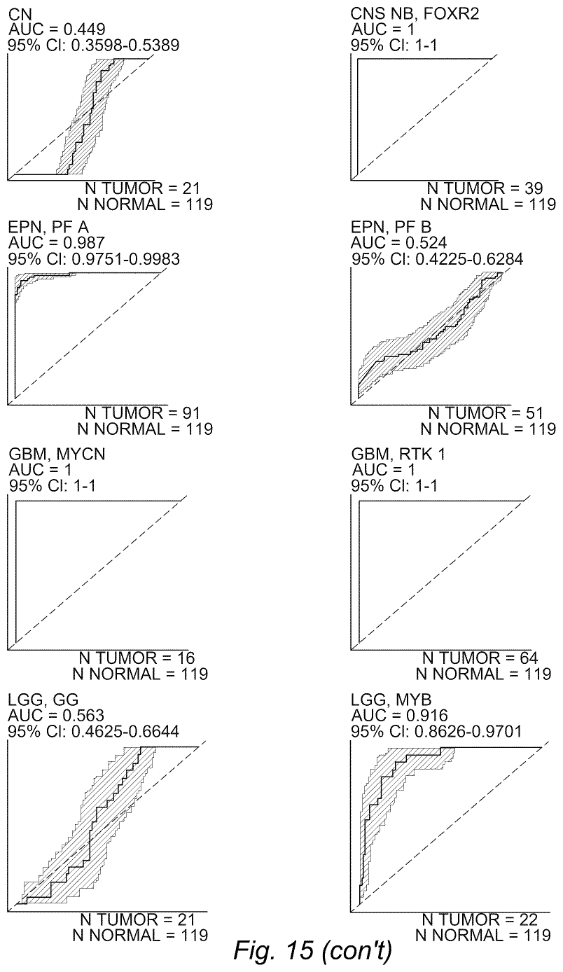

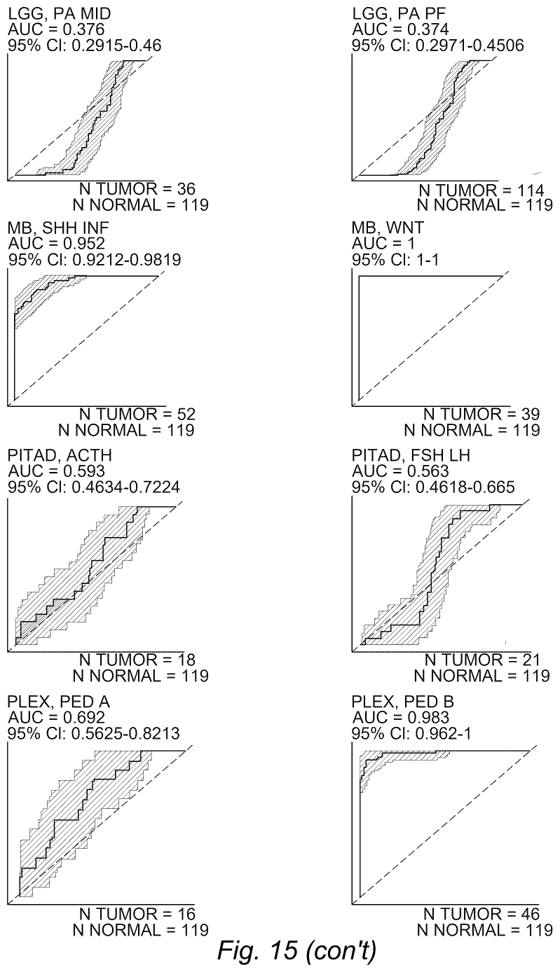

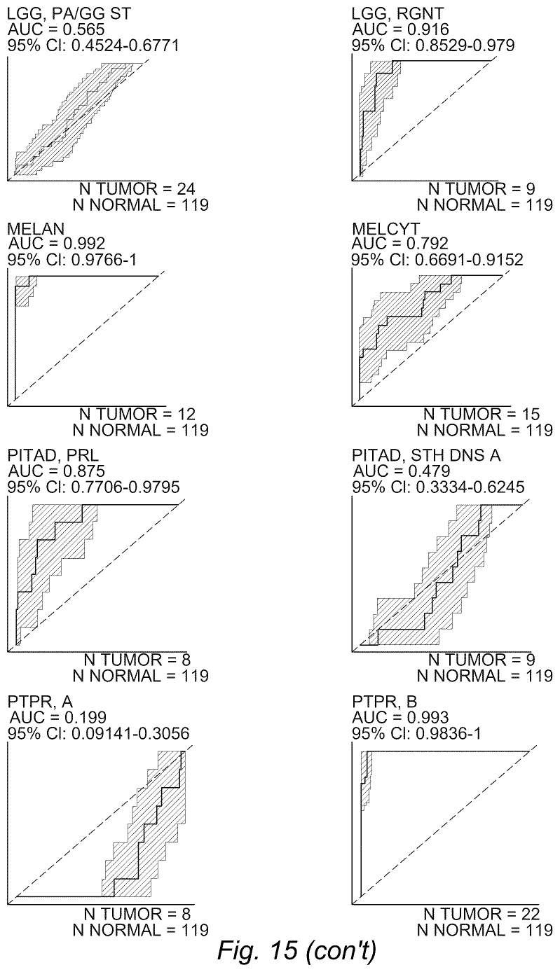

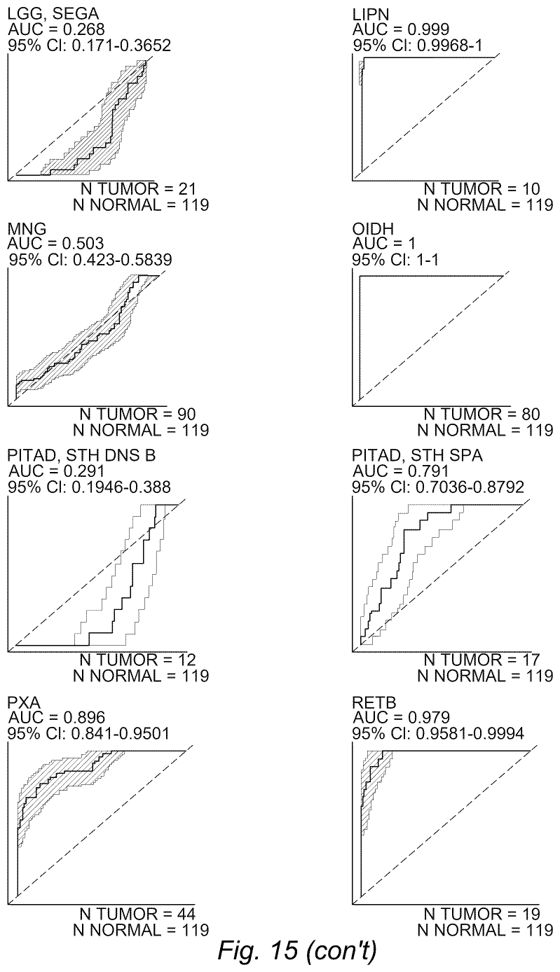

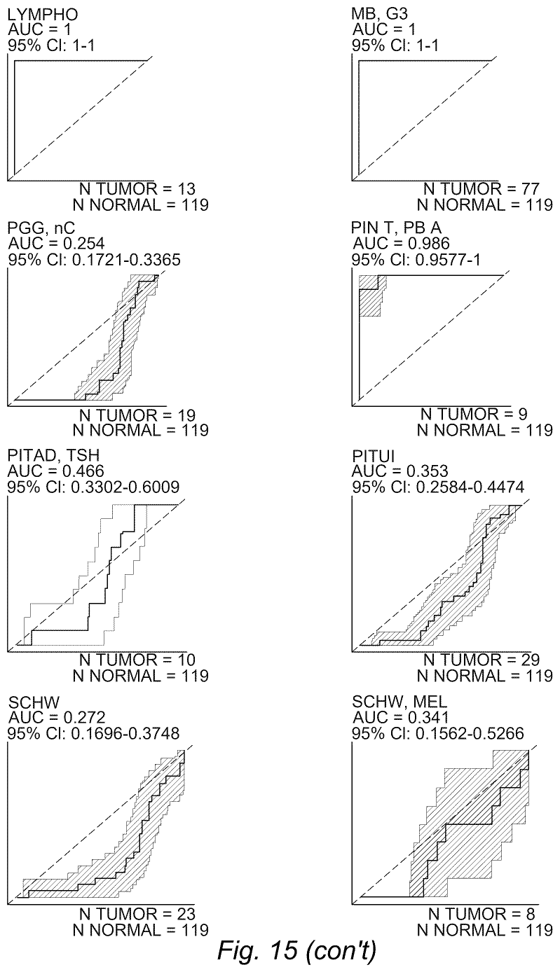

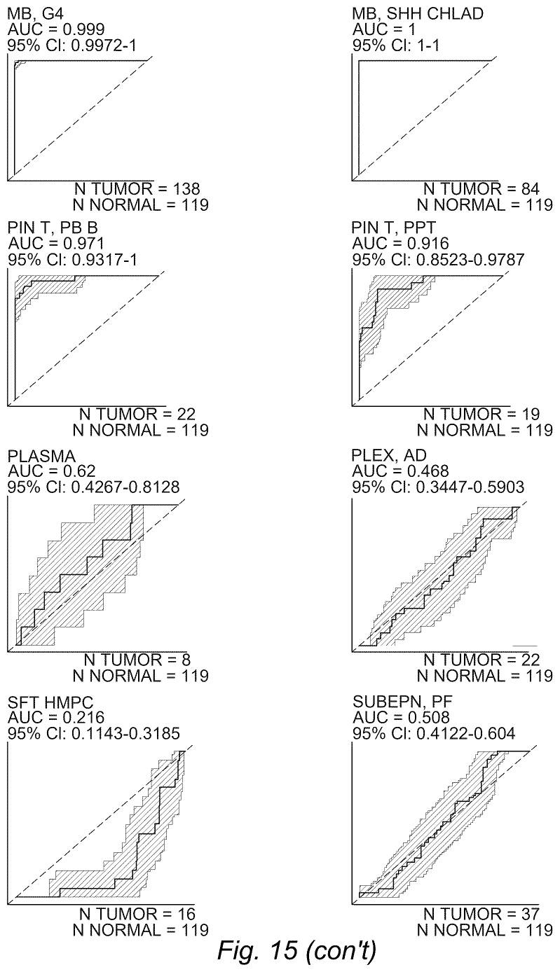

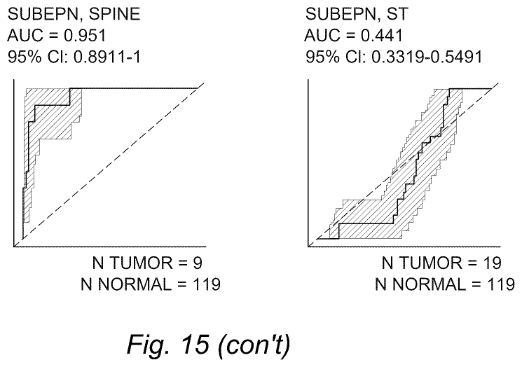

mDMRs discriminate some CNS tumors from normal tissues. Although the mDMR signature was identified in non-CNS tumors, we wanted to determine if these regions were generalizable to pediatric CNS tumors as CNS tumors are the second most common tumor type in children and are high-risk tumors. To do this, we downloaded CNS cancer samples profiled by HM450 arrays generated by Capper et al. Nature 555, 469-474 (2018), hereinafter referred to as ‘DKFZ samples.’ This dataset contains 2682 cancer samples and 119 nonnal control CNS tissues. The dataset comprises 91 unique methylation classes (Capper et al. Table 3)—82 cancers and 9 normal tissue types. We calculated mean beta values at 422 mDMRs that overlap with at least one probe on the HM450 array. We found 44 of 82 tumor types where beta values were significantly different (Wilcoxon p-value<0.05) between tumor and control tissues with concordant directionality in both hypomethylated and hypomethylated mDMRs. Next, beta values fed into the same random forest model as was used to classify TCGA samples and ROC curves were calculated for each methylation class using the 119 control CNS tissues as control for each methylation class. The classifier was able to discriminate between tumor and normal with high sensitivity and specificity (AUC>0.9) in 41/82 CNS tumor types (FIG. 15). Notably, several tumor types in the DKFZ samples are histologically similar to tumor types in the POETIC cohort. For example, three methylation classes—esthesioneuroblastoma (ENB) A, ENB B, and CN NBL—are types of NBL that arise in olfactory nerves and other neural crest cells within the CNS, respectively. All CN NBL cases were classified as tumor (AUC=1) with excellent performance in ENB A (AUC=0.97) and modest performance in ENB B (AUC=0.67). Likewise, all ATRT DKFZ samples, which are histologically similar to POETIC MRT samples, were classified as cancer by the random forest model (AUC=1). Taken together, these results indicate mDMRs are generalizable to some CNS tumor types and are highly generalizable to similar tumor types.

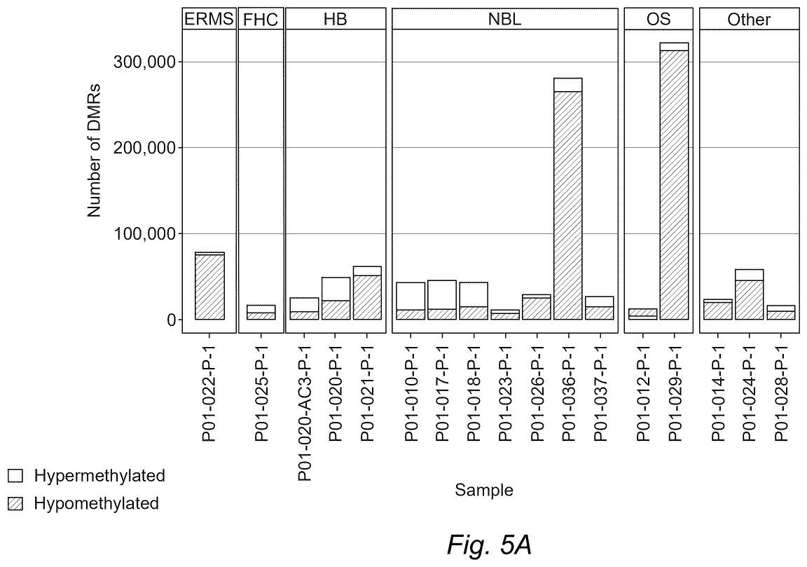

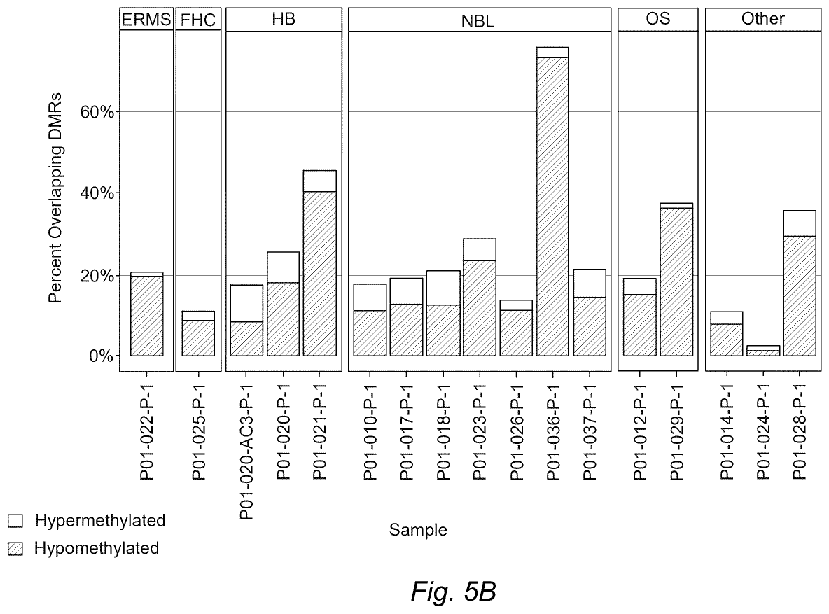

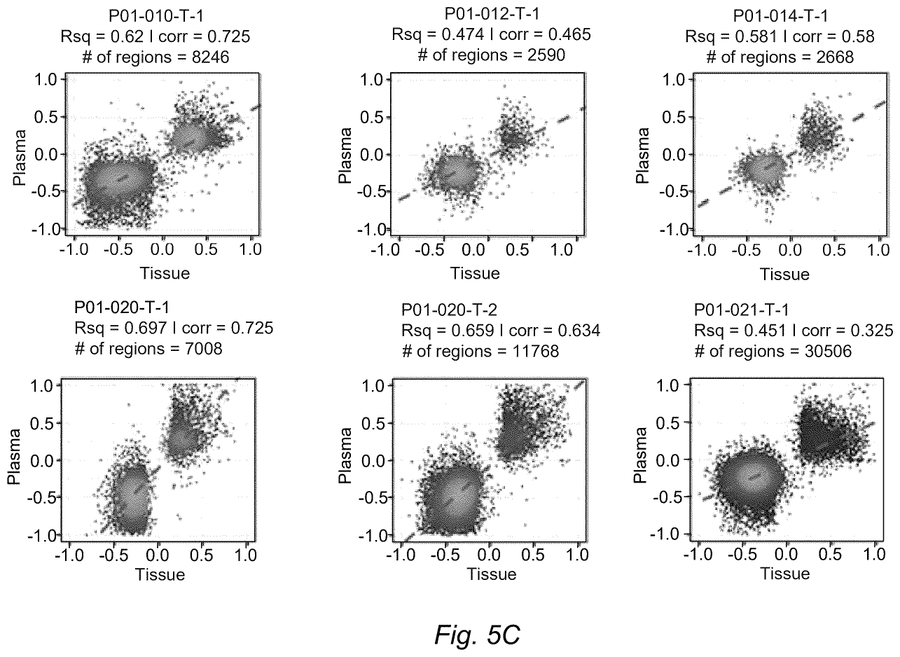

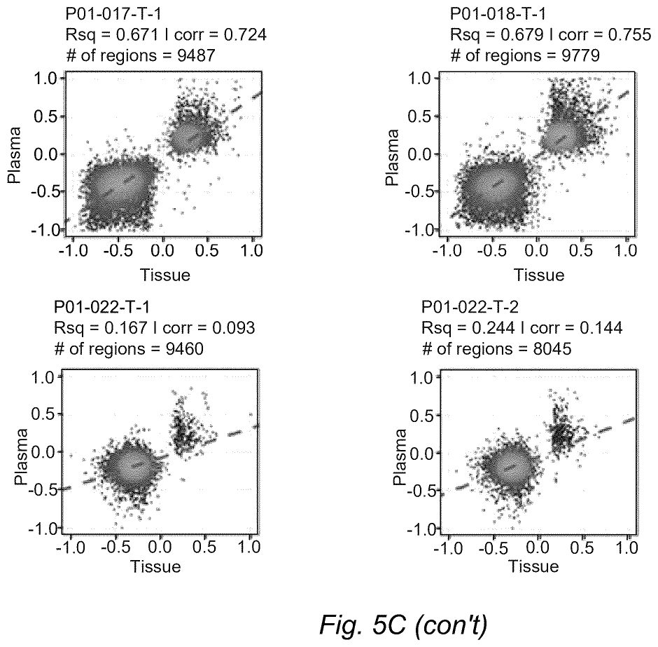

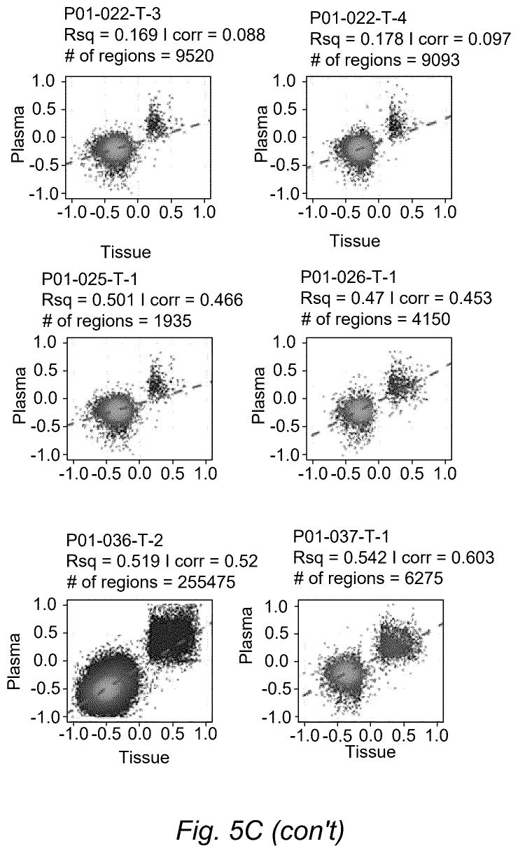

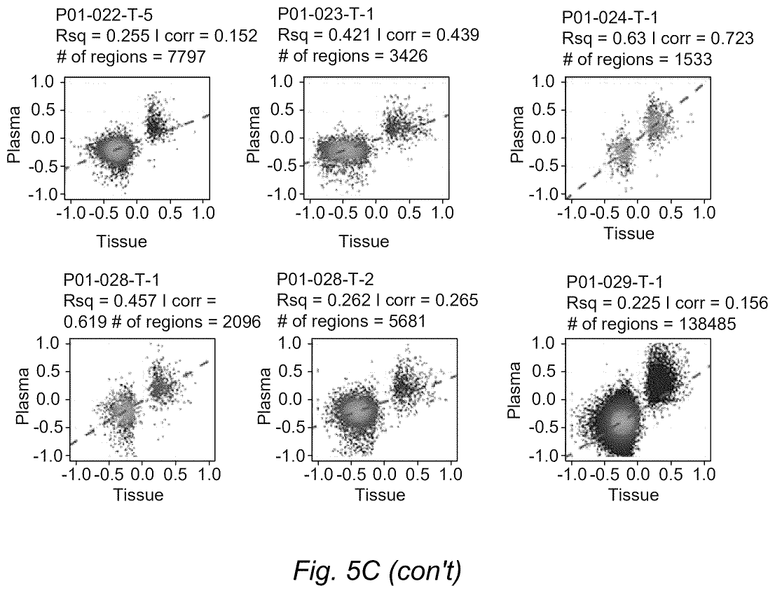

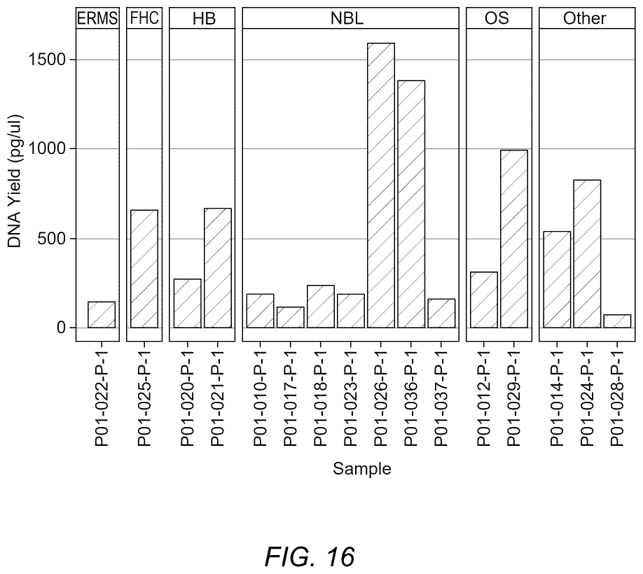

Cell-free DNA methylation reflects tumor tissue methylation patterns. Cell-free DNA methylation is gaining widespread acceptance as an emerging biomarker for liquid biopsies. In the current study, we performed WGBS on 17 individual patient-matched cfDNA samples and 3 normal healthy individuals to determine the extent to which DNA methylation patterns in genomic DNA (gDNA) can be found in cfDNA (Table 2). CfDNA extracted from these samples had an average yield of 20.4 ng/ml (3-2-87.5 ng) (FIG. 16). DMRs were called between cfDNA from cancer patients and the three healthy cfDNA samples and filtered in the same way described above for gDNA from tissue. The median number of DMRs was 42,935 per sample (FIG. 5A) (Table 13).

TABLE 13
DMR summaries from plasma samples. The average, median, minimum, and maximum
number of DMRs by cancer type. The number of cases for each type is also indicated.
The average number of CpGs, median width for the DMRs, and locations with respect
to CpG islands, shores, shelves and open sea are also indicated.

| Dx | nCases | mean.nDMR | median.nDMR | min.nDMR | max.nDMR | mean.nCpG |
|---|---|---|---|---|---|---|
| AE | 1 | 22949 | 22949 | 22949 | 22949 | 12.67750229 |
| ERMS | 1 | 77759 | 77759 | 77759 | 77759 | 13.35114906 |
| FHC | 1 | 16550 | 16550 | 16550 | 16550 | 13.15897281 |
| HB | 3 | 45276 | 49410 | 25063 | 61354 | 13.21040708 |
| NBL | 7 | 68396 | 42935 | 11088 | 280216 | 13.90523955 |
| OS | 2 | 167042 | 167042 | 12464 | 321619 | 15.14526257 |
| Teratoma | 1 | 16674 | 16674 | 16674 | 16674 | 12.45717884 |
| WT | 1 | 58224 | 58224 | 58224 | 58224 | 14.07414468 |

| Dx | median.width | CpG.Island.pct | CpG.Shore.pct | CpG.Shelf.pct | Open.Sea.pct |
|---|---|---|---|---|---|
| AE | 561 | 6.985053815 | 7.860908972 | 4.819382108 | 80.3346551 |
| ERMS | 740 | 3.038876529 | 6.170346841 | 4.350621793 | 86.44015484 |
| FHC | 432 | 12.03625378 | 13.95770393 | 5.788519637 | 68.21752266 |
| HB | 544 | 6.709577272 | 10.6873355 | 5.545714491 | 77.05737274 |
| NBL | 540 | 7.589508968 | 10.60531259 | 5.43385086 | 76.37132758 |
| OS | 679 | 7.968881434 | 8.841953628 | 4.926489616 | 78.26267532 |
| Teratoma | 411 | 11.83279357 | 10.89720523 | 5.229698932 | 72.04030227 |
| WT | 674 | 5.812036274 | 10.29128882 | 5.17484199 | 78.72183292 |

We determined the number of overlapping DMRs between cfDNA and gDNA in the patient-matched samples. To count as an overlapping DMR, at least 3 CpGs methylated in the same direction needed to be present in both sample types. The mean percentage of overlapping DMRs between the cfDNA and gDNA (FIG. 5B) was 24.9% (Bettegowda et al., Sci Transl Med 6, 224ra224 (2014)). Some notable exceptions include the following samples which had exceptional overlap between cfDNA DMRs and gDNA DMRs: P01-036 (NBL, 76.0%), P0-021 (hepatoblastoma (HB), 45.6%), P01-028 (Teratoma, 35.8%) and P01-029 (OS, 37.4%). The DMRs that overlapped between gDNA and cfDNA were positively correlated (FIG. 5C) with an average R² of 0.45.

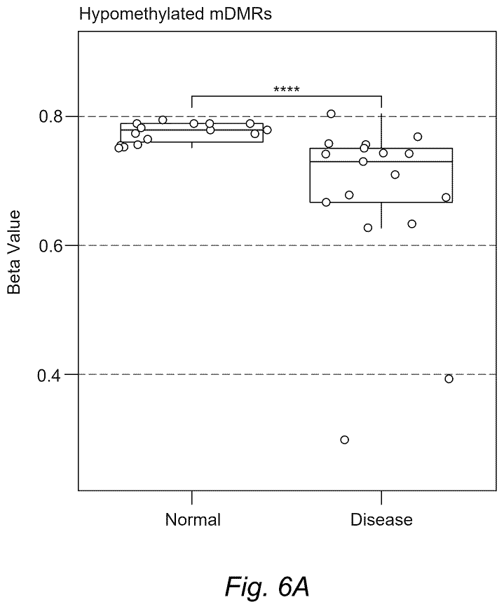

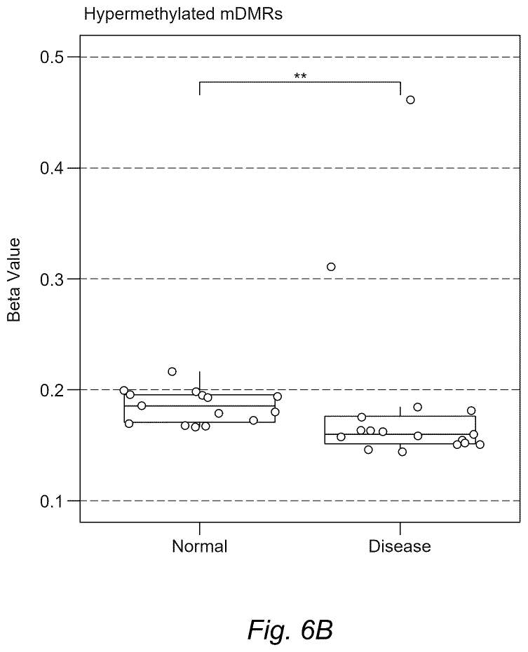

Pan-pediatric mDMRs detectable in cfDNA. To determine whether the 905 mDMRs (402 hypomethylated and 503 hypermethylated) across tumor types (FIG. 2) could differentiate between tumor and normal in cfDNA, we performed WGBS in 17 pediatric cancer cases and 15 healthy controls and calculated mean beta values across all hyper/hypomethylated mDMRs identified in patient-matched tumor tissue, in each WGBS cfDNA sample. Mean methylation for hypomethylated mDMRs identified in tissue was significantly lower in cfDNA cancer samples compared to normal with an average methylation difference of 0.10 (p<0.0001) (FIG. 6A). However, mean methylation for hypermethylated regions identified in tissue was also unexpectedly lower in cfDNA cancer samples compared to normal (FIG. 6B). For this reason, we focused further validation of mDMRs on hypomethylated regions only, and used a targeted hybridization probe capture assay designed to the 402 hypomethylated mDMRs.

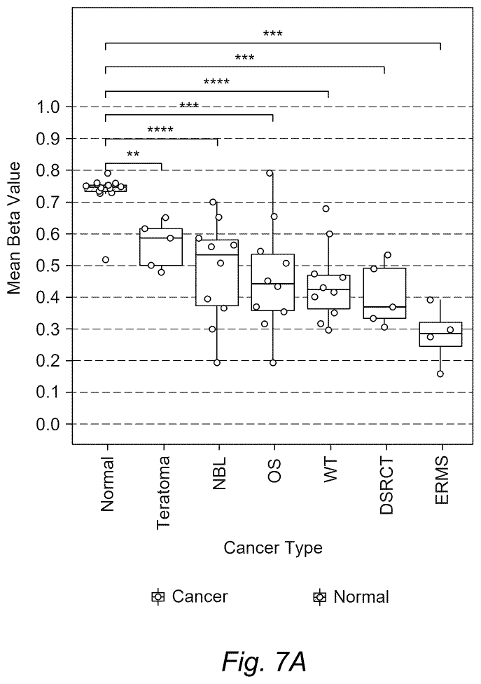

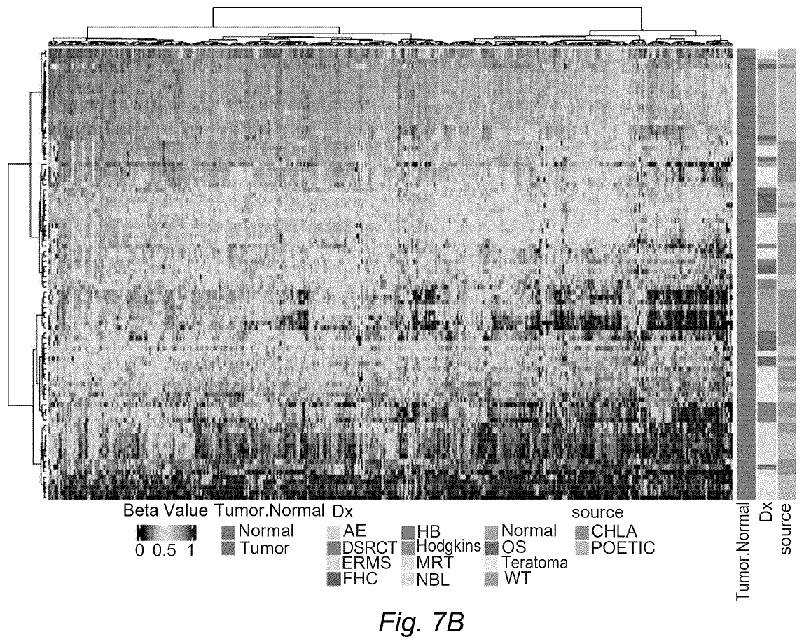

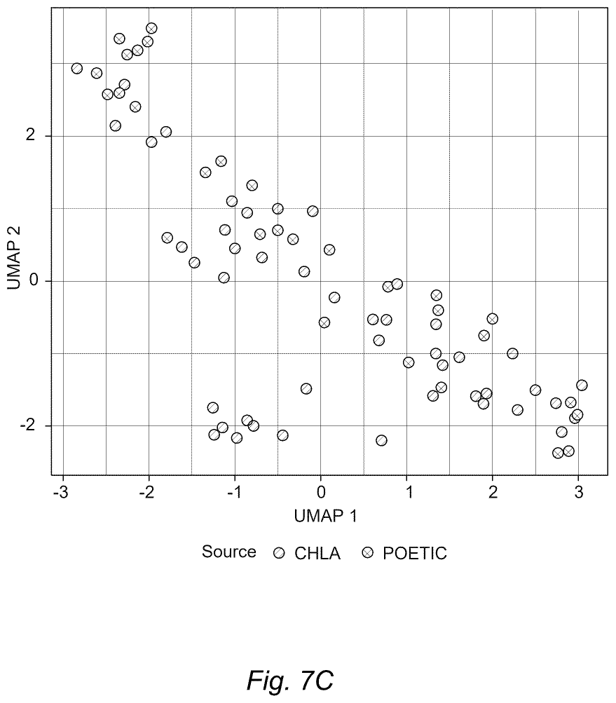

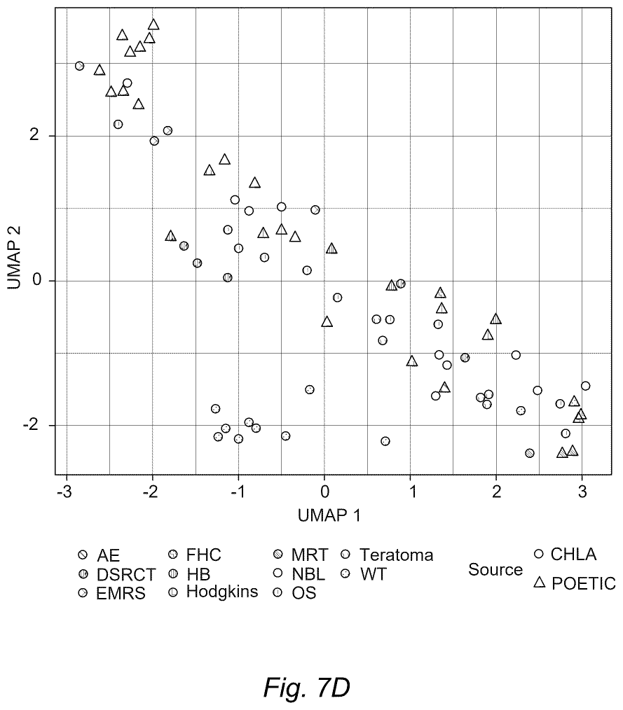

We acquired an additional cohort of 44 pediatric cancer samples from Children's Hospital Los Angeles (CHLA), although cfDNA was not available for these. This cohort was comprised of 6 cancer types (NBL, OS, WT, desmoplastic small round cell tumor (DSRCT), embryonal rhabdomyosarcoma (ERMS), and teratoma) (Table 14). We found that each of these tumor types was also significantly hypomethylated with respect to normal tissue (FIG. 7A). Hierarchical clustering showed CHLA samples clustering with POETIC samples and separated from normal tissue samples (FIG. 7B). Furthermore, UMAP analysis did not show a source effect (FIG. 7C) and showed like-tumor types clustering together, regardless of source (FIG. 7D)).

TABLE 14
CHLA sample summary. All cases from CHLA with demographic
information and cancer type.

| BSI.ID | Tumor Type | Age at surgery | Sex |
|---|---|---|---|
| AAA000153 | DSRCT | 16 | M |
| AAA010161 | DSRCT | 7 | M |
| AAA011812 | DSRCT | 10 | F |
| AAA011841 | DSRCT | 10 | F |
| AAA012743 | DSRCT | 10 | M |
| AAA009588 | Neuroblastoma | 0 | M |
| AAA009596 | Neuroblastoma | 2 | F |
| AAA009714 | Neuroblastoma | 10 | M |
| AAA009727 | Neuroblastoma | 3 | F |
| AAA009772 | Neuroblastoma | 6 | F |
| AAA009923 | Neuroblastoma | 2 | F |
| AAA009935 | Neuroblastoma | 2 | F |
| AAA009955 | Neuroblastoma | 2 | M |
| AAA009988 | Neuroblastoma | 3 | M |
| AAA010103 | Neuroblastoma | 1 | M |
| AAA002841 | Osteosarcoma | 14 | M |
| AAA003165 | Osteosarcoma | 14 | M |
| AAA009448 | Osteosarcoma | 16 | M |
| AAA009738 | Osteosarcoma | 19 | M |
| AAA009752 | Osteosarcoma | 16 | M |
| AAA009886 | Osteosarcoma | 13 | M |
| AAA010356 | Osteosarcoma | 11 | M |
| AAA012396 | Osteosarcoma | 13 | F |
| AAA013412 | Osteosarcoma | 8 | F |
| AAA016160 | Osteosarcoma | 5 | F |
| AAA004012 | Rhabdomyosarcoma | 13 | M |
| AAA010286 | Rhabdomyosarcoma | 9 | F |
| AAA010510 | Rhabdomyosarcoma | 5 | M |
| AAA012345 | Rhabdomyosarcoma | 8 | M |
| AAA002850 | Teratoma | 0 | F |
| AAA002915 | Teratoma | 8 | M |
| AAA004055 | Teratoma | 0 | F |
| AAA005397 | Teratoma | 9 | F |
| AAA015908 | Teratoma | 2 | M |
| AAA002749 | Wilms | 3 | F |
| AAA002750 | Wilms | 6 | F |
| AAA002786 | Wilms | 1 | F |
| AAA002795 | Wilms | 7 | M |
| AAA002813 | Wilms | 3 | M |
| AAA002822 | Wilms | 4 | F |
| AAA002823 | Wilms | 5 | M |
| AAA009770 | Wilms | 3 | M |
| AAA009890 | Wilms | 3 | M |
| AAA010032 | Wilms | 2 | F |

Identification of DNA methylation deserts in neuroblastoma. After identifying and validating common focal epigenetic alterations, we turned our focus to large scale genomic and epigenomic alterations such as partially methylated domains (PMDs; large regions of hypomethylation spanning several hundred kilobases to several megabases, which have been described as a common epigenetic alteration in cancer) and copy number variants. PMD-positive samples had an average of 98 PMDs larger than 1 megabase. We also identified multi-kilobase regions that were significantly more hypomethylated, beyond the levels traditionally reported for PMDs, which we refer to as DNA methylation deserts. These deserts were found in 5 of 9 neuroblastomas, but were not identified in any other tumor type. Methylation deserts were characterized by DNA methylation beta value averages less than 0.2, which is significantly lower than that seen in traditional PMDs which typically have a beta value of 0.7 (Lister et al., Nature 462, 315-322 (2009)). PMDs and methylation deserts were observed in cfDNA in P01-010, P01-029, and P01-036.

Interestingly, forkhead box genes FOXA1 and FOXP2 were found within the methylation deserts of the 5 NBL samples. Traditional PMDs were also detected in several other tumors including 3 additional NBL and 11 other samples, including the 5 EMRS samples from one patient, 3 OS, and 2 HB. Permutation testing (Gel et al., Bioinformatics 32, 289-291 (2016)) was used to evaluate whether these PMDs and methylation deserts were associated with copy number variants; we did not find a significant association. We identified 178 regions that contained PMD calls in more than 50% of samples; these regions ranged from 5 kb to 2.1 Mb with an average width of 308 kb and an average of 2699 CpGs per region. Hierarchical clustering of samples based on overall methylation values within these 178 consensus regions separated the 5 NBL samples with deserts from the 14 samples with PMDs and from the remaining samples (with no significant PMDs called).

Copy Number Estimation by WGBS in gDNA from tumor tissue. WGBS is primarily used to measure DNA methylation across the entire genome but can also be used reliably in determining copy number variants (CNVs). In this study, we conducted copy number analysis from WGBS data, which revealed CNVs in recurrent tumors consistent with known copy number variants in various primary tumor types studied. Specifically, we identified 1q gain—a structural variant associated with poor prognosis—in 18 of the 31 solid tumor samples (1/1 anaplastic ependymoma (AE), 1/1 DSRCT, 7/10 NBL, 5/5 ERMS, and 3/3 HB). The strong 8q gain in the two hepatoblastoma tumor samples is likewise associated with poor prognosis. 8q was amplified in P01-020 with an average copy number of 3.7 in the first sample collected (P01-020-T-1) and 4.8 in the second sample collected six months later (P01-020-T-2). The copy number profile in these two samples was largely consistent; however, 13q loss and 18q gain were observed at the second time point but not the first. In NBL samples, we observed 1p loss in 4/10 samples, 17q gain in all samples, 11q loss in 6/10 samples, and 3p loss in 5/10 samples. All of these structural variants are relatively common in NBL. In OS samples (P01-012, P01-016, P01-029, and P01-030), copy number changes were widespread and chaotic, a characteristic of OS which often has numerous structural alterations and chromothripsis. The four samples with a hypermethylator phenotype samples had no large chromosomal aberrations: P01-019-T-1 (MRT), P01-019-T-2 (MRT), P01-027-T1 (Hodgkin's lymphoma), and P01-024-T-1 (WT).

We also detected numerous focal deletions and amplifications that are known to drive the specific tumor types in our study. We were able to observe N-MYC amplification in two of ten NBL samples. N-MYC amplifications in NBL occur in 20-25% of NBLs and are associated with poor prognosis. We identified homozygous loss of SMARCB1 in the 2 MRT samples, which is a known driver mutation for that tumor type. We observed homozygous loss of MMP11 in MRT. MMP11 CNVs have been reported in NBL and WT in the catalogue of somatic mutations in cancer (COSMIC); however, MMP11 CNVs in MRT have not been reported. All samples from P01-022 (ERMS) had PTEN loss, a CNV that has been previously reported and is associated with an aggressive cancer phenotype. This homozygous loss in P01-022 also contains ATAD1 which has been associated with cancer cell progression. PTEN loss in this ERMS tumor likely represents a driver tumor event present in all cells.

Detection of CNVs in cfDNA is less sensitive than cfDNA methylation. Next, we used WGBS data to analyze CNVs in cfDNA. Unlike many known CNVs found in tissue gDNA, CNVs were largely not observable in cfDNA. Five samples from three patients represented notable exceptions, where CNV calls were broadly consistent with CNV detection from tissue gDNA. In one such example (P01-020), the 1q and 8q gains observed in 2 tissue samples were observed in cfDNA. Blood from this patient was taken at the time the first tumor sample (P01-020-T1) was collected and both samples had a similar CNV profile. The second tumor sample (P01-020-T2) was collected at a later date and showed a 13q loss not seen in the first sample (T1) or its cfDNA. In P01-036 (NBL) the cfDNA CNV calls also closely matched tumor sample CNV data, with gains on 1q, 2p, 7, 9q, 12q, 13q, 17q and losses on 1p, 3p, 4q/p, 11q, 17q, and 19q. Interestingly the cfDNA was able to resolve some CNVs not seen in the tissue, namely losses in 10p and 15q. This was also true for P01-029 where there were CNVs in plasma not seen in gDNA. In addition, we were able to identify focal N-MYC amplification in P01-026 but not in P01-023 as previously identified in gDNA. The reasons why CNV calls in cfDNA were concordant with gDNA in only a few samples are unknown, but were unrelated to tumor cfDNA yields, a proxy for tumor burden (FIG. 16). Overall, this analysis revealed that cfDNA methylation is more sensitive to detect tissue-associated alterations than CNV analysis in plasma.

In this study we set out to determine the set of DMRs common to multiple non-CNS pediatric solid tumors. To do this we present WGBS data on 31 tumor and 13 normal tissue samples from recurrent pediatric non-CNS solid cancers representing 11 different tumor types. We further validated DNA methylation findings in 518 pediatric cancer samples from TARGET and 6,426 adult cancer samples from TCGA, along with an additional 44 pediatric cancer tissue samples examined through a targeted hybridization probe capture assay. Additionally, we examined corresponding cfDNA methylation from 17 patient-matched plasma samples. Previous studies have identified DNA methylation changes in many of the pediatric cancers evaluated in this study, including NBL, OS, WT, AE, HB, ERMS, and fibrolamellar hepatocellular carcinoma, however methylation analysis on recurrent pediatric cancer cases has been limited. Novel to our study is the finding that in addition to tumor-specific changes, DNA methylation patterns were also shared across the myriad of tumor types analyzed regardless of whether a tumor was from a primary or recurrent tumor. And it has been demonstrated that there are DNA methylation changes common to multiple adult cancer types, which are frequently associated with tumor suppressor genes, and often associated with survival. However, similar findings in pediatric cancers have been scarce. Given the rarity of many pediatric cancers, identifying DNA methylation patterns across multiple pediatric cancer types might have great value for developing biomarker approaches for early detection and/or disease recurrence through MRD detection.

To find DNA methylation commonalities between cancer types, we identified a minimal region of differential methylation common to multiple samples across cancer types which we termed minimally differentially methylated regions—mDMRs. These mDMRs were significantly differentially methylated in multiple cancer types, including very rare tumors like DSRCT, when compared to nonnal tissue. We also evaluated 518 pediatric cancer samples from the TARGET database, where we detected methylation alterations in the mDMR regions consistent with those observed in POETIC samples, indicating this signature is generalizable. Furthermore, since this signature is derived from recurrent patients, it may reflect a method to conduct recurrence monitoring via MRD detection in pediatric cancer. Finally, this signature also demonstrated high sensitivity and specificity when tested in adult cancer samples from TCGA, suggesting that these mDMRs could also serve as a pan-cancer detection marker in adult cancers. Another noteworthy finding of this study was that methylation profiles of recurrent POETIC samples largely resemble those of primary tumors sourced from TARGET. Previous work has shown that primary and recurrent tumors have similar methylation profiles, however, this has not been previously reported in pediatric cancer, albeit the TARGET data also indicates this to be true.

Among the tumor-specific DNA methylation changes of interest was the identification of DNA methylation deserts in a subset of NBL cases, where large regions of DNA methylation are essentially eroded. The functional significance of these DNA methylation deserts is unknown but are distinct from previously reported PMDs in the degree of the observed loss of DNA methylation. Previously, it was found that PMDs are associated with CpG island methylation in breast cancer; PMDs have also been associated with lanina-associated domains (LADs). Increased expression of FOXA1, one of the few genes found in DNA methylation deserts, has been previously linked to late recurrence. Further research is needed to explore possible mechanisms of these desert regions in NBL. Another tumor-specific methylation change we observed was strong global hypermethylation in MRT—an atypical cancer phenotype in pediatric and adult tumors. Hypermethylation in cancer typically occurs in the promoter regions of tumor suppressor genes and in CpG islands, where it is associated with poor prognosis in many adult and pediatric cancers and can be associated with CIMP. However, global hypermethylation is rarely reported in cancer. While MRT has been previously shown to have focal hypermethylation, the global hypermethylator phenotype we observed has not been previously described. And it has been found that SMARCB1 restoration in MRT cell lines resulted in widespread chromatin activation. Conversely this hypermethylator phenotype could be linked to genome-wide chromatin inactivation but future studies would be needed to evaluate if this is true.

Unlike adult cancers, most cases of pediatric cancer can be traced to a single genetic driver, and these genetic alterations tend to be highly tumor-specific. For example, the loss of SMARCB1 specifically leads to the development of MRT. Specific gene fusions cause alveolar rhabdomyosarcoma (PAX3/7-FOXO1), Ewing's sarcoma (EWS-FLI1), and CML (BCR-ABL1). Mutations to certain genes such as RB are specific to retinoblastoma and OS, while mutations in TP53 are extremely common in multiple pediatric cancers. WGBS is the gold standard assay for methylation evaluation as it evaluates every CpG in the genome, but we also used it for copy number estimation—maximizing data generation from each sample and enabling multi-omic analysis from the same sample and aliquot, which also minimizes sampling bias and reduces cost. This is particularly useful when dealing with rare tumor types or sample types with very little available DNA (such as cfDNA). CNV analysis from WGBS identified both large-scale alterations and focal gains/losses such as MYCN gain in NBL, SMARCB1 loss in MRT, and PTEN loss in ERMS.

A key component of this study was evaluating the degree to which genomic alterations in solid tumors were reflected in cfDNA, as cfDNA analysis is rapidly becoming a major tool for non-invasive screening, diagnosis, treatment, and monitoring of human tumors. In renal cell carcinoma it was found that cfDNA methylation (using MeDIP-seq) was far more sensitive and specific than cfDNA SNV markers. In the current study, we found that detecting tumor tissue-derived DNA methylation using WGBS was superior to detecting CNVs in cfDNA. In this study, only 3/17 cfDNA samples reflected the copy number profile of the tumor, whereas the remaining samples lacked much of the signal found in tissue. By contrast, we found cfDNA methylation to resemble tissue DNA methylation more robustly, where on average approximately 25% of DMRs were common to tissue and plasma in each sample. We also found that hypermethylated regions were less likely to be retained in plasma and were often found to be hypomethylated. While the reasons for this are unclear, the implications are significant as it would biomarker development strategies.

In summary, this disclosure provides a comprehensive analysis of multiple pediatric cancers using WGBS. We identified a pan-cancer methylation signature, detectable in cfDNA, common to multiple pediatric cancer types, including extremely rare neoplasms such as DSRCT and MRT. We also used WGBS to detect CNVs, in order to directly compare CNV and methylation detection from the same sample and aliquot. We found that DNA methylation was superior to CNV at detecting tumor-specific signal in cfDNA. The pan-cancer cfDNA methylation signature in this study has potential utility in minimal residual disease monitoring and early detection and warrants further investigation in both pediatric and adult cancer.

### Example 2 Materials and Methods

Sample collection. Samples were obtained under written informed parental consent from the Pediatric Oncology Experimental Therapeutics Investigators' Consortium (POETIC) at Memorial Sloan Kettering Cancer Center (New York, USA) from patients with a wide range of recurrent pediatric cancers (Table 2). Tissue samples were flash-frozen after resection. Peripheral blood samples were drawn pre-operatively in EDTA purple-top tubes and the plasma was harvested.

TABLE 2A
Patient summary. All unique samples from the POETIC cohort used for
WGBS. The diagnosis for each patient is indicated, along with
the sample type and resection location where applicable.

| Sample.ID | Patient.ID | Diagnosis | Sample Type | Resection Location |
|---|---|---|---|---|
| P01-010-P-1 | P01-010 | Neuroblastoma | PLASMA | Peripheral Blood |
| P01-010-N-1 | P01-010 | Neuroblastoma | NORMAL_TISSUE | Adjacent tissuc normal |
| P01-010-T-1 | P01-010 | Neuroblastoma | CANCER_TISSUE | Unspecified |
| P01-012-P-1 | P01-012 | Osteosarcoma | PLASMA | Peripheral Blood |
| P01-012-N-1 | P01-012 | Osteosarcoma | NORMAL_TISSUE | Adjacent tissue normal |
| P01-012-T-1 | P01-012 | Osteosarcoma | CANCER_TISSUE | Lung |
| P01-014-P-1 | P01-014 | Anaplastic | PLASMA | Peripheral Blood |
| | | Ependymoma | | |
| P01-014-T-1 | P01-014 | Anaplastic | CANCER_TISSUE | Unspecified |
| | | Ependymoma | | |
| P01-015-N-1 | P01-015 | Fibrolamellar | NORMAL_TISSUE | Adjacent tissue normal |
| | | Hepatocellular | | |
| | | Carcinoma | | |
| P01-015-T-1 | P01-015 | Fibrolamellar | CANCER_TISSUE | Unspecified |
| | | Hepatocellular | | |
| | | Carcinoma | | |
| P01-016-N-1 | P01-016 | Osteosarcoma | NORMAL_TISSUE | Adjacent tissue normal |
| P01-016-T-1 | P01-016 | Osteosarcoma | CANCER_TISSUE | Lung |
| P01-017-P-1 | P01-017 | Neuroblastoma | PLASMA | Peripheral Blood |
| P01-017-T-1 | P01-017 | Neuroblastoma | CANCER_TISSUE | Unspecified |
| P01-018-P-1 | P01-018 | Neuroblastoma | PLASMA | Peripheral Blood |
| P01-018-T-1 | P01-018 | Neuroblastoma | CANCER_TISSUE | Abdomen |
| P01-019-N-1 | P01-019 | Malignant | NORMAL_TISSUE | Adjacent tissue normal |
| | | Rhabdoid Tumor | | |
| P01-019-T-1 | P01-019 | Malignant | CANCER_TISSUE | Abdomen |
| | | Rhabdoid Tumor | | |
| P01-019-T-2 | P01-019 | Malignant | CANCER_TISSUE | Abdomen |
| | | Rhabdoid Tumor | | |
| P01-020- | P01-020 | Hepatoblastoma | PLASMA | Peripheral Blood |
| AC3- | | | | |
| P-1 | | | | |
| P01-020-P-1 | P01-020 | Hepatoblastoma | PLASMA | Peripheral Blood |
| P01-020-N-1 | P01-020 | Hepatoblastoma | NORMAL_TISSUE | Adjacent tissue normal |
| P01-020-T-1 | P01-020 | Hepatoblastoma | CANCER_TISSUE | Unspecified |
| P01-020-T-2 | P01-020 | Hepatoblastoma | CANCER_TISSUE | Lung |
| P01-021-P-1 | P01-021 | Hepatoblastoma | PLASMA | Peripheral Blood |
| P01-021-N-1 | P01-021 | Hepatoblastoma | NORMAL_TISSUE | Adjacent tissue normal |
| P01-021-T-1 | P01-021 | Hepatoblastoma | CANCER_TISSUE | Lung |
| P01-022-P-1 | P01-022 | Embryonal | PLASMA | Peripheral Blood |
| | | rhabdomyosarcoma | | |
| P01-022-N-1 | P01-022 | Embryonal | NORMAL_TISSUE | Adjacent tissuc normal |
| | | rhabdomyosarcoma | | |
| P01-022-T-1 | P01-022 | Embryonal | CANCER_TISSUE | Omentum |
| | | rhabdomyosarcoma | | |
| P01-022-T-2 | P01-022 | Embryonal | CANCER_TISSUE | Diaphragm |
| | | rhabdomyosarcoma | | |
| P01-022-T-3 | P01-022 | Embryonal | CANCER_TISSUE | Sinus venous mass |
| | | rhabdomyosarcoma | | |
| P01-022-T-4 | P01-022 | Embryonal | CANCER_TISSUE | Pelvic mass |
| | | rhabdomyosarcoma | | |
| P01-022-T-5 | P01-022 | Embryonal | CANCER_TISSUE | Sigmoid colon |
| | | rhabdomyosarcoma | | |
| P01-023-P-1 | P01-023 | Neuroblastoma | PLASMA | Peripheral Blood |
| P01-023-T-1 | P01-023 | Neuroblastoma | CANCER_TISSUE | Mediastinal mass |
| P01-024-P-1 | P01-024 | Wilm's Tumor | PLASMA | Peripheral Blood |
| P01-024-N-1 | P01-024 | Wilm's Tumor | NORMAL_TISSUE | Adjacent tissue normal |
| P01-024-T-1 | P01-024 | Wilm's Tumor | CANCER_TISSUE | Unspecified |
| P01-025-P-1 | P01-025 | Fibrolamellar | PLASMA | Peripheral Blood |
| | | Hepatocellular | | |
| | | Carcinoma | | |
| P01-025-T-1 | P01-025 | Fibrolamellar | CANCER_TISSUE | Unspecified |
| | | Hepatocellular | | |
| | | Carcinoma | | |
| P01-026-P-1 | P01-026 | Neuroblastoma | PLASMA | Peripheral Blood |
| P01-026-T-1 | P01-026 | Neuroblastoma | CANCER_TISSUE | Adrenal Gland |
| P01-027-T-1 | P01-027 | Hodgkin's | CANCER_TISSUE | Unspecified |
| | | Lymphoma | | |
| P01-028-P-1 | P01-028 | Immature teratoma | PLASMA | Peripheral Blood |
| P01-028-N-1 | P01-028 | Immature teratoma | NORMAL_TISSUE | Adjacent tissue normal |
| P01-028-T-1 | P01-028 | Immature teratoma | CANCER_TISSUE | Cul De Sac |
| P01-028-T-2 | P01-028 | Immature teratoma | CANCER_TISSUE | Ileum |
| P01-029-P-1 | P01-029 | Osteosarcoma | PLASMA | Peripheral Blood |
| P01-029-N-1 | P01-029 | Osteosarcoma | NORMAL_TISSUE | Adjacent tissue normal |
| P01-029-T-1 | P01-029 | Osteosarcoma | CANCER_TISSUE | Retroperitoneum |
| P01-030-N-1 | P01-030 | Osteosarcoma | NORMAL_TISSUE | Adjacent tissue normal |
| P01-030-T-1 | P01-030 | Osteosarcoma | CANCER_TISSUE | Lung |
| P01-033-T-1 | P01-033 | Neuroblastoma | CANCER_TISSUE | Retroperitoneum |
| P01-035-T-1 | P01-035 | Desmoplastic | CANCER_TISSUE | Gastric lesion |
| | | Small Round | | |
| | | Cell Tumor | | |
| P01-036-P-1 | P01-036 | Neuroblastoma | PLASMA | Peripheral Blood |
| P01-036-T-2 | P01-036 | Neuroblastoma | CANCER_TISSUE | Abdomen |
| P01-037-P-1 | P01-037 | Neuroblastoma | PLASMA | Peripheral Blood |
| P01-037-N-1 | P01-037 | Neuroblastoma | NORMAL_TISSUE | Adjacent tissue normal |
| P01-037-T-1 | P01-037 | Neuroblastoma | CANCER_TISSUE | Unspecified |
| P01-040-T-1 | P01-040 | Neuroblastoma | CANCER_TISSUE | Unspecified |
| P01-0813-N | P01-0813 | Healthy donor | PLASMA | Peripheral Blood |
| P01-2013-N | P01-2013 | Healthy donor | PLASMA | Peripheral Blood |
| P01-2714-N | P01-2714 | Healthy donor | PLASMA | Peripheral Blood |

All participants in the study were patients with recurrent pediatric cancer. In total, 44 tissue samples were collected including 31 tumor samples and 13 matching adjacent normal samples from 24 patients. All tissue samples were collected during surgical resection of the recurrent tumor. 17 patient-matched plasma samples were collected, with 3 additional plasma samples from young healthy individuals purchased from Conversant Bio (Table 2). Twelve additional cfDNA healthy controls from adults were included for model building in cfDNA, detailed below (Conversant Bio). Patients ranged in age from 18 months to 35 years. In total, 11 different cancer types were included in the study: neuroblastoma (NBL, n=9), osteosarcoma (OS, n=4; 3 from pulmonary metastasis, 1 from retroperitoneal metastasis), fibrolamellar hepatocellular carcinoma (FHC, n=2), hepatoblastoma (HB, n=2; both resected from the liver, with one additional sample from a pulmonary metastasis), anaplastic ependymoma (n=1), desmoplastic small round cell tumor (n=1: from a gastric lesion), malignant rhabdoid tumor (MRT, n=1; 2 samples from an abdominal lesion), Hodgkin's lymphoma (n=1; from a lung biopsy), embryonal rhabdomyosarcoma (ERMS, n=1; with 5 samples from omentum, diaphragm, sinus venous, pelvic, and sigmoid colon lesions), immature teratoma (n=1: with 2 samples from cul de sac and ileum lesions), and Wilms' tumor (n=1).

TABLE 2B
Patient summary. All unique cases from the POETIC cohort with demographic information, diagnosis, and sample
source. Number of tumor tissue, normal tissue and plasma samples analyzed per patient is indicated.

| Patient | | | Tumor | Normal | Plasma | | Diagnosis | |
|---|---|---|---|---|---|---|---|---|
| ID | Sex | Age | Samples | Samples | Samples | Diagnosis | Abbreviation | Source |
| P01-010 | F | 35 | 1 | 1 | 1 | Neuroblastoma | NBL | POETIC |
| P01-012 | M | 16 | 1 | 1 | 1 | Osteosarcoma | OS | POETIC |
| P01-014 | F | 10 | 1 | 0 | 1 | Anaplastic Ependymoma | AE | POETIC |
| P01-015 | M | 15 | 1 | 1 | 0 | Fibrolamellar Hepatocellular Carcinoma | FHC | POETIC |
| P01-016 | M | 19 | 1 | 1 | 0 | Osteosarcoma | OS | POETIC |
| P01-017 | M | 5 | 1 | 0 | 1 | Neuroblastoma | NBL | POETIC |
| P01-018 | M | 34 | 1 | 0 | 1 | Neuroblastoma | NBL | POETIC |
| P01-019 | F | 16 | 2 | 1 | 0 | Malignant Rhabdoid Tumor | MRT | POETIC |
| P01-020 | M | 2 | 2 | 1 | 2 | Hepatoblastoma | HB | POETIC |
| P01-021 | M | 3 | 1 | 1 | 1 | Hepatoblastoma | HB | POETIC |
| P01-022 | F | 8 | 5 | 1 | 1 | Embryonal rhabdomyosarcoma | ERMS | POETIC |
| P01-023 | M | 3 | 1 | 0 | 1 | Neuroblastoma | NBL | POETIC |
| P01-024 | M | 1.5 | 1 | 1 | 1 | Wilm's Tumor | WT | POETIC |
| P01-025 | M | 16 | 1 | 0 | 1 | Fibrolamellar Hepatocellular Carcinoma | FHC | POETIC |
| P01-026 | F | 2 | 1 | 0 | 1 | Neuroblastoma | NBL | POETIC |
| P01-027 | F | 19 | 1 | 0 | 0 | Hodgkin's Lymphoma | Hodgkins | POETIC |
| P01-028 | F | 14 | 2 | 1 | 1 | Immature teratoma | Teratoma | POETIC |
| P01-029 | M | 14 | 1 | 1 | 1 | Osteosarcoma | OS | POETIC |
| P01-030 | F | 19 | 1 | 1 | 0 | Osteosarcoma | OS | POETIC |
| P01-033 | M | 7 | 1 | 0 | 0 | Neuroblastoma | NBL | POETIC |
| P01-035 | M | 14 | 1 | 0 | 0 | Desmoplastic Small Round Cell Tumor | DSRCT | POETIC |
| P01-036 | M | 12 | 1 | 0 | 1 | Neuroblastoma | NBL | POETIC |
| P01-037 | F | 4 | 1 | 1 | 1 | Neuroblastoma | NBL | POETIC |
| P01-040 | F | 10 | 1 | 0 | 0 | Neuroblastoma | NBL | POETIC |
| P01-0813 | | | | | 1 | Healthy | P01-0813-T-1 | Conversant |
| P01-2013 | | | | | 1 | Healthy | P01-2013-T-1 | Conversant |
| P01-2714 | | | | | 1 | Healthy | P01-2714-T-1 | Conversant |
| TOTAL: | | | 31 | 13 | 20 | | | |

We also obtained a separate cohort of 44 pediatric cancer tissue samples for validation from the Center for Pathology Research Services at Children's Hospital Los Angeles (CHLA). These samples included 6 cancer types (NBL N=10, OS N=10, WT N=10, DSRCT N=5, Teratoma N=5, ERMS N=4) and were collected under written informed consent and preserved in OCT compound (Table 14). In addition, we included 68 MRT, 221 NBL, 86 OS, 131 WT, and 12 adjacent normal tissue samples from TARGET (total=518), 90 normal tissue samples from ENCODE, and 6426 adult cancer samples from TCGA from 14 tumor types. All three of these cohorts were analyzed on the HM450 array (Illumina) (Table 8, 9, 12).

Sample extraction and library preparation for whole genome bisulfite sequencing. Genomic (g)DNA and total RNA were extracted from flash-frozen normal or tumor tissue using an AllPrep DNA/RNA Mini kit (Qiagen) according to manufacturer's recommendations. Briefly, tissues were homogenized using a Bullet Blender homogenizer (Next Advance) for 5 minutes at full speed with a mixture of 0.9-2.0 mm RNase-free stainless-steel beads. Homogenates were passed through the QIAshredder (Qiagen) to remove any remaining particulate matter. Plasma was isolated from whole blood by spinning it at 300 g for 20 minutes. Cell-free (cf)DNA was extracted from the plasma using the QIAamp DNA Blood Maxi kit (Qiagen) according to the manufacturer's recommendations.

Quantity and purity of the isolated gDNA was determined by Qubit dsDNA High Sensitivity fluorometric assay (Invitrogen), cfDNA was quantitated using the TapeStation High Sensitivity D1000 assay according to the manufacturer's protocol. Extracted gDNA and cfDNA were used for whole genome bisulfite sequencing analysis (WGBS) as described, for example, in Legendre et al., Clin Epigenetics 7, 100 (2015). Directional, bisulfite-converted libraries for paired-end sequencing were prepared using the Ovation Ultralow Methyl-Seq Library System (NuGen), using the manufacturer's suggested protocol. Bisulfite conversion was performed using the EpiTect Fast DNA Bisulfite Kit (Qiagen). Post-library QC was performed on the 4200 Tapestation using High Sensitivity D1000 ScreenTapes (Agilent). Paired-end sequencing was performed on the Illumina NovaSeq 6000 platform using the S2 or S4 flow cell for a total read length of 2×150 bp. Paired-end sequencing on bisulfite treated gDNA and cfDNA was performed. Tissue samples were sequenced by Macrogen on HiSeq X (Illumina). Read pairs were processed through our alignment and methylation calling pipeline which uses Brabham Bioinformatics' Bismark alignment software (Krueger et al., Bioinformatics 27, 1571-1572 (2011)) for read mapping and methylation evaluation. Sequencing of cfDNA was done at USC on Illumina's NovaSeq 6000 using S2 chips. All reads were mapped to hg19.

DMR calling. Differentially methylated regions (DMRs) were evaluated using the DMR caller Metilene (Juhling et al., Genome Res 26, 256-262 (2016)). Tumor samples were compared with their patient-matched adjacent normal sample where possible. For tumor samples without a matched normal, a pool of normal samples from other patients with the same diagnosis was used. For unique cancer types with no matched normal sample, a pool of all normal samples was used. DMRs were filtered to include those with a Mann-Whitney p-value<0.05 and an |Δβ|>0.15. Regions from X and Y chromosome were removed. DMRs were annotated with the name of the nearest gene. Overlapping DMRs were defined as regions in which DMRs for two or more samples shared at least 3 CpGs, with the same directionality (hyper-vs. hypo-methylated).

Partially methylated domain/hypomethylated domain analysis. Partially methylated domains (PMDs) are hypomethylated domains with an intermediate level of methylation spanning several kilobases to a few megabases. PMDs were called using MethPipe (Song et al. PLoS One 8, e81148 (2013)), based on methylation and coverage information from Bismark. We modified methPipe's code to generate additional metrics, namely mean methylation across each PMD and the standard deviation of beta values within each PMD call. Analysis and plotting of PMD regions were performed in R.

CpG minimally differentially methylated regions. We identified a consensus DMR set with substantial enrichment of hypomethylated and hypermethylated DMRs across all samples. Each CpG location was scored by the number of samples with a DMR overlapping this locus (separately for hyper- and hyper-methylated DMRs). These CpGs were filtered to include those with a count of 22 or more samples (70% of samples) and filtered CpGs within 500 bases of each other were combined to form separate regions, termed minimally differentially methylated regions (mDMRs).

Copy number analysis. Copy number variants (CNVs) were called on the WGBS data using the R package QDNAseq (Scheinin et al., Genome Res 24, 2022-2032 (2014)), which uses read counts within fixed sized bins; we selected a 30 kb bin size for evaluation of genome wide copy number variants, and a 1 kb, 5 kb, or 10 kb bin size for focal amplifications such as MYCN gain in neuroblastoma.

Ingenuity pathway analysis. In order to investigate biological pathways associated with DMRs we used Ingenuity Pathway Analysis (IPA, Qiagen). DMRs were annotated with the nearest gene and distance to the nearest gene. The resulting gene list was used as IPA input. We ran pathway enrichment analysis using default IPA settings.

Machine learning classifiers. A mean beta value matrix was used to construct a random forest model using ranger. We used repeated (n=10) 3-fold cross validation to estimate the performance of all models. For the classification of TCGA solid tumors, a single model constructed from beta value of all pediatric solid tissues was used.

Hybridization probe capture. Hybridization probe capture, assay design, sequencing, and bioinformatic analysis were performed as previously described (Buckley et al., NAR Genom Bioinform 4, lqac099 (2022); Buckley et al. Clin Cancer Res, 2023 Dec. 15:29(24):5196-5206).

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A method for determining whether a subject has a pediatric cancer, an adult cancer, or Minimal Residual Disease (MRD) comprising the steps of
a) training a machine learning model to detect the pediatric cancer, the adult cancer, or the MRD, wherein the machine learning model is trained using a first set of target regions from a plurality of cancer samples and corresponding target regions from non-cancerous samples, wherein the plurality of cancer samples comprise at least two different cancer types, wherein the machine learning model is configured to identify the pediatric cancer, the adult cancer, or the MRD based on a comparison of a methylation pattern of the first set of target regions of the plurality of cancer samples compared to a methylation pattern of the corresponding target regions of the non-cancerous samples;
b) determining a methylation pattern of a second set of target regions of a deoxyribonucleic acid (DNA) sample obtained from the subject;
c) applying the trained machine learning model to the methylation pattern of the second set of target regions of the DNA obtained from the subject; and
d) determining that the subject has or does not have the pediatric cancer, the adult cancer, or the MRD based on an output of the machine learning model.

2. The method of claim 1 wherein the methylation pattern of the first set of target regions, the second set of target regions, and the corresponding target regions is determined using DNA methylation analysis, wherein the DNA methylation analysis comprises one or more of whole genome bisulfite sequencing (WGBS), Reduced Representation Bisulfite sequencing (RRBS), Targeted bisulfite sequencing, Hybridization Probe capture, Methylation bead arrays, and Enzymatic methyl-sequence conversion.

3. The method of claim 1 wherein the methylation pattern of the first set of target regions, the second set of target regions, and the corresponding target regions is determined using hybridization probe capture after whole genome bisulfite sequencing.

4. The method of claim 3 wherein the hybridization probe capture comprises one or more probes that hybridize to the one or more target genomic regions, wherein each of the one or more probes comprises ribonucleic acid or deoxyribonucleic acid, and optionally, wherein each of the one or more probes comprises an affinity tag selected from the group consisting of biotin and streptavidin.

5. The method of claim 4 wherein the one or more probes are affixed to a solid support.

6. The method of claim 1 wherein the DNA sample comprises cell free DNA (cfDNA) or genomic DNA isolated from tissue or cells.

7. The method of claim 6 wherein the cf DNA is extracted from one or more of whole blood, plasma, serum, urine, ascites, cerebral spinal fluid, and aspirates.

8. The method of claim 1 wherein the pediatric cancer or the adult cancer is osteosarcoma, medulloblastomas, ependymomas, optical nerve gliomas, brain stem glioma, oligodendrogliomas, gangliogliomas, Pineal Region Tumors, hepatoblastoma, Fibrolamellar Hepatocellular Carcinoma, Hodgkin's lymphoma, Non-Hodgkin's lymphoma, acute lymphoblastic leukemia, acute myeloid leukemia, juvenile myelomonocytic leukemia, acute promyelocytic leukemia, chronic lymphoblastic leukemia, chronic myeloid leukemia, diffuse intrinsic pontine glioma, neuroblastoma, retinoblastoma, rhaboid tumors, Ewing's sarcoma, rhabdomyosarcoma, embryonal rhabdomyosarcoma, fibrosarcoma, mesenchymoma, synovial sarcoma, a teratoma, liposarcoma, spinal cord tumors, ovarian cancer, or Wilm's tumors.

9. The method of claim 8 wherein the pediatric cancer or the adult cancer is a stage I, stage II, stage III, or stage IV cancer.

10. The method of claim 1 wherein the first set of target regions, the second set of target regions, and the corresponding target regions comprises about 30% to about 50% of the target regions of Table 1.

11. The method of claim 1 wherein the first set of target regions, the second set of target regions, and the corresponding target regions comprise about 50% to about 70% of the target regions of Table 1.

12. The method of claim 1 wherein the first set of target regions, the second set of target regions, and the corresponding target regions comprise about 70% to about 90% of the target regions of Table 1.

13. The method of claim 1 wherein the first set of target regions, the second set of target regions, and the corresponding target regions comprise about 90% to about 95% of the target regions of Table 1.

14. The method of claim 1 wherein the first set of target regions, the second set of target regions, and the corresponding target regions target regions comprise greater than about 95% of the target regions of Table 1.

15. The method of claim 1 further comprising treating the pediatric cancer or the adult cancer, or the MRD in the subject, wherein the treatment comprises one or more of radiation therapy, surgery to remove the cancer, and administering a therapeutic agent to the subject.

16. The method of claim 1 wherein the machine learning model comprises one or more of Random Forest, a support vector machine (SVM), a neural network, Generalized Linear Model (GLM), Gradient Boosted Model (GBM), Extreme Gradient Boosting (XGB), and a deep learning algorithm.

17. The method of claim 1 wherein the first set of target regions comprise the target regions of Table 1.

18. The method of claim 17 further comprising comparing a methylation level of sequence reads of the first set of target regions of the plurality of cancerous samples to a methylation level of the corresponding target regions of the non-cancerous samples to identify differentially methylated regions (DMRs) between the plurality of cancerous sample and the non-cancerous sample.

19. The method of claim 18 further comprising identifying the DMRs common to the plurality of cancerous samples to define a set of minimally differentially methylated regions (mDMRs), wherein the mDMRs are used to train the machine learning model.

20. A kit comprising a plurality of polynucleotide probes that are complementary to the plurality of target regions listed in Table 1.
